(19) United States
(12) Patent Application Publication
(10) Pub. No.: US 2022/0111034 A1
(43) Pub. Date: Apr. 14, 2022

(54) VIRUS COMPOSITIONS

(71) Applicant: Université Catholique de Louvain, Louvain-Ia-Neuve (BE)

(72) Inventors: David Alsteens, Corroy-le-grand (BE); Melanie Köhler, Louvain-la-Neuve (BE)

(21) Appl. No.: 17/423,765

(22) PCT Filed: Jan. 17, 2020
(86) PCT No.: PCT/EP2020/051114

§ 371 Date: Jul. 16, 2021

(30) Foreign Application Priority Data
Jan. 18, 2019 (EP) 19152640.9

(51) Int. Cl. A61K 39/15; A61K 35/765; A61K 39/39

(52) U.S. Cl. CPC A61K 39/15; A61K 2039/5254; A61K 39/39; A61K 35/765

(57) ABSTRACT

Aspects of the invention concern a composition or a kit-of-parts comprising i) a virus which is a member of the Reoviridae family and ii) sialic acid and/or a molecule comprising at least one sialic acid moiety, as well therapeutic applications thereof.

### FIELD

Aspects of the invention are broadly in the medical field and more specifically concern compositions or kits-of-parts comprising a virus which is a member of the Reoviridae family. The disclosed compositions or kits-of-parts are particularly useful in therapy, such as for example in methods of treating neoplastic diseases or in immunisation methods. The invention also encompasses methods for making or using the disclosed compositions or kits-of-parts.

### BACKGROUND

Viruses are strict intracellular parasites and because of their simplicity, they depend on a host organism in virtually all stages of the replication cycle. During evolution and adaptation to their hosts, they acquired the relevant molecular ‘keys’ or ‘entrance tickets’ to be able to exploit and control cellular functions. Virus entry is largely defined by the interactions between virus particles and their receptors at host cell surfaces. These interactions determine the mechanism of virus attachment, uptake, intracellular trafficking, and, ultimately penetration to the cytosol.

The viruses of family Reoviridae are ribonucleic acid (RNA) viruses, lack an outer lipid envelope, appear spheroidal, measure about 60-100 nanometres across, have two capsids (or concentric shells, commonly called outer capsid and inner core), and contain a core of segmented, double-stranded RNA. Reoviridae viruses are currently grouped into two sub-families, Sedoreovirinae and Spinareovirinae, including numerous genera, of which Orthoreovirus, Orbivirus, and Rotavirus are among the best known. Reoviridae viruses have a wide host range, including vertebrates, invertebrates, plants, protists, and fungi. For example, certain Orthoreovirus, Orbivirus, Coltivirus, and Rotavirus species infect humans, certain Orthoreovirus species infect birds, Phytoreovirus and Fyivirus species infect plants and insects, Cypovirus species infect insects, and Aquareoviruses infect fish.

Infections with mammalian orthoreoviruses (reoviruses) may be frequent though typically mild or subclinical, but a recent study in humans supports a role for reovirus infection in triggering the development of celiac disease by breaking oral tolerance to gluten (Bouziat, R. et al. Reovirus infection triggers inflammatory responses to dietary antigens and development of celiac disease. Science 2017, vol. 356, 44-50). Further, rotavirus is a major human pathogen causing infective infantile diarrhoea, and the use of rotavirus vaccines such as Rotarix (GlaxoSmithKline) or RotaTeq® (Merck Vaccines) to protect infants is commonplace.

Reovirus infections are pervasive in commercial poultry flocks. Most strains are non-pathogenic and appear to survive harmlessly in the intestine. However, others have been associated with several disease conditions. The most frequent reovirus-associated disease in poultry is viral arthritis, which manifests in swelling of the tendons of the shank and above the hock. Affected birds walk with a stiff gait or prefer not to move. Vaccines against reovirus infection in chickens are commercially available, for example Nobilis® REO 1133 from MSD Animal Health.

In addition, reoviruses are promising oncolytic agents, as reovirus selectively targets transformed cells with activated Ras signalling pathways, and at least about 30% of human tumors exhibit aberrant Ras signalling. By targeting Ras-activated cells, reovirus can directly lyse cancer cells, disrupt tumor immunosuppressive mechanisms, re-establish multicellular immune surveillance and generate robust anti-tumor responses (Duncan et al. Differential sensitivity of normal and transformed human cells to reovirus infection. J. Virol. 1978, vol. 28, 444-449; Coffey et al. Reovirus therapy of tumors with activated Ras pathway. Science 1998, vol. 282, 1332-1334). Reovirus has shown efficacy in clinical trials for refractory human cancers (Mahalingam et al. A phase II study of pelareorep (REOLYSIN®)) in combination with Gemcitabine for patients with advanced pancreatic adenocarcinoma. Cancers (Basel) 2018, vol. 10, E160; Samson et al. Oncolytic reovirus as a combined antiviral and anti-tumour agent for the treatment of liver cancer. Gut 2018, vol. 67, 562-573; Samson et al. Intravenous delivery of oncolytic reovirus to brain tumor patients immunologically primes for subsequent checkpoint blockade. Science translational medicine 2018, vol. 10, eaam7577).

Among their viral outer capsid proteins, sigma-1 (σ1) protein emerges as a determinant to reovirus entry (Stencel-Baerenwald et al. The sweet spot: defining virus-sialic acid interactions. Nat. Rev. Microbiol. 2014, vol. 12, 739-749). σ1 protein is a fibrous trimer that consists of two domains, an elongated tail domain linked to the viral particle and a globular head that is projected away from the viral particle surface. Both parts contain receptor-binding domains. The tail domain is able to engage cell surface carbohydrate containing α-linked sialic acid (α-SA), whereas the head domain binds junctional adhesion molecule A (JAM-A) (Danthi et al. Reovirus receptors, cell entry, and proapoptotic signaling. Adv. Exp. Med. Biol. 2013, vol. 790, 42-71). Recent studies showed that single point mutation in the al tail region implicated in α-SA binding is responsible for the serotype-dependent differences in reovirus tropism, more particularly influences the neurovirulence of serotype 3 reovirus (Frierson et al. Utilization of sialylated glycans as coreceptors enhances the neurovirulence of serotype 3 reovirus. J. Virol. 2012, JVI. 01822-01812), while JAM-A receptor serves as a receptor for all three reovirus serotypes (Stencel-Baerenwald et al. 2014, supra; Barton et al. Utilization of sialic acid as a coreceptor enhances reovirus attachment by multistep adhesion strengthening. J. Biol. Chem. 2001, vol. 276, 2200-2211 (‘Barton et al. 2001a’); Barton et al. Junction adhesion molecule is a receptor for reovirus. Cell 2001, vol. 104, 441-451 (Barton et al. 2001b′)).

### SUMMARY

The present invention is at least in part based on the inventors' meticulous effort to unravel the molecular mechanisms for reovirus binding to cell surface molecules, leading to the unexpected discovery that sialic acid (SA) binding to the reovirus sigma 1 (σ1) protein acts as a trigger of σ1-binding potential to the JAM-A surface receptor, which is a key step in viral entry. Without wishing to be bound by any theory, the inventors more particularly discovered that SA interaction with the reovirus σ1 protein actively promotes a conformational change in the σ1 protein towards a more elongated or extended conformation, and that this conformational change in the σ1 protein results in an increased ability of the virus to bind the cognate cell surface receptors, in particular JAM-A, significantly increasing the number of bonds established between the virus and the cell surface. This increased binding can in turn confine the virus on the cell surface and thus favour its entry into the cytosol.

These observations for the first time identify sialic acid or molecules comprising sialic acid moiety or moieties as potent enhancers of reovirus binding to cells and infectivity, and provide a dependable avenue for increasing the effectiveness of methods relying on reovirus cell entry, such as reovirus-based therapies, for example therapies which employ the oncolytic properties of reovirus, or therapies which involve vaccination against reovirus.

Accordingly, an aspect provides a composition comprising i) a virus which is a member of the Reoviridae family and ii) sialic acid and/or a molecule comprising at least one sialic acid moiety.

Another aspect provides a kit-of-parts comprising i) a virus which is a member of the Reoviridae family and ii) sialic acid and/or a molecule comprising at least one sialic acid moiety.

A further aspect provides the composition for use in therapy. A related aspect provides use of the composition in therapy.

A further aspect provides the kit-of-parts for use in therapy. A related aspect provides use of the kit-of-parts in therapy.

A further aspect provides a method for treating a subject in need thereof, the method comprising administering to the subject a prophylactically or therapeutically effective amount of i) a virus which is a member of the Reoviridae family and ii) sialic acid and/or a molecule comprising at least one sialic acid moiety.

A further aspect provides an in vitro method for propagating a virus which is a member of the Reoviridae family, the method comprising: i) infecting a host cell susceptible to infection by said virus, wherein the host cell has been genetically engineered to overexpress JAM-A, with said virus, either in the presence of sialic acid and/or a molecule comprising at least one sialic acid moiety, or wherein said virus has been previously treated with sialic acid and/or a molecule comprising at least one sialic acid moiety; ii) allowing the virus to propagate in said host cell; and optionally iii) isolating the propagated virus produced by the host cell.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 illustrates principle of FD-based AFM to probe reovirus binding to living cells. (a) The AFM is placed on an optical microscope. CHO or Lec2 cells are maintained in a specially designed cell culture chamber, which allows control of temperature and the gas atmosphere and prevents the medium from evaporation. (b) The AFM cantilever, bearing the tip functionalized with the virus of interest, is oscillated with frequency in the kHz range with a sinusoidal driving motion inducing approach and retraction movements towards the sample. (c, d) The recorded tip-sample interactions are displayed as force vs. time (c) or force vs. distance, (d) which allows tracking of forces established towards the biological sample. (e) Mechanical properties (including adhesion) can be extracted from individual force curves and directly correlated with their position on the sample (e.g., height image and corresponding adhesion map).

FIG. 2 illustrates characterization of cell surface receptor expression by cell lines used in the Examples. (a) Flow cytometry profiles (left) and corresponding quantification of median fluorescence intensities (right) of JAM-A expression. JAM-A was detected using a monoclonal antibody and indirect immunofluorescence. (b) CHO and Lec2 cell lines were analyzed for expression of cell-surface sialic acid by incubation with fluorescent lectin (wheat germ agglutinin, WGA). Graphs show cytometry profiles of WGA bound to indicated cell lines (left) and quantification of median fluorescence intensity of bound lectin (right).

FIG. 3 illustrates probing T3 reovirus binding to sialylated glycans on model surfaces and living cells. (a) Binding of single virions is probed on an SA-coated surface in the presence or absence of α-SA glycan derivatives: N-acetylneuraminic acid (Neu5Ac), sialyl-lacto-N-tetraose a (LSTa), and a derivative without α-SA (lacto-N-neotetraose [LNnT]). (b) Box plot of the specific binding frequency measured by AFM between virions and α-SA determined without and following injection of 1 mM glycans. (c) Dynamic force spectroscopy (DFS) plot showing the distribution of rupture force measured between T3SA+ and the SA-coated surface (grey dots) with average rupture forces determined for eight distinct loading rate (LR) ranges. Data corresponding to single interactions are fitted with the Bell-Evans (BE) model describing a ligand-receptor bond as a simple two-state model (I, black curve). Dashed lines represent the predicted binding forces for two (II) and three (III) simultaneous uncorrelated interactions (Williams-Evans model [WEM]). Insert: BE model describing a ligand-receptor bond as a simple two-state model. The bound state is separated from the unbound state by an energy barrier located at distance xᵤ. kᵤ and k_{off} represent the transition rate and transition at thermal equilibrium, respectively. (d) Combined optical microscopy and FD-based AFM of T3SA+ binding to cells expressing (CHO) or lacking (Lec2) α-SA on the cell surface. (e) Overlay of DIC, GFP, and mCherry signals of a confluent layer of cocultured fluorescent CHO cells (actin-mCherry and H2B-eGFP) and Lec2 cells. (f, g) FD-based AFM topography image (f) and corresponding adhesion map (g) of adjacent cells probed indicated in the dashed square in (e). The adhesion map shows interactions mainly on CHO cells (α-SA+ cells) (white pixels). (h) DFS plot of data from α-SA model surfaces (grey circles, from b) and living cells (red dots). Histogram of the force distribution observed on cells fitted with a multi-peak Gaussian distribution (n=700) is shown at the side. (i) Box plot of BF observed for T3SA+ (grey) and T3SA− (white) virions as well as T3SA+ virions following injection of 1 mM Neu5Ac (red). Error bars indicate standard deviation (s.d.). For all experiments, data are representative of at least five independent experiments. (j) Influence of SA on virus binding determined by flow cytometry. Cells were incubated with either PBS(Mock) or Alexa Flour 488-labeled T3SA+ or T3SA− virions (10⁵ particles per cell), and the median fluorescence intensity (MFI) of cell-bound virus was determined by flow cytometry. Error bars indicate s.d. Experiments were repeated twice, each with duplicate samples. ****; P<0.0001; determined by two-way ANOVA corrected for multiple comparisons using Tukey's test in GraphPad Prism or Origin.

FIG. 4 illustrates probing reovirus binding to JAM-A on model surfaces and living cells. (a) Binding of single virions (T3SA+ or T3SA−) to JAM-A probed on a model surface. (b) DFS plot showing the force required to separate T3SA+ (upper panel) or T3SA− (lower panel) virions from JAM-A and fitted with the BE model. (c) Box plot of the binding frequency of reovirus to a JAM-A model surface. T3SA+ interactions are shown in grey, T3SA− in white, and hatched boxes represent injection of 10 μg/mL JAM-A antibody (AB). (d) Combined optical and FD-based AFM of T3SA+ interaction with JAM-A on living Lec2 cells. (e) Overlay of DIC, GFP, and mCherry signals of a confluent layer of cocultured fluorescent Lec2 cells (actin-mCherry and H2B-eGFP) and unlabeled Lec2-JAM-A cells. (f, g) FD-based AFM topography image (f) and corresponding adhesion map (g) of adjacent cells indicated in the dashed square in (e). The adhesion map shows interactions mainly between T3SA+ particles and Lec2-JAM-A cells (white pixels). (h) DFS plot of T3SA+ interactions with JAM-A on model surfaces (grey circles, taken from b—upper panel) and living cells (red dots). Histogram of the force distribution observed on cells fitted with a multi-peak Gaussian distribution (n=600) is shown on the side. (i) Box plot of BF observed for T3SA+ (grey) and T3SA− (white) virions, with (hatched lines) and without injection of JAM-A AB (10 μg/ml). Error bars indicate s.d. For all experiments, data are representative of at least five independent experiments. (j) Influence of JAM-A on virus binding determined using flow cytometry. Cells were incubated with either no virions (Mock) or Alexa Flour 488-labeled T3SA+ or T3SA− virions (10⁵ particles per cell), and the median fluorescence intensity (MFI) of cell-bound virus was determined by flow cytometry. Error bars indicate s.d. Experiments were repeated twice, each with duplicate samples. *, P<0.05; ****; P<0.0001; determined by two-way ANOVA corrected for multiple comparisons using Tukey's test in GraphPad Prism or Origin.

FIG. 5 illustrates influence of sialylated glycans on reovirus binding to JAM-A. (a) Binding of T3SA+ or T3SA− virions or T3SA+ISVPs to JAM-A was monitored following injection of 1 mM α-SA glycans (Neu5Ac [b, red] and LSTa [c, yellow]) or non-sialylated glycan (LNnT [d, green]). (b-d) DFS plots of interaction forces measured between T3SA+ and JAM-A after adding 1 mM glycan (Neu5Ac in b, LSTa in c, and LNnT in d). Grey dots represent the measured binding forces before injection. (e) DFS plot of the interaction forces between JAM-A and T3SA+ISVPs, which display a more extended conformation of the 61 protein. Multivalent interactions are observed for T3SA+ISVPs (blue) in comparison to T3SA+ virions (grey) without injection of free SA. (f) Relative frequency of single and multiple bonds before and after adding free glycans was determined from the areas under the respective peaks within force distribution histograms. (g) Number of bonds established between JAM-A and T3SA+ (left panel) or T3SA− (middle panel) virions or T3SA+ISVPs (right panel), before (grey) and after injection of sialylated (Neu5Ac—red, LSTa—yellow) or non-sialylated (LNnT—green) glycans. The grey (‘before’) curve follows substantially the same profile as the LNnT curve. Error bars indicate s.d. of the mean value. For all experiments, data are representative of at least n=3 independent experiments.

FIG. 6 illustrates testing the effect of free SA compounds on T3SA− binding to JAMA. (a-c) DFS plots of the interaction between T3SA− and JAM-A after adding 1 mM Neu5Ac (a, red), 1 mM LSTa (b, yellow), or 1 mM LNnT (lacking SA group) (c, green) probed on model surfaces. Overlaid grey circles represent the binding events before injection of the compounds. Single JAM-A-T3SA interactions are observed in all four experiments and fitted with the Bell-Evans model (black line). In contrast to the results shown in FIG. 5, injection of Neu5Ac or LSTa does not induce any change in JAM-A-T3SAbinding or establishment of multivalent interactions, evidencing that the sialic acid binding site in T3SA+ is responsible for this observation. (d) Box plot of BF observed for JAM-A-T3SA+ (left panel), JAMA-T3SA− (middle panel), and JAMA-T3SA+ISVP (right panel) interactions, without adding SA compounds (grey for T3SA+, white for T3SA−, and blue for T3SA+ISVP) and after adding Neu5Ac (red), LSTa (yellow), or LNnT (green), as well as after injection of 10 μg/ml JAM-A AB as a receptor-blocking reagent (dashed lines in the respective boxes). The horizontal line within the box indicates the median, boundaries of the box indicate the 25^{th} and 75^{th} percentile, and the whiskers indicate the highest and lowest values of the results. The square in the box indicates the mean. The observed reduction in binding frequency in the presence of JAM-A AB verifies the specificity of observed interactions. For all experiments, data are representative of at least n=3 independent experiments. ****, P<0.0001; determined by two-sample t-test in Origin. Error bars indicate s.d. of the mean value.

FIG. 7 illustrates probing reovirus binding to living cells. (a) Schematic of reovirus particles with outer-capsid proteins labeled before (virion) and after (infectious subvirion particle [ISVP]) protease treatment. The cartoon shows the arrangement of outer-capsid proteins in the double-layered shells of virions and the formation of ISVPs by removal of σ3, cleavage of μ1 to yield δ and ϕ, and rearrangement of σ1 into a more elongated conformation. (b) Full-length model of reovirus σ1 protein (Dietrich et al. Structural and Functional Features of the Reovirus σ1 Tail. J. Virol. 2018, JVI 00336-00318), which functions as the viral attachment protein that binds to cell-surface glycans (in particular to terminal α-linked sialic acid [α-SA] residues) and junctional adhesion molecule-A (JAM-A). Regions of the molecule that interact with α-SA and JAM-A are indicated. (c) Schematic of probing reovirus entry using AFM. The initial attachment of reovirus to cells involves specific binding between the viral σ1 protein and the receptor, JAM-A. Cell-surface glycans function as co-receptors.

FIG. 8 illustrates monitoring the effect of SA addition on reovirus binding to living cells. T3SA+ binding to Lec2-JAM-A cells was assessed before and after adding 1 mM Neu5Ac (a-e), 1 mM LSTa (f-j), or 1 mM LNnT (k-o). (a,f,k) AFM topography image of adjacent Lec2 and Lec2-JAM-A cells with fluorescent image (20×20 μm) inset showing a fluorescently tagged Lec2 cell lacking JAM-A expression. (b,g,l) Corresponding adhesion map recorded before injection of glycan. (c,h,m) Enlarged images of adhesion maps recorded on Lec2-JAM-A cells (dashed square in adhesion map). The upper images (light grey frame) display the lower force range (300 to 400 pN), whereas the lower images (dark grey frame) display the higher force range (400 to 500 pN), with significantly fewer adhesion events. (d,i,n) Adhesion maps recorded following injection of free Neu5Ac (d), LSTa (i), or LNnT (n). The area probed is similar to the area recorded before glycan injection. (e,j,o) Enlarged images of adhesion maps recorded on Lec2-JAM-A cells (dashed square in adhesion map and same areas as in b,g,i show more adhesion events in the high force range for sialylated glycan [Neu5a and LSTa] and no significant change for non-sialylated glycan [LNnT]). The frequency of adhesion events is indicated. (p-s) Histogram of the force distribution extracted on Lec2-JAM-A cells (dashed square in adhesion map) before (p) and after adding Neu5Ac (q), LSTa (r), and LNnT (s). Histograms were fitted with a multi-peak Gaussian distribution. (t) Number of bonds established between JAM-A cell-surface receptors and T3SA+ before (grey) and after injection of sialylated or non-sialylated glycans (colored). Error bars indicate s.d. of the mean value. The statistical analysis is shown in Table 1. For all experiments, data are representative of at least n=15 cells from n=5 independent experiments.

Table 1. Statistical analysis of the number of bonds established between JAM-A cell-surface receptors and T3SA+ virions under different conditions. P values were derived from comparisons of data before and after injection of the sialylated glycan (Neu5Ac, LSTa) or non-sialylated glycan (LNnT). The number of bonds established after injection of sialylated glycans differs significantly in comparison with the data before or after injection of the non-sialylated glycan. ns, P >0.05; **, P<0.01; ***, P<0.001; ****, P<0.0001; determined by two-sample t-test in Origin. For all experiments, data are representative of at least n=15 cells from n=5 independent experiments.

| # bonds | +Neu5Ac | +LSTa | +LNnT |
|---|---|---|---|
| I | ns | ns | ns |
| II | **** | **** | ns |
| III | ** | ** | ns |
| IV | **** | *** | ns |
| V | **** | **** | ns |
| VI | **** | **** | ns |

FIG. 9 illustrates monitoring the effect of SA addition on reovirus binding to living cells. Box plot of the BF observed for T3SA+ virions with (dashed lines) and without injection of JAM-A AB (10 μg/ml) as well as after adding the indicated glycans. Data are representative of at least five independent experiments.

FIG. 10 illustrates triggering multivalent anchorage of reovirus virions alters diffusion potential and binding behavior in bulk. (a, b) Biolayer interferometry data for the binding of reovirus (T3SA−, T3SA+ and T3SA+ISVP) to JAM-A receptor immobilized on NTA-coated biosensors. The effect of addition of 1 mM Neu5Ac in solution was tested for both T3SA− and T3SA+. Sensorgram starts with baseline (BL) measurement following by the immobilization of JAM-A to the NTA biosensor (loading), the addition of the virions (association), and finally by the dissociation phase. (c-g) Real-time confocal fluorescence imaging of reovirus particles (labeled with Alexa488 dye) incubated on cocultured CHO-JAM-A and Lec2-JAM-A cells in the absence (c, d) and presence (e, f) of 1 mM Neu5Ac. (c, e) Overlay images of Alexa488 (virions), mCherry-actin of Lec2-Jam-A, and PMT signals. (d, f) Time-lapse trajectories of T3SA+ particles. White and yellow trajectories represent the movement on Lec2-JAM-A cells and CHO-JAM-A cells, respectively. Magnification of each trajectory is shown on the right side with the corresponding number. (g) Analysis of the mean travelled distance (top panel), mean travel speed (middle panel), and bound viral particles (bottom panel) for T3SA+ binding in the absence (grey) or presence (red) of Neu5Ac as well as for T3SA− binding in the absence (white) or presence (light red) of Neu5Ac following adsorption to the cell mixture. The horizontal line within the box plot (bottom panel) indicates the median, boundaries of the box indicate the 25^{th} and 75^{th} percentile, and the whiskers indicate the highest and lowest values of the results. The square in the box indicates the mean. Data are representative of at least three independent experiments, with a minimum of 15 analyzed trajectories each. ***, P<0.001; ****; P<0.0001; determined by two-sample t-test in Origin. Error bars indicate s.d. of the mean value. (h) Model depicting how binding to α-SA triggers a conformational change in the σ1 protein leading to a more extended conformation, resulting in an increased affinity for JAM-A.

FIG. 11 illustrates sialic acid moiety structures commonly found in vertebrate systems, which may be useful in certain embodiments of the invention.

FIG. 12 illustrates characterization of reovirus particles and validation of tip and surface immobilization. (a, b) AFM height images of reovirus particles deposited on freshly cleaved HOPG substrate at low (a) and high (b) magnification. Insert: 3D reconstruction. (c) Z-stack image of an AFM tip functionalized with reovirus obtained by laser-scanning optical microscopy after staining with primary antibody against reovirus and APC-conjugated secondary antibody (red). The inset image highlights the virion link at the tip apex. Experiments were repeated three times with similar results. (d, e) AFM topography image of a SA (d) or JAM-A (e) coated surface after scanning a 500×500 nm area at high forces (˜18 nN) to remove the attached biomolecules (referred to as “scratching” experiment). Insets: Cross-sections taken along the white dashed line in d and e, showing an accumulation of biological materials on the sides of the square. The biomolecule-free surface of inside the square was ˜1 nm (d) or ˜2 nm (e) lower than the surrounding biomolecule-coated surface, providing an estimate of the thickness of SA (d) or JAM-A (e) deposited layer.

FIG. 13 illustrates characterization of cell surface receptor expression by cell lines used in the study, in particular gating strategy used for flow cytometry analysis created from a representative data set. In the first two gating steps, forward and side scatter were used to select for single cells, which were subsequently gated for live cells using the LIVE/DEAD fixable violet dead cell stain kit (Invitrogen). Median fluorescence intensity (MFI) of live cells in the channel of interest was then determined.

FIG. 14 illustrates control experiments for studying the SA contribution in reovirus binding to living cells. (a-d) Consecutive mapping of T3SA+ virus binding to the cell-mixture show similar results. (a) Cartoon of the experiment highlighting that CHO cells are fluorescently labeled. FD-based AFM height image (b) (25 μm×25 μm fluorescent image of the cells) and corresponding adhesion channels show similar results for two consecutive maps (c, d), indicating that the virus was firmly attached to the tip and did not degrade over time. (e-h) Same areas on the cell were consecutively probed with T3SA+ and T3SA− virions. (e) Cartoon of the experiment. (f) FD-based AFM height image and corresponding adhesion forces, acquired first with T3SA+ virions on the tip (f, g), followed by scanning the same area with T3SA− virions on the tip (h). The significant decrease in adhesion (white pixels) on CHO cells after changing the tip to the non-SA-binding virus supports the specificity of probing cell-surface sialic acid interactions with T3SA+. As another control experiment for SA-specific binding, blocking studies were conducted to test inhibition of T3SA+ interactions (as shown in i-n). (i) Cartoon of the experiment. (j) FD-based AFM height image and corresponding adhesion forces acquired first with T3SA+ virions on the tip (j, k), followed by scanning the same area after injection of 1 mM Neu5Ac (l) that can bind to and block reovirus interaction with cell surface SA. A significant reduction of adhesion events can be seen. All AFM images were acquired using an oscillation frequency of 0.25 kHz and amplitude of 750 nm, under cell culture conditions. Experiments were repeated 5-10 times. For higher visibility, the pixel size in the adhesion image was enlarged two-fold.

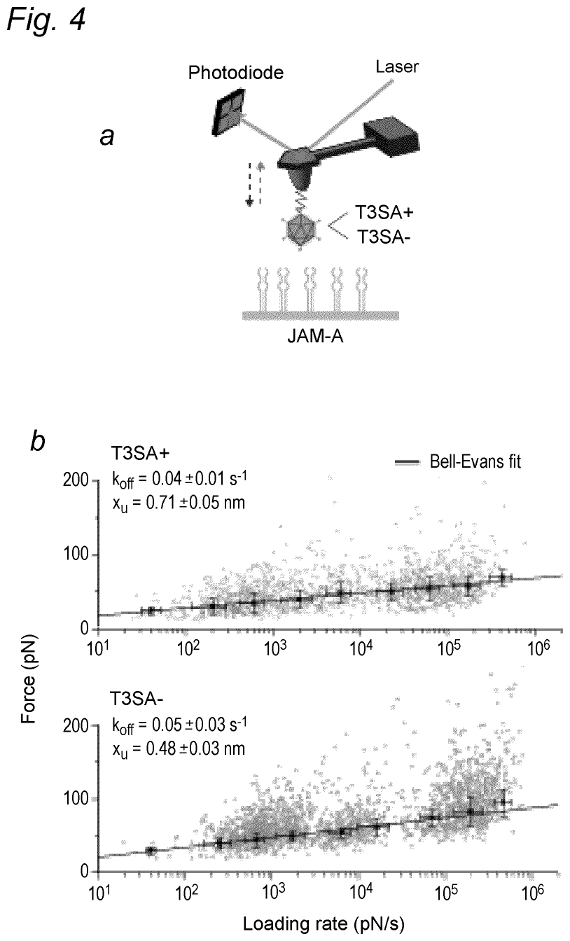

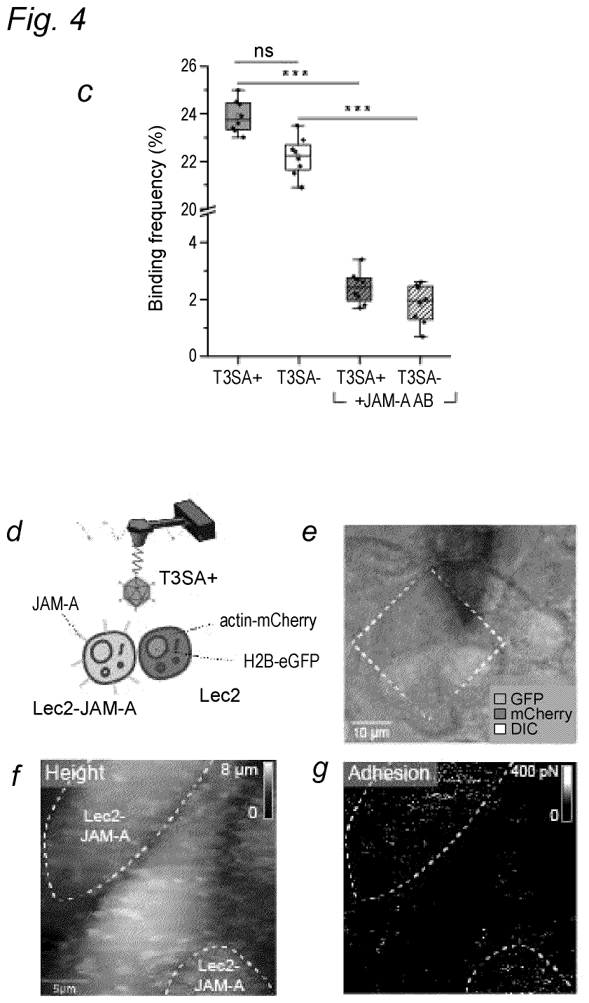

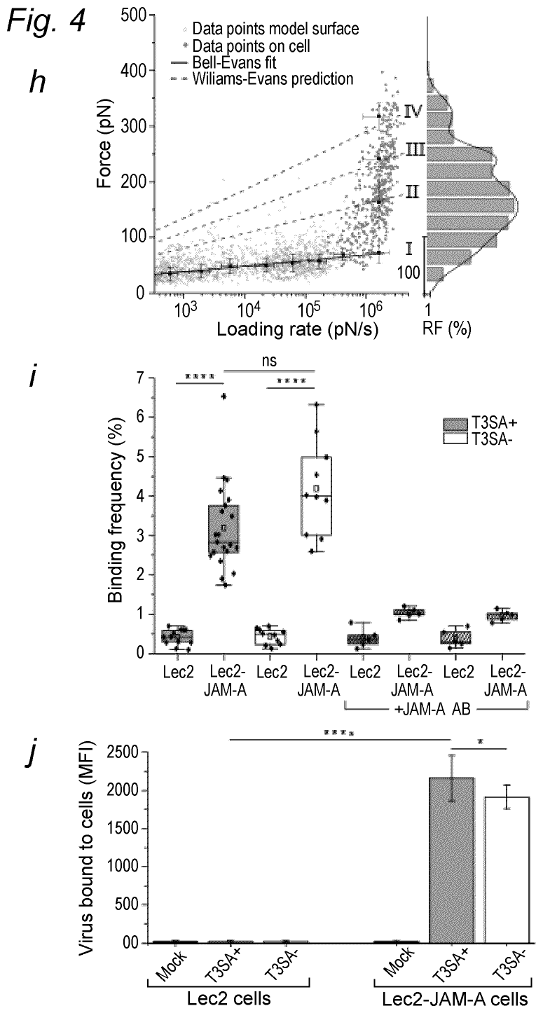

FIG. 15 illustrates control experiments for studying the contribution of JAM-A in reovirus binding to living cells. (a-d) Consecutive mappings of T3SA+ virus binding to Lec2 and Lec2-JAM-A cell-mixture show similar results. (a) Cartoon of the experiment highlighting that Lec2 cells are fluorescently labeled. FD-based AFM height image (b) (25 μm x 25 μm fluorescent image of the cells is shown in inset) and corresponding adhesion channels show similar results for two consecutive maps (c, d), indicating that the virus was firmly attached to the tip and did not degrade over time. (e-h) Same areas on cells were probed with first a T3SA+ virion and then with a T3SA− virion. (e) Cartoon of the experiment. (f) FD-based AFM height image and corresponding adhesion channels, acquired first with T3SA+ virions on the tip (f, g), followed by scanning the same area with T3SA− virions on the tip (h). Both adhesion images show similar results, indicating that JAM-A is engaged in reovirus binding independent of the presence of SA binding site on the virus. (i-j) DFS analysis of T3SA interactions with JAM-A extracted from adhesion areas on Lec2-JAM-A cells. (i) Cartoon of the experiment. (j) DFS plot of T3SA− interactions with JAM-A on model surfaces (grey circles, taken from FIG. 4b—lower panel) and living cells (red dots). Histogram of the force distribution observed on cells fitted with a multi-peak Gaussian distribution (n=620 data points) is shown on the side. Error bars indicate s.d. of the mean value. (k-n) As another control experiment for JAM-A-specific binding, the effect of cell-surface-receptor-blocking reagents on T3SA+ interactions was tested. (k) Cartoon of the experiment. (l-n) FD-based AFM height images and corresponding adhesion images, acquired first with T3SA+ virions on the tip without blocking reagents (l, m), followed by scanning the same area after injection of 10 μg/ml JAM-A Ab (n) to block cell-surface JAM-A molecules. A significant reduction of adhesion events was observed. All AFM images were acquired using an oscillation frequency of 0.25 kHz and amplitude of 750 nm under cell culture conditions. Experiments were repeated 5-10 times. For higher visibility, the pixel size in the adhesion image was enlarged two-fold.

FIG. 16 illustrates testing the effect of free SA compounds on T3SA− binding to JAM-A, in particular DFS plot of the interaction between T3SA+ISVP and JAM-A after adding 1 mM Neu5Ac (red) probed on model surfaces. Neu5Ac does not induce any change in the multivalent binding behavior from that observed in the absence of free glycan.

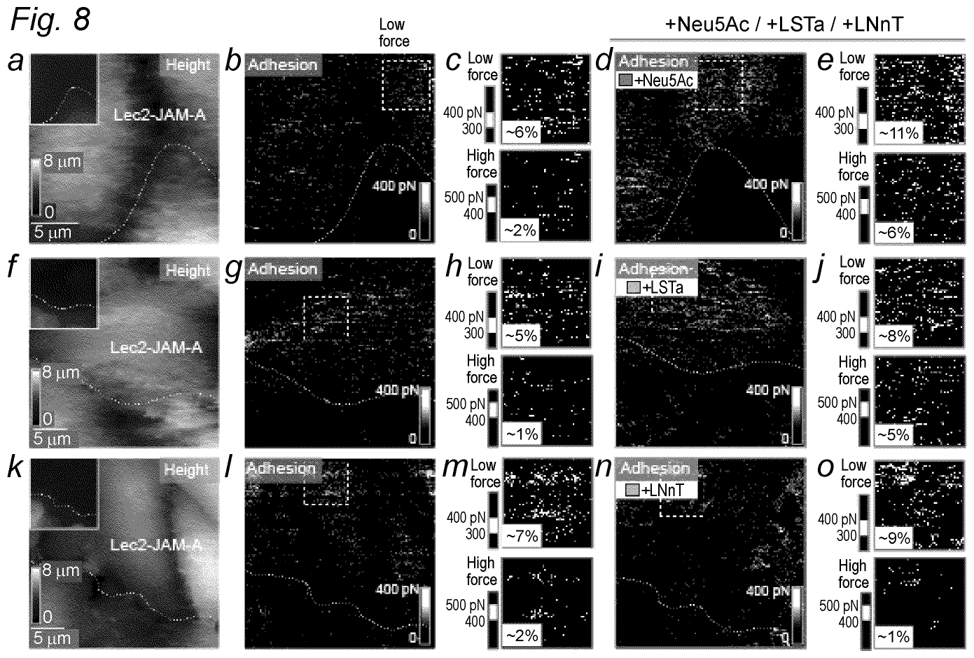

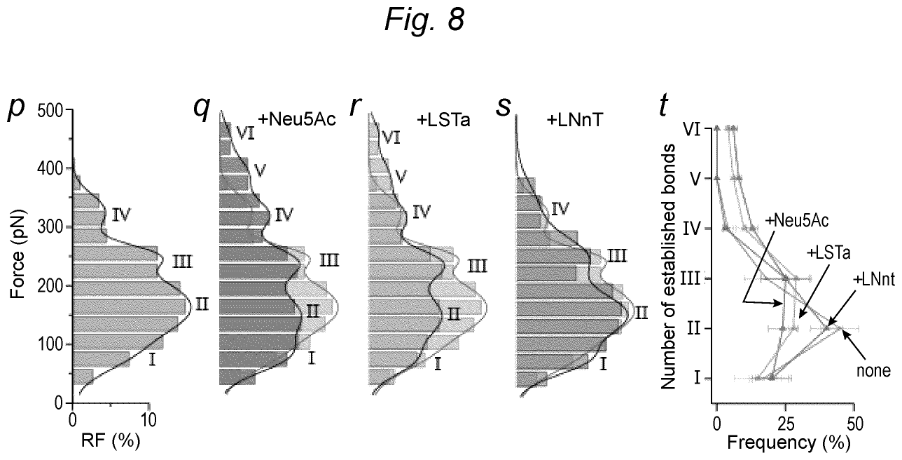

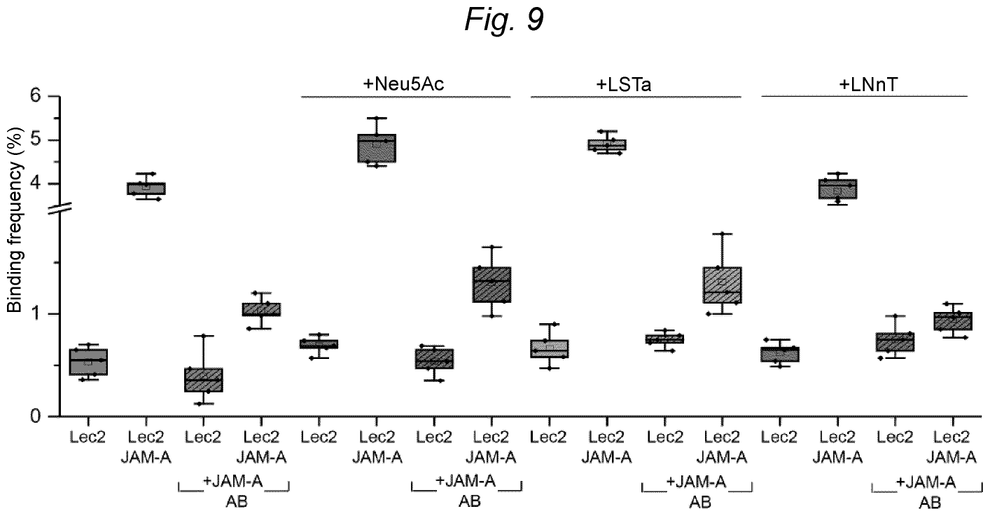

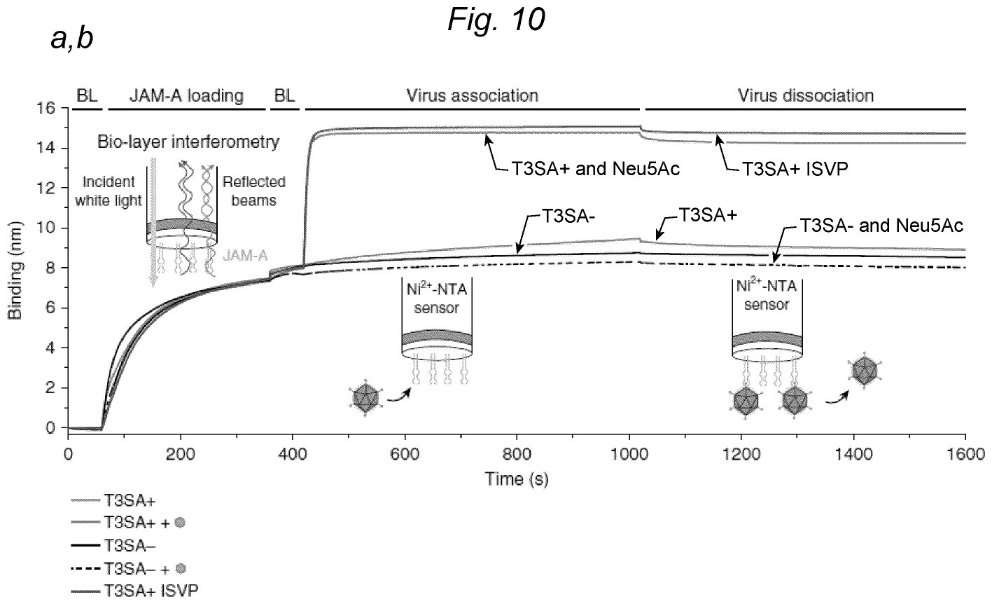

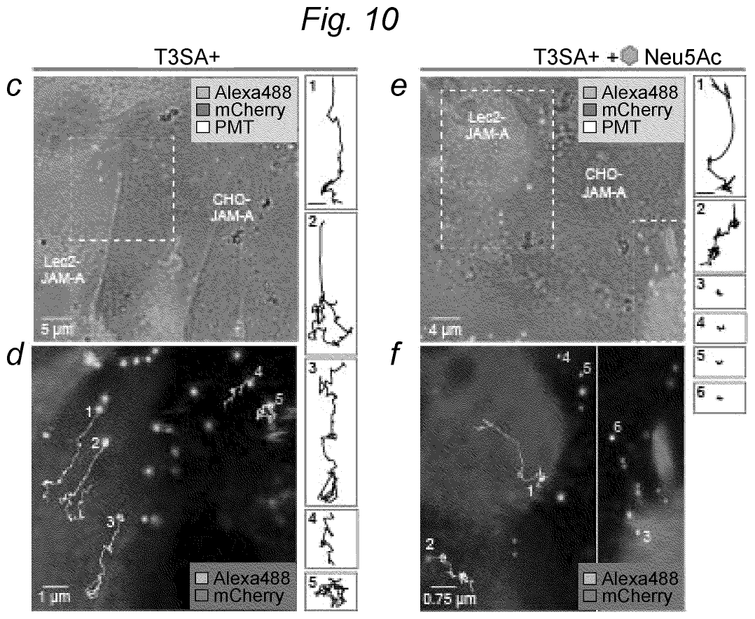

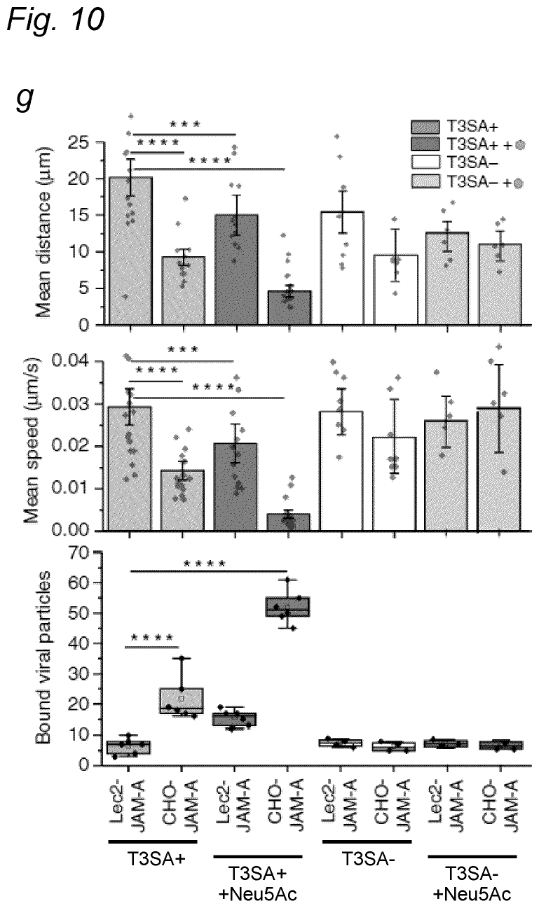

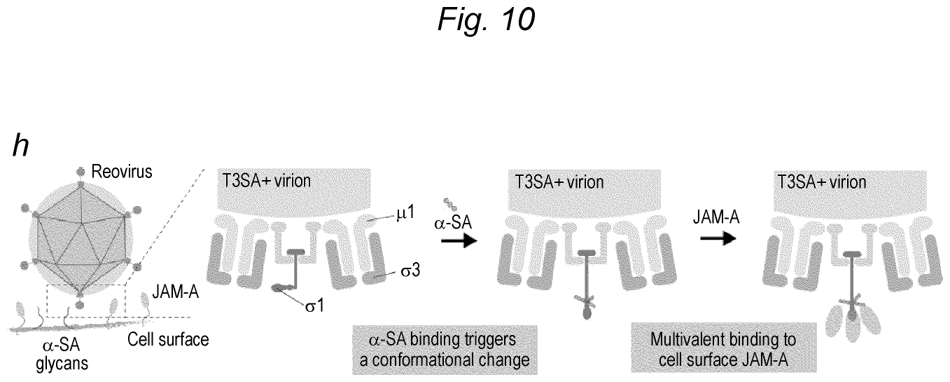

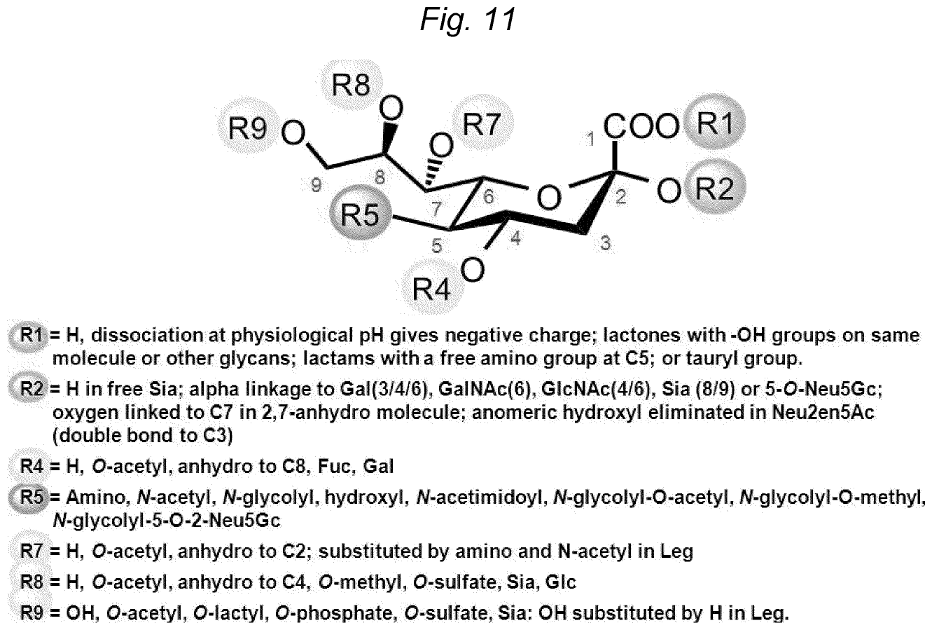

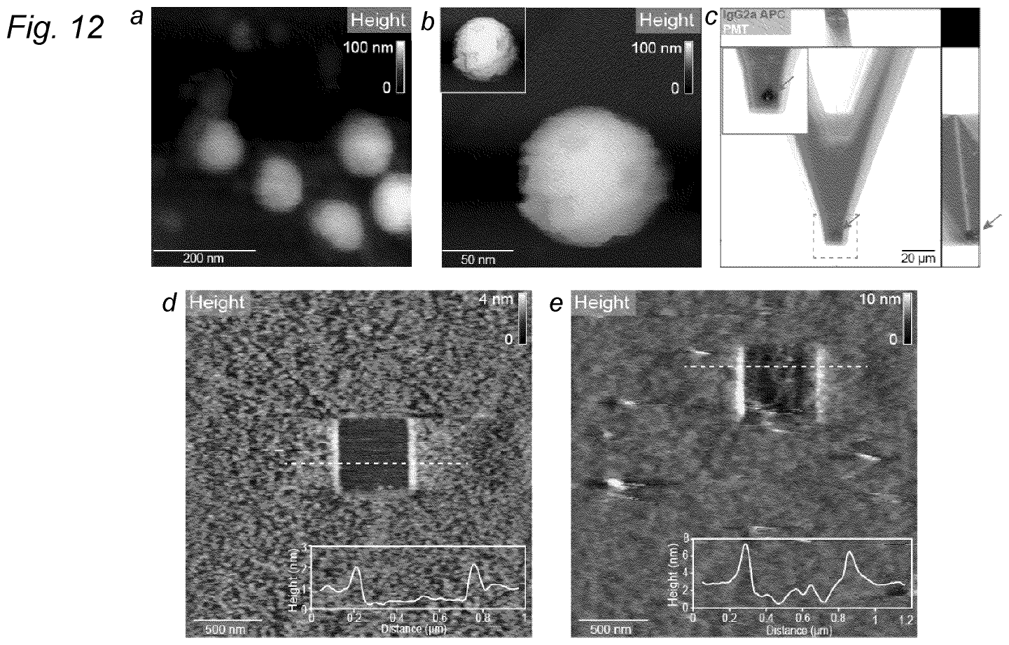

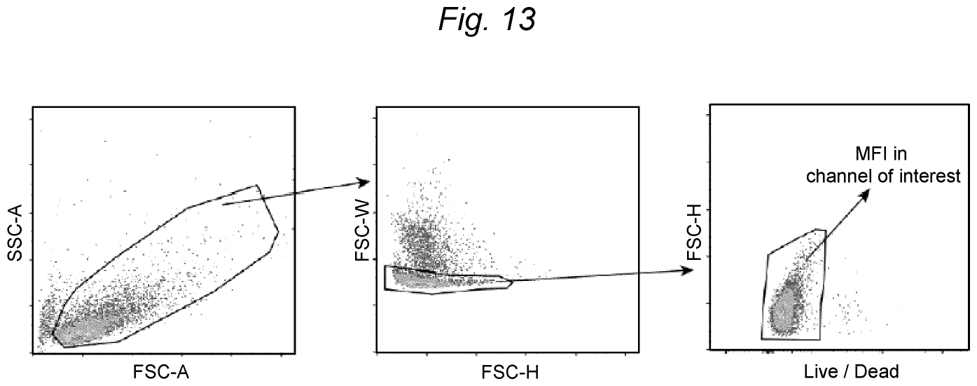

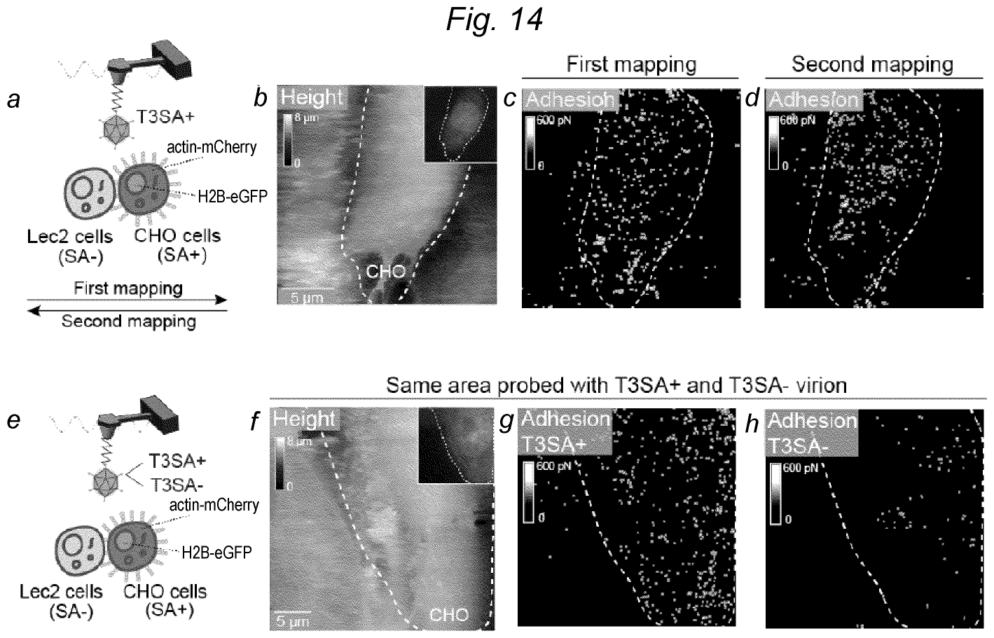

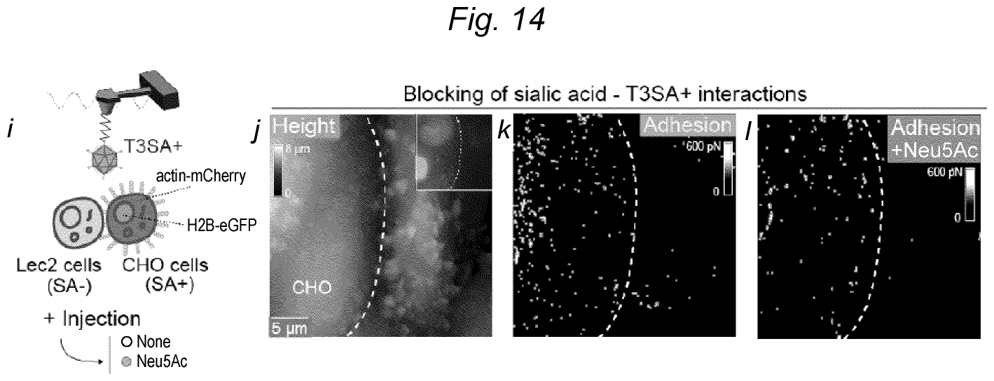

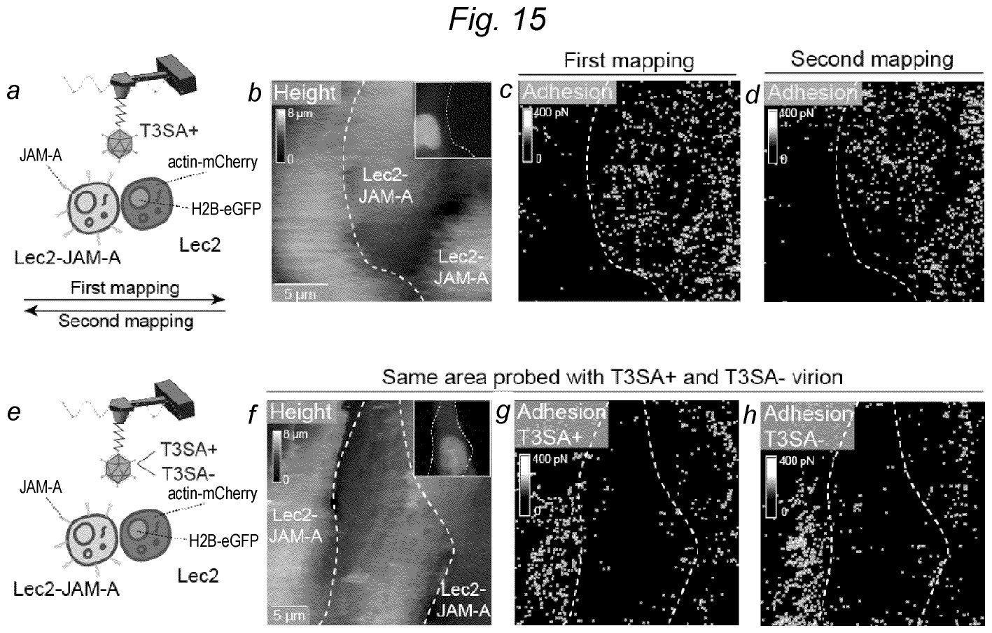

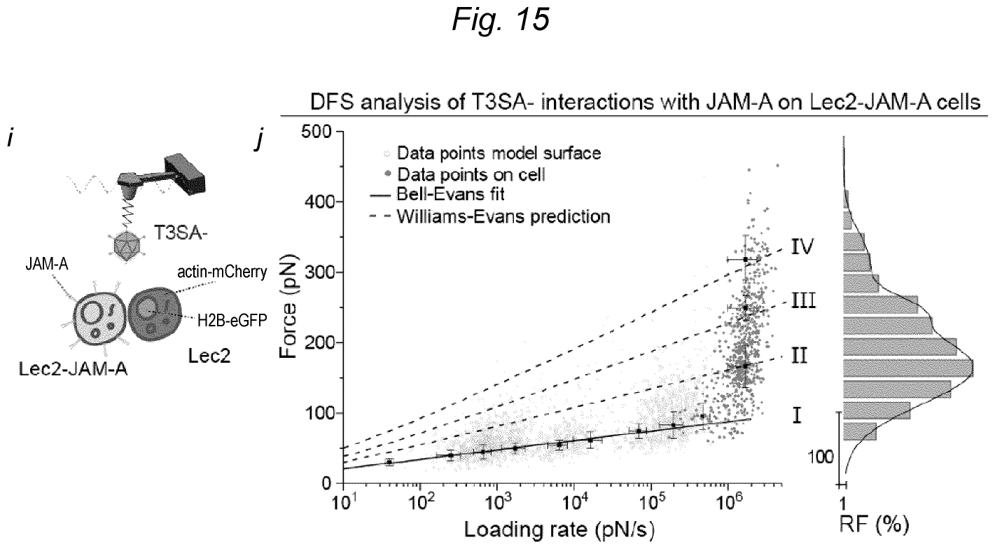

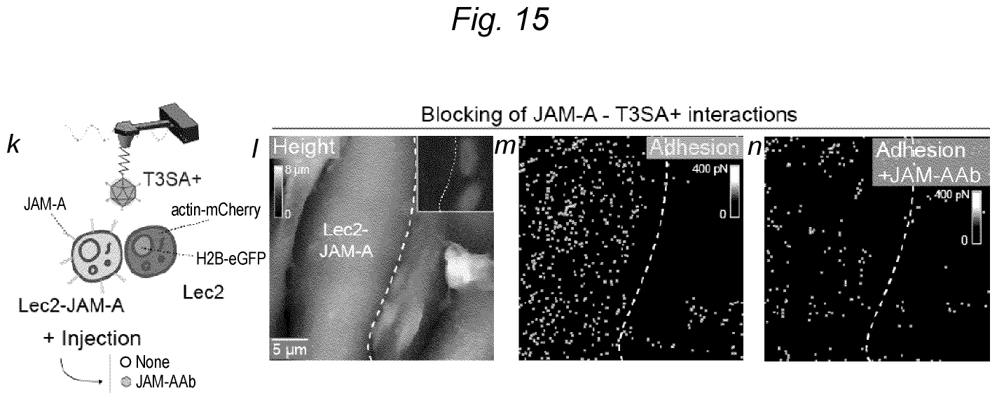

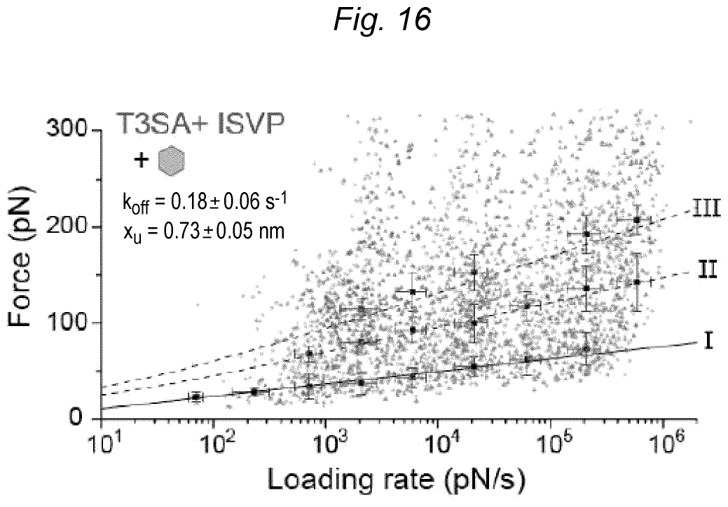

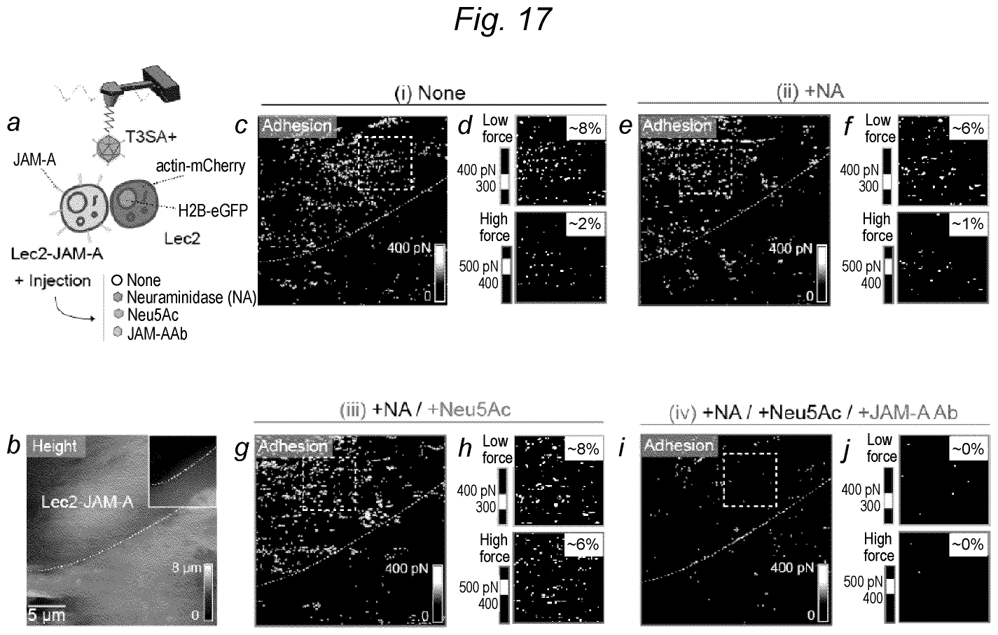

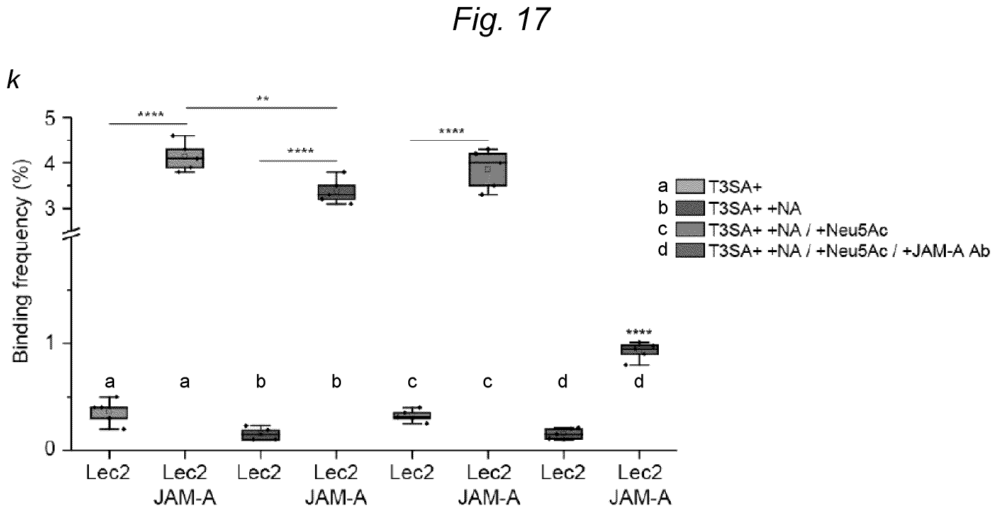

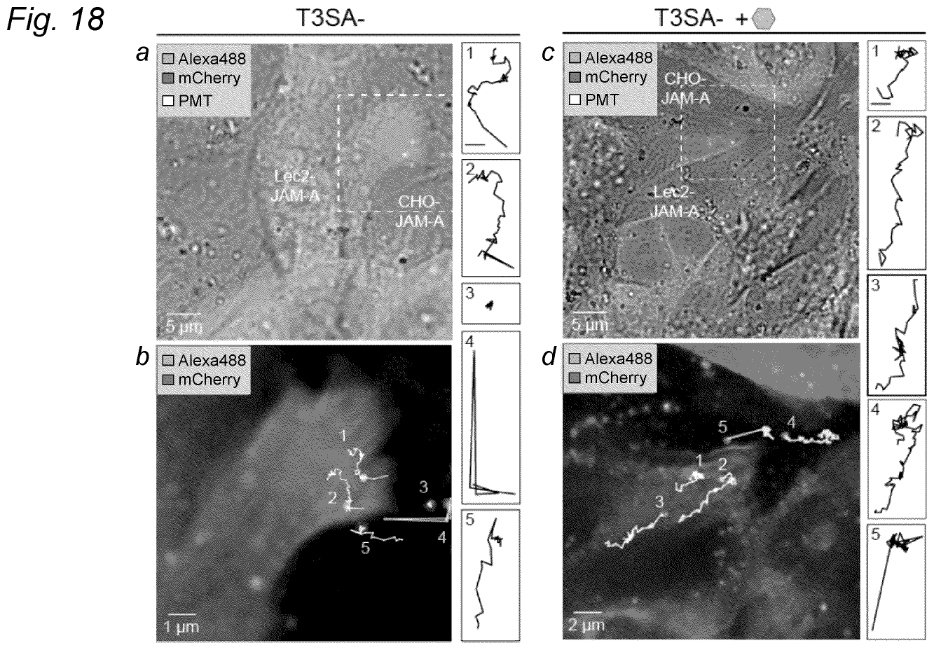

FIG. 17 illustrates monitoring the effect of SA addition on reovirus binding to living cells after neuraminidase treatment. (a) Cartoon of the experiment highlights that Lec2 cells are fluorescently labeled and shows the order of the injections. (b-j) FD-based AFM height image (25 μm×25 μm fluorescent image of the cells is shown in inset) (b) and corresponding adhesion channels, acquired first in growth medium (c), followed by scanning the same area after neuraminidase treatment (e) to remove remaining α-SA on the cell surface. A slight decrease (P<0.01) in adhesion events is observed, indicating that NA treatment removed residual SA on the cell surface. (d,f) Enlarged images of adhesion maps recorded on Lec2-JAM-A cells (dashed square in adhesion map). The upper images display the lower force range (300 to 400 pN), whereas the lower images display the higher force range (400 to 500 pN), with significantly fewer adhesion events before and after NA treatment. The frequency of adhesion events is indicated. After NA treatment, free Neu5Ac (1 mM) was added, and the same area was rescanned (g). Enlarged images of adhesion maps recorded on Lec2-JAMA cells (dashed square in adhesion map and similar areas as in c,e show more adhesion events in the high force range following injection of sialylated glycan. This result is concordant with the experiment conducted using cells without NA treatment (FIG. 8a-e). As a final step, the same area was scanned after injection of 10 μg/ml JAM-A Ab (I, j) to block cell-surface JAM-A molecules. A significant reduction in adhesion events was observed. All AFM images were acquired using an oscillation frequency of 0.25 kHz and amplitude of 750 nm under cell-culture conditions. Experiments were repeated 3-5 times. For clarity and better visibility, the pixel size in the adhesion images were enlarged two-fold. (k) Box plot of the BF observed for T3SA+ virions first without treatment (grey), followed by NA treatment (blue), addition of free Neu5Ac (red), and finally, injection of JAM-A Ab (brown). The horizontal line within the box indicates the median, boundaries of the box indicate the 25^{th} and 75^{th}-percentile, and the whiskers indicate the highest and lowest values of the results. The square in the box indicates the mean. Data are representative of at least n=4 independent experiments. **, P<0.01; ****, P<0.0001; determined by two-sample t-test in Origin.

FIG. 18 illustrates real-time confocal fluorescence imaging of Alexa 488-labeled T3SA− reovirus incubated on co-culture of CHO-JAM-A and Lec2-JAM-A cells in the absence (a, b) and presence (c, d) of 1 mM Neu5Ac. (a, c) Overlay images of Alexa 488 (virions), mCherry (actin of Lec2-Jam-A), and PMT signals. (b, d) Time-lapse trajectories of T3SA particles. White (1 and 2 in b; 1-3 in d) and yellow (3-5 in b; 4-5 in d) trajectories represent virion movement on Lec2-JAM-A cells and CHO-JAM-A cells, respectively. A magnification of each trajectory is shown on the right side with the corresponding number (scale bar: 1 μm). T3SA− particles diffuse on both cell types to a similar extent and independent of the addition of 1 mM Neu5Ac (NeuAc added in right panel), due to the lack of SA binding by T3SA−.

Colored versions of corresponding drawings can also be consulted in Koehler et al. Glycan-mediated enhancement of reovirus receptor binding. Nat Commun. 2019, vol. 10, 4460.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

As corroborated by the experimental section, which illustrates certain representative embodiments of the invention, the inventors for the first time demonstrated that sialic acid (SA) binding to the reovirus sigma 1 (σ1) protein actively promotes a conformational change in the σ1 protein towards a more elongated or extended conformation, which triggers the σ1-binding potential to the JAM-A surface receptor, increases the number of bonds established between the virus and the cell surface favours virus entry into the cytosol. The data thus corroborate the use of sialic acid or sialic acid-containing substances as agents or adjuvants capable of increasing reovirus infectivity.

Accordingly, an aspect of the invention provides a composition or a kit-of-parts comprising i) a virus which is a member of the Reoviridae family and ii) sialic acid and/or a molecule comprising at least one sialic acid moiety. Particularly provided thus are:
a composition comprising, consisting essentially of, or consisting of i) a virus which is a member of the Reoviridae family and ii) sialic acid;
a composition comprising, consisting essentially of, or consisting of i) a virus which is a member of the Reoviridae family and ii) a molecule comprising at least one sialic acid moiety;
a composition comprising, consisting essentially of, or consisting of i) a virus which is a member of the Reoviridae family and ii) sialic acid and a molecule comprising at least one sialic acid moiety;
a kit-of-parts comprising, consisting essentially of, or consisting of i) a virus which is a member of the Reoviridae family and ii) sialic acid;
a kit-of-parts comprising, consisting essentially of, or consisting of i) a virus which is a member of the Reoviridae family and ii) a molecule comprising at least one sialic acid moiety; as well as
a kit-of-parts comprising, consisting essentially of, or consisting of i) a virus which is a member of the Reoviridae family and ii) sialic acid and a molecule comprising at least one sialic acid moiety.

A further aspect provides the composition for use in therapy. A related aspect provides use of the composition in therapy. A further aspect provides the kit-of-parts for use in therapy. A related aspect provides use of the kit-of-parts in therapy. A further aspect provides a method for treating a subject in need thereof, the method comprising administering to the subject a prophylactically or therapeutically effective amount of i) a virus which is a member of the Reoviridae family and ii) sialic acid and/or a molecule comprising at least one sialic acid moiety.

A further aspect provides an in vitro method for propagating a virus which is a member of the Reoviridae family, the method comprising: i) infecting a host cell susceptible to infection by said virus, wherein the host cell has been genetically engineered to overexpress JAM-A, with said virus, either in the presence of sialic acid and/or a molecule comprising at least one sialic acid moiety, or wherein said virus has been previously treated with sialic acid and/or a molecule comprising at least one sialic acid moiety; ii) allowing the virus to propagate in said host cell; and optionally iii) isolating the propagated virus produced by the host cell. Techniques to transiently or stably, constitutively or inducibly, overexpress proteins of interest in host cells are well-known to the skilled person and need not be described in detail. JAM-A protein and nucleic acids encoding it are also well-known. By means of an example, human JAMA mRNA sequence is annotated under NCBI Genbank (http://www.ncbi.nlm.nih.gov/) accession number NM_016946.6. By means of an example, human JAM-A precursor protein sequence is annotated under NCBI Genbank accession number NP_058642.1 and is reproduced below (SEQ ID NO: 1) (amino acids 1 to 27 of SEQ ID NO: 1 have been shown or predicted to constitute a signal peptide processed away from mature JAM-A):

(SEQ ID NO: 1)
MGTKAQVERKLLCLFILAILLCSLALGSVTVHSSEPEVRIPENNPVKL
SCAYSGFSSPRVEWKFDQGDTTRLVCYNNKITASYEDRVTFLPTGITF
KSVTREDTGTYTCMVSEEGGNSYGEVKVKLIVLVPPSKPTVNIPSSAT
IGNRAVLTCSEQDGSPPSEYTWFKDGIVMPTNPKSTRAFSNSSYVLNP
TTGELVFDPLSASDTGEYSCEARNGYGTPMTSNAVRMEAVERNVGVIV
AAVLVTLILLGILVFGIWFAYSRGHFDRTKKGTSSKKVIYSQPSARSE
GEFKQTSSFLV

All JAM-A isoforms are included. By means of an example, an alternative splicing isoform of JAM-A is known lacking amino acids 81-129 of SEQ ID NO: 1, as represented here below (SEQ ID NO: 2):

(SEQ ID NO: 2)
MGTKAQVERKLLCLFILAILLCSLALGSVTVHSSEPEVRIPENNPVKL
SCAYSGFSSPRVEWKFDQGDTTRLVCYNNKITVPPSKPTVNIPSSATI
GNRAVLTCSEQDGSPPSEYTWFKDGIVMPTNPKSTRAFSNSSYVLNPT
TGELVFDPLSASDTGEYSCEARNGYGTPMTSNAVRMEAVERNVGVIVA
AVLVTLILLGILVFGIWFAYSRGHFDRTKKGTSSKKVIYSQPSARSEG
EFKQTSSFLV.

Overexpression encompasses any level of expression above or exceeding the level of JAM-A expression naturally displayed by the host cell, i.e., displayed in the absence of the genetic engineering.

The term “composition” generally refers to a thing composed of two or more components, and more specifically particularly denotes a mixture or a blend of two or more materials, such as elements, molecules, substances, biological molecules, or microbiological materials, as well as reaction products and decomposition products formed from the materials of the composition. Having regard to their usage, the present compositions may be configured as pharmaceutical compositions. Pharmaceutical compositions typically comprise one or more pharmacologically active ingredients (chemically and/or biologically active materials having one or more pharmacological effects) and one or more pharmaceutically acceptable carriers. Compositions as typically used herein may be liquid, semisolid or solid, and may include solutions or dispersions.

The terms “kit” or “kit-of-parts” are interchangeable and denote a combination (combined product) comprising two or more components (more particularly, two or more materials, such as elements, molecules, substances, biological molecules, or microbiological materials, and/or reaction products and decomposition products formed from the materials of the kit) in which one or more components of the combination are kept physically separate (e.g., in separate compartments, containers or vials) from one or more other components of the combination, but adjacent, typically as part of the same product package or dispensing device. Such arrangements allow a consumer or practitioner to admix the components of the kit shortly before use, or to use or administer the physically separated components of the kit separately, such as simultaneously or sequentially in any order.

By means of examples and not limitation, the composition disclosed herein may comprise, consist essentially of, or consist of the Reoviridae virus and the sialic acid and/or molecule comprising at least one sialic acid moiety. The composition may be a pharmaceutical composition also comprising one or more pharmaceutically acceptable carriers. The composition or pharmaceutical composition may be comprised in a kit, physically separated from one or more other components of the kit. By means of examples and not limitation, the kit disclosed herein may comprise the Reoviridae virus and the sialic acid and/or molecule comprising at least one sialic acid moiety, wherein the Reoviridae virus is physically separated from the sialic acid and/or molecule comprising at least one sialic acid moiety. For example, the kit may comprise a composition comprising the Reoviridae virus, physically separated from a composition comprising the sialic acid and/or molecule comprising at least one sialic acid moiety. One or more of the compositions may comprise one or more pharmaceutically acceptable carriers.

In the present context, the compositions or kits-of-parts particularly denote man-made preparations, objects or articles of manufacture. Such compositions or kits-of-parts are particularly useful for example in the medical field such as in therapy. From this perspective, the terms may exclude instances in which a Reoviridae virus and sialic acid or a molecule comprising at least one sialic acid moiety are brought together or brought into proximity merely as part of a contact between the Reoviridae virus and a host cell displaying the sialic acid or the molecule comprising the at least one sialic acid moiety at its cell surface (e.g., included in a glycan decorating a cell surface glycoprotein or ganglioside), whether such contact takes place during a naturally-occurring infection of the host cell by the virus, or is reproduced in a laboratory, such as in cell culture. Hence, in particular embodiments, the composition or the kit-of-parts does not comprise cells, such as in particular host cells of the Reoviridae virus, or cells comprising the cognate cell surface receptor for the Reoviridae virus. In particular embodiments, the composition or the kit-of-parts does not comprise cell membranes, such as in particular cell membranes prepared from host cells of the Reoviridae virus, or from cells comprising the cognate cell surface receptor for the Reoviridae virus. In particular embodiments, the composition or the kit-of-parts does not comprise the cognate cell surface receptor for the Reoviridae virus, and the Reoviridae virus is thus not engaged in an interaction with its cognate receptor, when the virus is part of the composition or the kit-of-parts. In particular embodiments, the sialic acid or the molecule comprising the at least one sialic acid moiety is not associated with or bound to the surface of a cell or cell membrane, for example is not included in a glycan of a glycoprotein or ganglioside on the cell surface (e.g., a transmembrane or extracellular glycoprotein or ganglioside). In particular embodiments, the composition or the kit-of-parts does not comprise a complex composed of the Reoviridae virus bound to a cell or cell membrane, such as wherein the cell or cell membrane contains associated therewith or bound thereto the sialic acid or the molecule comprising the at least one sialic acid moiety, and optionally the cognate cell surface receptor for the Reoviridae virus. In particular embodiments, the composition or the kit-of-parts does not comprise a complex comprising the Reoviridae virus engaged in an interaction with its cognate receptor, when the virus is part of the composition or the kit-of-parts.

The phrases “a virus which is a member of the Reoviridae family” or “Reoviridae family virus” or “Reoviridae virus” encompass any virus that is classified or would be classified in the Reoviridae family following the established practice of virus classification or taxonomy, such as following the International Committee on Taxonomy of Viruses (ICTV) classification system. By means of further guidance and without limitation, Reoviridae viruses are ribonucleic acid (RNA) viruses containing a core of segmented (typically 10-12 segments) double-stranded RNA, lack an outer lipid envelope, and have an icosahedral capsid comprising concentric outer and inner protein shells.

Viruses of any Reoviridae sub-family, including in particular Sedoreovirinae and Spinareovirinae sub-families, are encompassed herein.

Viruses of any Reoviridae genus, including in particular Cardoreovirus, Mimoreovirus, Orbivirus, Phytoreovirus, Rotavirus, Seadornavirus, Aquareovirus, Coltivirus, Cypovirus, Dinovernavirus, Fyivirus, Idnoreovirus, Mycoreovirus, Orthoreovirus, and Oryzavirus genera, are encompassed herein. Without limitation, Orthoreovirus, Orbivirus, Coltivirus, and Rotavirus species are known to infect humans; certain Orthoreovirus species are known to infect birds; Phytoreovirus and Fijivirus species are known to infect plants and insects; Cypovirus species are known to infect insects; and Aquareoviruses are known to infect fish.

Viruses of any Reoviridae species, including in particular Eriocheir sinensis reovirus (Cardoreovirus sp.); Micromonas pusilla reovirus (Mimoreovirus sp.); African horse sickness virus, Bluetongue virus, Changuinola virus, Chenuda virus, Chobar Gorge virus, Corriparta virus, Epizootic hemorrhagic disease virus, Equine encephalosis virus, Eubenangee virus, Great Island virus, leri virus, Lebombo virus, Orungo virus, Palyam virus, Peruvian horse sickness virus, St Croix River virus, Umatilla virus, Wad Medani virus, Wallal virus, Warrego virus, Wongorr virus, Yunnan orbivirus (all Orbivirus sp.); Rice dwarf virus, Rice gall dwarf virus, Wound tumor virus (all Phytoreovirus sp.); Rotavirus A, B, C, D, E, F, G, H, I (all Rotavirus sp.); Banna virus, Kadipiro virus, Liao ning virus (all Seadornavirus sp.); Aquareovirus A, B, C, D, E, F, G (all Aquareovirus sp.); Colorado tick fever virus, Eyach virus (all Coltivirus sp.); Cypovirus 1, 2, 3, 4, 5, 6, 7, 8, 9, 10, 11, 12, 13, 14, 15, 16 (all Cypovirus sp.); Aedes pseudoscutellaris reovirus (Dinovernavirus sp.); Fiji disease virus, Garlic dwarf virus, Maize rough dwarf virus, Mal de Rio Cuarto virus, Nilaparvata lugens reovirus, Oat sterile dwarf virus, Pangola stunt virus, Rice black streaked dwarf virus, Southern rice black-streaked dwarf virus (all Fijivirus sp.); Idnoreovirus 1, 2, 3, 4, 5 (all Idnoreovirus sp.); Mycoreovirus 1, 2, 3 (all Mycoreovirus sp.); Avian orthoreovirus, Baboon orthoreovirus, Mahlapitsi orthoreovirus, Mammalian orthoreovirus, Nelson Bay orthoreovirus, Piscine orthoreovirus, Reptilian orthoreovirus (all Orthoreovirus sp.); Echinochloa ragged stunt virus, Rice ragged stunt virus (all Oryzavirus sp.) are encompassed herein. Viruses of any serotype, strain, clone or isolate within any Reoviridae species are also encompassed herein.

In certain embodiments, the Reoviridae virus displays host tropism for animals. Host tropism refers to the infection specificity of the virus to a particular host, group of hosts, or host taxon(s). A virus may typically specifically infect one or more cell types, tissues or organs of a host (tissue tropism). Hence, a virus may be able to infect animals, but not plants, protists and fungi. In certain embodiments, the Reoviridae virus displays host tropism for at least one animal genus, such as for example for exactly one specific animal genus, or for exactly two or more specific animal genera, or more broadly for a range of animal species or genera.

In certain embodiments, the Reoviridae virus displays host tropism for at least one animal species, such as for example for exactly one specific animal species, or for exactly two or more specific animal species, which typically may but need not belong to the same animal genus, or more broadly for a range of animal species or genera. In certain embodiments, the Reoviridae virus displays host tropism for at least one warm-blooded animal species, such as for example for exactly one specific warm-blooded animal species, or for exactly two or more specific warm-blooded animal species, which typically may but need not belong to the same warm-blooded animal genus, or more broadly for a range of warm-blooded animal species or genera.

In certain embodiments, the Reoviridae virus displays host tropism for at least one vertebrate species, such as for example for exactly one specific vertebrate species, or for exactly two or more specific vertebrate species, which typically may but need not belong to the same vertebrate genus, or more broadly for a range of vertebrate species or genera. The term “vertebrate” broadly encompasses any animal classified within the subphylum Vertebrata following the established taxonomical practice, and by means of illustration includes certain classes of fish, as well as amphibians, reptiles, birds, and mammals.

In certain embodiments, the Reoviridae virus displays host tropism for at least one bird species, such as for example for exactly one specific bird species, or for exactly two or more specific bird species, which typically may but need not belong to the same bird genus, or more broadly for a range of bird species or genera. The term “bird” broadly encompasses any vertebrate animal classified within the class Ayes following the established taxonomical practice. Preferred birds may be fowl, including gamefowl and landfowl (Galliformes) and waterfowl (Anseriformes), such as chickens, quails, turkeys, partridges, pheasants, ducks, geese, or swans.

In certain embodiments, the Reoviridae virus displays host tropism for at least one mammalian species, such as for example for exactly one specific mammalian species, or for exactly two or more specific mammalian species, which typically may but need not belong to the same mammalian genus, or more broadly for a range of mammalian species or genera. The term “mammal” broadly encompasses any vertebrate animal classified within the class Mammalia following the established taxonomical practice, and by means of illustration includes humans, non-human primates, rodents (e.g., mice or rats), canines, felines, equines, ovines, porcines, etc.

In certain embodiments, the Reoviridae virus displays host tropism for humans. Reference herein to any taxon, such as a species, encompasses individuals of that species of any sex or gender (e.g., male or female) and any age.

In certain embodiments, the Reoviridae virus is an Orthoreovirus, such as Avian orthoreovirus, Baboon orthoreovirus, Mahlapitsi orthoreovirus, Mammalian orthoreovirus, Nelson Bay orthoreovirus, Piscine orthoreovirus, or Reptilian orthoreovirus. In certain preferred embodiments, the Reoviridae virus is an Avian orthoreovirus, including any serotypes or strains thereof. Avian orthoreovirus is of particular economic importance owing to its widespread occurrence in poultry flocks.

In certain preferred embodiments, the Reoviridae virus is a Mammalian orthoreovirus. Mammalian orthoreovirus infects numerous mammalian species, including humans. Mammalian or human orthoreovirus is also commonly denoted simply as ‘reovirus’, which is a descriptive acronym for ‘Respiratory and enteric orphan virus’ based on the historic observation that the viruses could be isolated from both the respiratory and enteric tracts of humans although not associated with any known disease state in humans (Sabin. Reoviruses: a new group of respiratory and enteric viruses formerly classified as ECHO type 10 is described. Science. 1959, vol. 130, 1387-1389). Hence, in certain embodiments, the Reoviridae virus is human reovirus. Any serotype or strain of reovirus is encompassed herein. Currently, reovirus includes four known serotypes (or strains), i.e., Type 1 (strain Lang, T1L), Type 2 (strain Jones, T2J), Type 3 (strain Dearing or strain Abney, T3D), and Type 4 (strain Ndelle, T4N). In certain preferred embodiments, the reovirus may be Type 3 reovirus. The serotypes can be distinguished based inter alia on antibody neutralisation and hemagglutinin-inhibition assays as known in the art. Occasionally, the designation ‘reovirus’ may be used in the field to denote other Orthoreovirus species, such as in the phrase “Avian reovirus”.

In certain embodiments, the Reoviridae virus is an Orbivirus, such as African horse sickness virus, Bluetongue virus, Changuinola virus, Chenuda virus, Chobar Gorge virus, Corriparta virus, Epizootic hemorrhagic disease virus, Equine encephalosis virus, Eubenangee virus, Great Island virus, Ieri virus, Lebombo virus, Orungo virus, Palyam virus, Peruvian horse sickness virus, St Croix River virus, Umatilla virus, Wad Medani virus, Wallal virus, Warrego virus, Wongorr virus, or Yunnan orbivirus, including any serotypes or strains thereof. Orbiviruses can infect and replicate within a wide range of arthropod and vertebrate hosts, including without limitation cattle, goats and sheep, wild ruminants, equids, camelids, marsupials, sloths, bats, birds, large canine and feline carnivores, and humans. In certain preferred embodiments, the Orbivirus is Bluetongue virus, African horse sickness virus, or Epizootic hemorrhagic disease virus, including any serotypes or strains thereof, which are of particular economic importance owing to their occurrence economically important animals, such as such as sheep, cattle, buffalo, deer, horses, mules and donkeys.

In certain embodiments, the Reoviridae virus is a Rotavirus, such as Rotavirus species A, B, C, D, E, F, G, H, I, including any serotypes or strains thereof, which constitute the most common cause of diarrhoeal disease among infants and young children. In certain preferred embodiments, the Rotavirus is Rotavirus A, including any serotypes or strains thereof, which is the most common species, causing more than 90% of rotavirus infections in humans.

Whereas reference to a virus as used herein may encompass the virus at any stage of its lifecycle, and in any shape or form occurring in the course of its lifecycle, particularly intended by the term are virus particles or virions, more particularly intact virus particles or virions.

The Reoviridae virus may be naturally occurring or non-naturally occurring. The virus can be considered as “naturally occurring” when it has been isolated from a source in nature, and optionally propagated in a suitable biological system (such as in cultured cell lines susceptible to the infection by the virus) and collected, enriched or purified, but has not been intentionally modified by the hand of man. For example, the virus may have been isolated from a field source, such as a host individual, for example a human individual, who has been infected with the virus. The virus may be culture-adapted. The virus can be considered as “non-naturally occurring” when it has been modified compared to the corresponding naturally occurring virus. Such modification may include chemical or biochemical treatments which substantially alter the structure of the virus, such as without limitation connect a detectable label to the outer capsid, proteolytically truncate or remove one or more components of the outer capsid, or coat the virus in a liposome or micelle, and/or may include genetic engineering of the viral nucleic acids. Genetic engineering may alter one or more viral genes and/or nucleic acids surrounding the one or more viral genes, and may affect viral processes, such as, for example, viral infectivity, viral DNA replication, viral protein synthesis, virus particle assembly and maturation, and viral particle release, or may introduce a site for insertion into the virus of heterologous nucleic acids. Such heterologous nucleic acid(s) may for example but without limitation include genetic payload deleterious or toxic to host cells, e.g., to further stimulate the toxicity of an oncolytic form of the virus towards neoplastic cells. For example, introducing a gene encoding an inducer, mediator or executioner of apoptosis, such as TNF-related apoptosis inducing ligand (TRAIL), interleukin-24, a caspase, or an siRNA or microRNA silencer of an endogenous anti-apoptotic gene, may be an option. The virus can also be considered as “non-naturally occurring” when obtained by recombination of two or more subtypes of a Reoviridae virus species, such as two or more reovirus subtypes (recombinant virus), with differing pathogenic phenotypes, such that it contains different antigenic determinants, thereby reducing or preventing an immune response by a host previously exposed to a Reoviridae virus, such as a mammal previously exposed to a reovirus subtype. Such recombinant virions can be generated by co-infection of host cells with different subtypes of the Reoviridae virus, such as different subtypes of reovirus, with the resulting resorting and incorporation of different subtype coat proteins into the resulting virion capsids.

The Reoviridae virus as intended herein may particularly be viable or live virus, in the sense that the virus is capable of infecting a host cell, such as an in vitro cultured host cell, susceptible to infection by said virus (such cells typically express a cognate surface receptor for the virus and are permissive for the virus replication). Such infection typically involves several stages or steps, including attachment of the virus particles to cognate receptors at the host cell surface, their uptake, intracellular trafficking, and penetration to the cytosol, uncoating, replication of the viral nucleic acids and production of viral proteins, and assembly and release of the newly produced virus particles. In certain embodiments, the virus may infect the host cell without lysing the host cell (non-lytic infection). In certain embodiments, the infection of the host cell with the virus may lead to lysis of the host cell (lytic infection). In other words, such Reoviridae virus is not rendered non-viable, inactivated or killed.

A Reoviridae virus may be isolated from a field source, such as from a biological sample of a host infected with the virus. Depending on the tissue tropism of the virus, virus particles may shed into and be recovered from a variety of biological samples, which may include organ or tissue specimens, whole blood, plasma, lymph, serum, blood cells, saliva, urine, stool (feces), tears, sweat, sebum, nipple aspirate, ductal lavage, synovial fluid, cerebrospinal fluid, amniotic fluid, semen, vaginal secretions, inflammation fluid, or any other bodily fluids, exudates or secretory fluids. By means of an example, the sample may be homogenised where necessary using standard techniques of tissue homogenisation, such as mincing and blending in a suitable buffer, debris may be pelleted by centrifugation, and the virus-containing supernatant may be collected and passed through a 0.45 μm or 0.25 μm, which separates cells and allows the virus to pass. The resulting filtrate may be used to inoculate a suitable cultured cell line susceptible to infection by the virus, in suspension or in monolayer culture, in order to propagate the virus. By means of an example, mammalian reoviruses may be typically cultured using mouse fibroblast L929 cell line (available inter alia from European Collection of Cell Cultures, ECACC, Health Protection Agency—Porton Down Salisbury, Wiltshire SP4 OJG, United Kingdom, cat. no. 85011425). See also Berard & Coombs. Mammalian reoviruses: propagation, quantification, and storage. Curr Protoc Microbiol. 2009 Chapter 15: Unit 15C.1. The propagated virus may be purified from the infected cell lysates by caesium chloride gradient centrifugation for further use. The term “purified” in this context does not require absolute purity. Instead, it denotes that the thing that has been purified is in a discrete environment in which its abundance relative to other components is greater than in the original material. A discrete environment denotes a single medium, such as for example a single solution, gel, precipitate, lyophilisate, etc. Subsequent to purification, viral proteins or polypeptides may preferably constitute by weight ≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the protein content of the discrete environment. Protein content may be determined, e.g., by the Lowry method (Lowry et al. 1951 J Biol Chem 193:265), optionally as described by Hartree 1972 Anal Biochem 48:422-427. Purity of peptides, polypeptides, or proteins may be determined by SDS-PAGE under reducing or non-reducing conditions using Coomassie blue or, preferably, silver stain. Viral infectivity of the virus may be ascertained by determining the viral titre using standard techniques, such as a plaque assay or through calculating the infectious dose, or determination of virus loads in a challenged host. Where necessary, the virus may be preserved by standard procedures, such as cryopreservation using common cryopreservants, such as glycerol or DMSO, or lyophilisation (freeze-drying) using common stabilisers, such as glucose, skim milk, or Sucrose-Phosphate-Glutamate-Albumin (SPGA). Virus authentication may also employ standard techniques, such as sequencing, immunological assay methods such as ELISA to detect a characteristic surface antigen, etc.

Alternatively, Reoviridae viruses may be obtained from public collections maintained for example by American Type Culture Collection (ATCC) (10801 University Blvd. Manassas, Va. 20110-2209, USA) or by National Collection of Pathogenic Viruses (NCPV) (Public Health England—Porton Down Salisbury, Wiltshire SP4 OJG, United Kingdom), including without limitation, Mammalian reoviruses ATCC acc. no. VR-215, VR-231, VR-232, VR-824, VR-871, or NCPV catalogue number 0006252v, or Avian orthoreovirus ATCC acc. no. VR-826, VR-857, VR-2449, PTA-47.

Intact Reoviridae viruses typically comprise two concentric capsids, albeit the terms used to denote these structures may vary (e.g., outer capsid and inner capsid; outer capsid and inner core; outer shell and inner shell). Cypovirus and Dinovernavirus genera are exceptions as they contain a single capsid. Moreover, some genera, such as Rotavirus and Orbivirus, may be described as additionally containing an intermediate capsid interposed between the outer and inner capsid.

By means of an example, in Orthoreovirus species, including Avian and Mammalian orthoreovirus, the inner capsid is formed by inner capsid proteins lambda 1 and sigma 2, and the outer capsid is formed by outer capsid proteins lambda 2, mu 1, sigma 1 and sigma 3. Protease treatment of reovirus, such as by chymotrypsin, has been known to generate infectious subvirion particles (ISVPs) by removal of sigma 3, cleavage of mu 1 to yield delta and phi, and rearrangement of sigma 1 into a more elongated conformation. In certain preferred embodiments, the Reoviridae virus, such as without limitation Orthoreovirus, such as without limitation Avian or Mammalian orthoreovirus, comprises an outer capsid and an inner core. For example, such viruses have not been subjected to protease treatment to generate ISVP.

Without wishing to be bound by any theory, the inventors discovered that sialic acid interaction with the reovirus outer capsid protein sigma 1 (σ1) protein actively promotes a conformational change in the σ1 protein towards a more elongated or extended conformation, which advantageously results in an increased ability of the virus to bind the cognate cell surface receptors, and consequently to infect the cell.

Accordingly, in certain embodiments, the Reoviridae virus comprises an outer capsid protein capable of binding to a host cell surface receptor, wherein the sialic acid or the molecule comprising the at least one sialic acid moiety causes said outer capsid protein to adopt a more elongated or extended conformation on the Reoviridae virus compared to the conformation in the absence of the sialic acid or the molecule comprising the at least one sialic acid moiety. In this context, the phrase “capable of binding to a host cell surface receptor” denotes the specific interaction between an outer capsid protein and its cognate cell surface receptor. The occurrence of such comparatively elongated or extended conformation of the protein can be determined for example using suitable virus visualisation methods, such as X-ray crystallography, cryo-electron microscopy (cryo-EM), and/or atomic force microscopy (AFM) for example as illustrated in the Examples.

Accordingly, in certain embodiments, the Reoviridae virus comprises an outer capsid protein capable of binding to a host cell surface receptor, wherein the sialic acid or the molecule comprising the at least one sialic acid moiety causes said outer capsid protein to bind more strongly to the host cell surface receptor compared to the binding in the absence of the sialic acid or the molecule comprising the at least one sialic acid moiety. The strength of binding can be determined for example using atomic force microscopy (AFM) as employed in the Examples.

In preferred embodiments, the outer capsid protein is sigma-1 protein. In certain preferred embodiments, the Reoviridae virus is an Orthoreovirus, such as without limitation Avian or Mammalian orthoreovirus, comprising outer capsid protein sigma-1 capable of binding to a host cell surface receptor (such as a junctional adhesion molecule (JAM) protein, and more particularly the JAM-A protein, recognised by reovirus), wherein the sialic acid or the molecule comprising the at least one sialic acid moiety causes said sigma-1 protein to adopt a more elongated or extended conformation on the virus compared to the conformation in the absence of the sialic acid or the molecule comprising the at least one sialic acid moiety. In certain preferred embodiments, the Reoviridae virus is an Orthoreovirus, such as without limitation Avian or Mammalian orthoreovirus, comprising outer capsid protein sigma-1 capable of binding to a host cell surface receptor (such as a JAM protein, and more particularly the JAM-A protein, recognised by reovirus), wherein the sialic acid or the molecule comprising the at least one sialic acid moiety causes said sigma-1 protein to bind more strongly to the host cell surface receptor compared to the binding in the absence of the sialic acid or the molecule comprising the at least one sialic acid moiety.

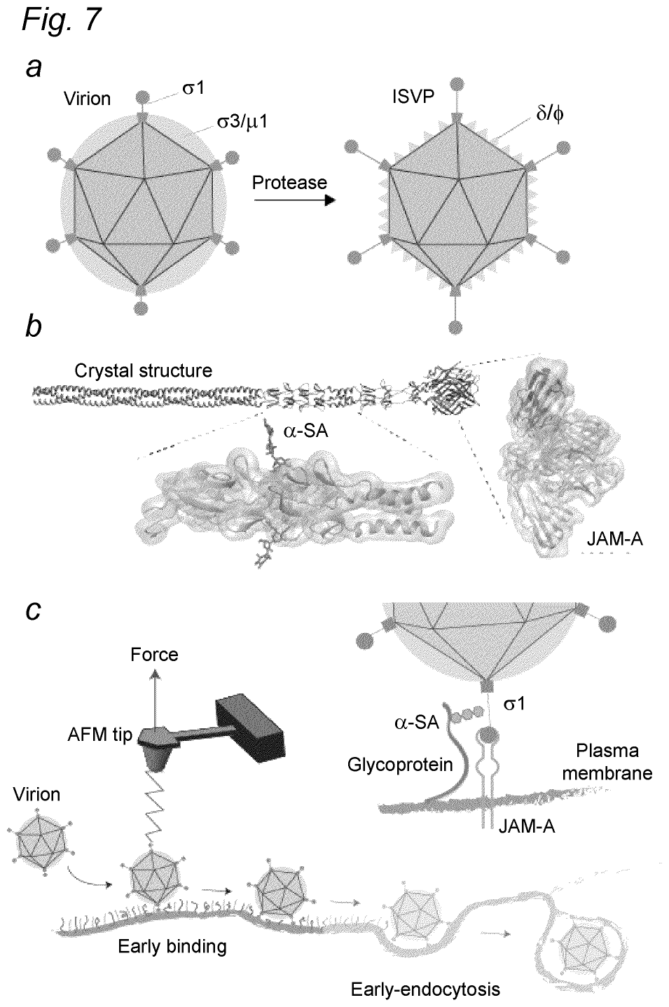

As illustrated in FIG. 7 and discussed elsewhere in this specification, a reovirus σ1 protein may comprise a tail domain, such as in particular formed by α-helical coiled coil and triple-β spiral, and a head domain, such as in particular formed by a compact eight-stranded β-barrel. While tail domain, in particular the triple-β spiral, may bind to α-SA, and the head domain may bind to JAM-A. Hence, in certain embodiments, the σ1 protein as intended herein may comprise a tail domain capable of binding to α-SA and a head domain capable of binding to JAM-A.

In certain embodiments, the Reoviridae virus is an oncolytic virus. The term “oncolytic virus” broadly refers to a virus capable of selectively replicating in dividing cells, more preferably in neoplastic cells (e.g., tumor cells, cancer cells), with the aim of slowing the growth and/or lysing said cells, either in vitro or in vivo, while showing no or minimal replication in non-dividing cells, more preferably in non-neoplastic cells. A preferred example of an oncolytic virus is Mammalian orthoreovirus type 3, such as Mammalian orthoreovirus 3 Dearing, which induces cell lysis and death preferentially in transformed cells and therefore displays inherent oncolytic properties. More particularly, Reovirus Type 3 Dearing is capable of replicating in transformed cells with an activated Ras signalling pathway, whereas normal, untransformed cells are unable to support the infection. Hence, in certain embodiments, neoplastic cells susceptible to infection by oncolytic Reoviridae virus as intended herein may comprise or be characterised by constitutive ras-MAP signalling.

Thus, in certain embodiments, the Reoviridae virus is oncolytic Mammalian orthoreovirus type 3, more preferably type 3 Dearing. By means of example and without limitation, one embodiment of an oncolytic reovirus is manufactured under the trademark Reolysin® by Oncolytics Biotech Inc. (Calgary, Alberta, Canada), particularly indicated for solid tumors and hematological malignancies. REOLYSIN is a non-pathogenic, proprietary isolate of the unmodified reovirus that: induces selective tumor lysis and promotes an inflamed tumor phenotype through innate and adaptive immune responses, as conceived of for example in WO 2000/050051.

Hence, in certain embodiments, envisaged herein is the use of, or a method of using, sialic acid and/or a molecule comprising at least one sialic acid moiety, as an adjuvant to the Reoviridae virus, such as the oncolytic Reoviridae virus, as envisaged herein, to enhance the virus infectivity.

In certain embodiments, the oncolytic Reoviridae virus may be co-administered with a binding agent capable of specifically binding to neoplastic cells, such as co-administered in the same composition, or co-administered from separate compositions simultaneously or sequentially in any order. In certain preferred embodiments, the oncolytic Reoviridae virus may be linked, such as covalently or non-covalently linked, preferably covalently linked, to a binding agent capable of specifically binding to neoplastic cells. In certain embodiments, a non-covalent linkage may involve providing the Reoviridae virus and the binding agent each with a different half or component of an affinity pair, such as without limitation biotin-streptavidin affinity pair, or antibody-hapten affinity pair. For example, streptavidin may be attached, typically covalently attached, to the Reoviridae virus, and biotin may be attached, typically covalently attached, to the binding agent, or vice versa.

The term “specifically bind” means that an agent (denoted herein also as “binding agent” or “specific-binding agent”) binds to one or more desired targets (e.g., peptides, polypeptides, proteins, nucleic acids, or cells) substantially to the exclusion of other entities which are random or unrelated, and optionally substantially to the exclusion of other molecules that are structurally related. The term “specifically bind” does not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold, or at least about 1000-fold, or at least about 10⁴-fold, or at least about 10⁵-fold, or at least about 10⁶-fold or more greater, than its affinity for a non-target. Preferably, the specific binding agent may bind to its intended target(s) with affinity constant (K_{A}) of such binding K_{A}≥1×10⁶ M⁻¹, more preferably K_{A}≥1×10⁷ M⁻¹, yet more preferably K_{A}≥1×10⁸ M⁻¹, even more preferably K_{A}≥1×10⁹ M⁻¹, and still more preferably K_{A}≥1×10¹⁰ M⁻¹ or K_{A}≥1×10¹¹ M⁻¹ or K_{A}≥1×10¹² M⁻¹, wherein K_{A}=[SBA_T]/[SBA][T], SBA denotes the specific-binding agent, T denotes the intended target. Determination of K_{A} can be carried out by methods known in the art, such as for example, using equilibrium dialysis and Scatchard plot analysis.

In certain embodiments, the binding agent may be an antibody. As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

In certain embodiments, the agent may be a Nanobody®. The terms “Nanobody®” and “Nanobodies®” are trademarks of Ablynx NV (Belgium). The term “Nanobody” is well-known in the art and as used herein in its broadest sense encompasses an immunological binding agent obtained (1) by isolating the V_{HH} domain of a heavy-chain antibody, preferably a heavy-chain antibody derived from camelids; (2) by expression of a nucleotide sequence encoding a V_{HH} domain; (3) by “humanization” of a naturally occurring V_{HH} domain or by expression of a nucleic acid encoding a such humanized V_{HH} domain; (4) by “camelization” of a V_{H} domain from any animal species, and in particular from a mammalian species, such as from a human being, or by expression of a nucleic acid encoding such a camelized V_{H} domain; (5) by “camelization” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna).

For example, the binding agent, such as antibody, may be configured to specifically bind a protein expressed by neoplastic cells, such as a tumor antigen. The term “tumor antigen” refers to an antigen that is uniquely or differentially expressed by a tumor cell, whether intracellular or on the tumor cell surface (preferably on the tumor cell surface), compared to a normal or non-neoplastic cell. By means of example, a tumor antigen may be present in or on a tumor cell and not typically in or on normal cells or non-neoplastic cells (e.g., only expressed by a restricted number of normal tissues, such as testis and/or placenta), or a tumor antigen may be present in or on a tumor cell in greater amounts than in or on normal or non-neoplastic cells, or a tumor antigen may be present in or on tumor cells in a different form than that found in or on normal or non-neoplastic cells. The term thus includes tumor-specific antigens (TSA), including tumor-specific membrane antigens, tumor-associated antigens (TAA), including tumor-associated membrane antigens, embryonic antigens on tumors, growth factor receptors, growth factor ligands, etc. The term further includes cancer/testis (CT) antigens. Examples of tumor antigens include, without limitation, β-human chorionic gonadotropin (βHCG), glycoprotein 100 (gp100/Pme117), carcinoembryonic antigen (CEA), tyrosinase, tyrosinase-related protein 1 (gp75/TRP1), tyrosinase-related protein 2 (TRP-2), NY-BR-1, NY-CO-58, NY-ESO-1, MN/gp250, idiotypes, telomerase, synovial sarcoma X breakpoint 2 (SSX2), mucin 1 (MUC-1), antigens of the melanoma-associated antigen (MAGE) family, high molecular weight-melanoma associated antigen (HMW-MAA), melanoma antigen recognized by T cells 1 (MART1), Wilms' tumor gene 1 (WT1), HER2/neu, mesothelin (MSLN), alphafetoprotein (AFP), cancer antigen 125 (CA-125), and abnormal forms of ras or p53. Further targets in neoplastic diseases include without limitation CD37 (chronic lymphocytic leukemia), CD123 (acute myeloid leukemia), CD30 (Hodgkin/large cell lymphoma), MET (NSCLC, gastroesophageal cancer), IL-6 (NSCLC), and GITR (malignant melanoma).

In certain preferred embodiments, the sialic acid and/or the molecule comprising the at least one sialic acid moiety may be linked, such as covalently or non-covalently linked, preferably covalently linked, to said binding agent. In certain embodiments, a non-covalent linkage may involve providing the sialic acid and/or the molecule comprising the at least one sialic acid moiety and the binding agent each with a different half or component of an affinity pair, such as biotin-streptavidin affinity pair, or antibody-hapten affinity pair. For example, streptavidin may be attached, typically covalently attached, to the sialic acid and/or the molecule comprising the at least one sialic acid moiety, and biotin may be attached, typically covalently attached, to the binding agent, or vice versa. This facilitates the interaction between the sialic acid and/or the molecule comprising the at least one sialic acid moiety and the Reoviridae virus linked to said binding agent. In certain preferred embodiments, the oncolytic Reoviridae virus is linked to a binding agent capable of specifically binding to neoplastic cells and the sialic acid and/or the molecule comprising the at least one sialic acid moiety is also linked to said binding agent. In certain preferred embodiments, the oncolytic Reoviridae virus is linked to an antibody capable of specifically binding to neoplastic cells, and the sialic acid and/or the molecule comprising the at least one sialic acid moiety is also linked to said antibody.

Any covalent linkage between two molecules as intended here may be direct or may be via a suitable linker, as generally known in the art, the nature and structure of which is not particularly limited. A linker may be, for example, a peptide or non-peptide linker, such as a non-peptide polymer, such as a non-biological polymer. Preferably, any linkages may be hydrolytically stable linkages, i.e., substantially stable in water at useful pH values, including in particular under physiological conditions, for an extended period of time, e.g., for days.

In certain embodiments, a non-peptide linker may comprise, consist essentially of or consist of a non-peptide polymer. The term “non-peptide polymer” broadly refers to a biocompatible polymer including two or more repeating units linked to each other by a covalent bond excluding the peptide bond. For example, the non-peptide polymer may be 2 to 200 units long or 2 to 100 units long or 2 to 50 units long or 2 to 45 units long or 2 to 40 units long or 2 to 35 units long or 2 to 30 units long or 5 to 25 units long or 5 to 20 units long or 5 to 15 units long. The non-peptide polymer may be selected from the group consisting of polyethylene glycol, polypropylene glycol, copolymers of ethylene glycol and propylene glycol, polyoxyethylated polyols, polyvinyl alcohol, polysaccharides, dextran, polyvinyl ethyl ether, biodegradable polymers such as PLA (poly(lactic acid) and PLGA (polylactic-glycolic acid), lipid polymers, chitins, hyaluronic acid, and combinations thereof. Particularly preferred is poly(ethylene glycol) (PEG). The molecular weight of the non-peptide polymer preferably may range from 1 to 100 kDa, and preferably 1 to 20 kDa. The non-peptide polymer may be one polymer or a combination of different types of polymers. The non-peptide polymer has reactive groups capable of binding to the entities linked thereby. Preferably, the non-peptide polymer has a reactive group at each end. Preferably, the reactive group is selected from the group consisting of a reactive aldehyde group, a propione aldehyde group, a butyl aldehyde group, a maleimide group and a succinimide derivative. The succinimide derivative may be succinimidyl propionate, hydroxy succinimidyl, succinimidyl carboxymethyl or succinimidyl carbonate.

The composition or kit-of-parts comprising the Reoviridae virus, such as the oncolytic Reoviridae virus, and the sialic acid and/or the molecule comprising at least one sialic acid moiety as taught herein, is useful for therapy, and particularly useful in the treatment of neoplastic diseases. Hence, an aspect provides the composition or kit-of-parts comprising the Reoviridae virus, such as the oncolytic Reoviridae virus, and the sialic acid and/or the molecule comprising at least one sialic acid moiety as taught herein, for use in therapy.

A further aspect provides the composition or kit-of-parts comprising the Reoviridae virus, such as particularly the oncolytic Reoviridae virus, and the sialic acid and/or the molecule comprising at least one sialic acid moiety as taught herein, for use in a method of treating a neoplastic disease. A related aspect provides a method of treating a neoplastic disease in a subject, comprising administering to the subject a therapeutically or prophylactically effective amount of the Reoviridae virus, such as particularly the oncolytic Reoviridae virus, and the sialic acid and/or the molecule comprising at least one sialic acid moiety as taught herein. In certain embodiments, the neoplastic disease may be characterised by dysregulation of the ras-MAP signalling pathway, such as by the presence of constitutive ras-MAP signalling. As set forth throughout the specification, the inventors demonstrated that sialic acid (SA) binding to the reovirus sigma 1 (σ1) protein acts as a trigger of σ1-binding potential to the JAM-A cell surface receptor, which is a key step in viral entry into cells. JAM-A can be relatively widely expressed in many cell types, and hence will also be expressed by neoplastic cells, such as tumour or cancer cells, of various tissue origins. The skilled person would immediately appreciate that neoplastic diseases in which at least some neoplastic cells express JAM-A protein are particularly contemplated, as these will particularly benefit to at least a certain degree from the effects and mechanisms described herein.

Reference to “therapy” or “treatment” broadly encompasses both curative and preventative treatments, and the terms may particularly refer to the alleviation or measurable lessening of one or more symptoms or measurable markers of a pathological condition such as a disease or disorder. The terms encompass primary treatments as well as neo-adjuvant treatments, adjuvant treatments and adjunctive therapies. The terms “treating a neoplastic disease” or “anti-cancer therapy” or “anti-cancer treatment” broadly refer to the alleviation or measurable lessening of one or more symptoms or measurable markers of a neoplastic disease. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments as appropriate in the context of the present invention. By means of an example, an ex vivo treatment to remove neoplastic cells from a cellular composition obtained form a subject and/or intended for being introduced or transplanted into a subject using the present compositions or kits-of-parts is encompassed.

The terms “subject”, “individual” or “patient” are used interchangeably throughout this specification, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, buffalo, deer, horses, mules and donkeys, and non-mammals such as birds, chickens, including chickens, quails, turkeys, partridges, pheasants, ducks, geese, or swans, amphibians, reptiles etc. In certain embodiments, the subject is a non-human mammal. In certain preferred embodiments, the subject is human. In certain preferred embodiments, the subject is chicken. In other embodiments, the subject is an experimental animal or animal substitute as a disease model. The term does not denote a particular age or sex. Thus, adult and newborn subjects, as well as fetuses, whether male or female, are intended to be covered. The term subject is further intended to include transgenic non-human species.

The term “therapeutically effective amount” generally denotes an amount sufficient to elicit the pharmacological effect or medicinal response in a subject that is being sought by a medical practitioner such as a medical doctor, clinician, surgeon, veterinarian, or researcher, which may include inter alia alleviation of the symptoms of the disease being treated, in either a single or multiple doses. The term “prophylactically effective amount” generally denotes an amount sufficient to elicit the preventative effect, such as inhibition or delay of the onset of a disease, in a subject that is being sought by the medical practitioner, in either a single or multiple doses. Appropriate prophylactically or therapeutically effective doses of the present compositions or components of the kits-of-parts may be determined by a qualified physician with due regard to the nature and severity of the disease, and the age and condition of the patient. The effective amount of the compositions or components of the kits-of-parts described herein to be administered can depend on many different factors and can be determined by one of ordinary skill in the art through routine experimentation. Several non-limiting factors that might be considered include biological activity of the active ingredient, nature of the active ingredient, characteristics of the subject to be treated, etc. The term “to administer” generally means to dispense or to apply, and typically includes both in vivo administration and ex vivo administration to a tissue, preferably in vivo administration. Generally, compositions may be administered systemically or locally.

The term “neoplastic disease” generally refers to any disease or disorder characterized by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumors, premalignant or precancerous lesions, malignant tumors, and cancer. Examples of neoplastic diseases or disorders are benign, pre-malignant, or malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the terms “tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “cancer” refers to a malignant neoplasm characterized by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis.

Examples of cancer include but are not limited to carcinoma, lymphoma, blastoma, sarcoma, and leukemia or lymphoid malignancies. More particular examples of such cancers include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as CNS cancer, melanoma, head and neck cancer, bone cancer, bone marrow cancer, duodenum cancer, esophageal cancer, thyroid cancer, or hematological cancer.

Other non-limiting examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Non-melanoma Skin Cancer, Non-Small Cell Lung Cancer, Occult Primary Metastatic Squamous Neck Cancer, Oropharyngeal Cancer, Osteo-/Malignant Fibrous Sarcoma, Osteo sarcoma/Malignant Fibrous Histiocytoma, Osteosarcoma/Malignant Fibrous Histiocytoma of Bone, Ovarian Epithelial Cancer, Ovarian Germ Cell Tumour, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, or Wilms' Tumour.

In certain embodiments, the tumor is a solid tumor. Solid tumors encompass any tumors forming a neoplastic mass that usually does not contain cysts or liquid areas. Solid tumors may be benign, pre-malignant or malignant. Examples of solid tumors are carcinomas, sarcomas, melanomas and lymphomas. In certain embodiments, the neoplastic disease may be a hematological malignancy. In certain embodiments, the neoplastic disease may be leukemia. In certain preferred embodiments, the neoplastic disease is malignant glioma.

In certain embodiments, the present compositions or kits-of-parts may be employed in combination with one or more other anti-cancer therapy (combination therapy). Non-limiting examples of anti-cancer therapies include surgery, radiotherapy, chemotherapy, biological therapy, and combinations thereof. Where an anti-cancer therapy involves the use of a chemical or biological molecule or agent, the present compositions or kits-of-parts, such as particularly those where the Reoviridae virus is oncolytic, may further comprise said one or more chemical or biological molecules or agents.

The term “surgery” as used throughout this specification broadly denotes treatments comprising surgical removal of neoplastic tissue or cells from a subject. Cancer surgery may remove an entire tumor, debulk a tumor, or remove a tumor or a portion thereof causing pain or pressure. Cancer surgery includes inter alia conventional open surgery, laparoscopic surgery, cryosurgery, laser surgery, thermal ablation such as hyperthermic laser ablation or radiofrequency ablation, photodynamic therapy, and combinations thereof.

The term “radiotherapy” as used throughout this specification broadly denotes treatments comprising the exposure of neoplastic tissue to ionizing radiation, such as radiation from x-rays, gamma rays, neutrons, protons, or other sources. The source of the radiation may be an external apparatus (external-beam radiation therapy), or the radioactive material may be placed in the body near the neoplastic tissue (internal radiation therapy or brachytherapy), or radioactive material may be delivered systemically by injection, infusion or ingestion (systemic radioisotope therapy) and may concentrate in the neoplastic tissue spontaneously or by means of a targeting moiety, such as a cancer-targeting antibody.

The term “chemotherapy” as used herein is conceived broadly and generally encompasses treatments using chemical substances or compositions. Chemotherapeutic agents may typically display cytotoxic or cytostatic effects.

In certain embodiments, a chemotherapeutic agent may be an alkylating agent, a cytotoxic compound, an anti-metabolite, a plant alkaloid, a terpenoid, a topoisomerase inhibitor, or a combination thereof.

The term “alkylating agent” generally refers to an agent capable of alkylating nucleophilic functional groups under physiological conditions. Examples of alkylating agents include but are not limited to cyclophosphamide, carmustine, cisplatin, carboplatin, oxaliplatin, mechlorethamine, melphalan (hydrochloride), chlorambucil, ifosfamide, lomustine, mitomycin C, ThioTEPA, busulfan, and combinations thereof.

The term “cytotoxic compound” generally refers to an agent toxic to a cell. Examples of cytotoxic compounds include but are not limited to actinomycin (also known as dactinomycin); anthracyclines such as doxorubicin, daunorubicin, valrubicin, idarubicin, and epirubicin; bleomycin; plicamycin; mitoxantrone; mitomycin; and combinations thereof.

The term “anti-metabolite” generally refers to an agent capable to inhibit the use of a metabolite such as purines or pyrimidines. Anti-metabolites prevent purines and pyrimidines from becoming incorporated into DNA during the S phase of the cell cycle and thereby stop normal development and division. Examples of anti-metabolites include but are not limited to azathioprine, capecitabine, cytarabine, 5-fluorouracil, mercaptopurine, methotrexate, nelarabine, pemetrexed, and combinations thereof.

Plant alkaloids and terpenoids are derived from plants and block cell division by preventing microtubule function. Non-limiting examples include vinca alkaloids and taxanes, and combinations thereof. Examples of vinca alkaloids include but are not limited to vincristine, vinblastine, vinorelbine, vindesine, and combinations thereof. Examples of taxanes include but are not limited to paclitaxel, docetaxel, and combinations thereof.

The term “topoisomerase inhibitor” generally refers to enzymes that maintain the topology of DNA. Non-limiting examples include type I and type II topoisomerase inhibitors. Examples of type I topoisomerase inhibitors include but are not limited to camptothecins such as irinotecan, topotecan, and combinations thereof. Examples of type II topoisomerase inhibitors include but are not limited to amsacrine, doxorubicin, daunorubicin, etoposide, etoposide phosphate, mitoxantrone, teniposide, and combinations thereof.

In certain embodiments, a chemotherapeutic agent may be selected from the group consisting of cyclophosphamide, doxorubicin, idarubicin, mitoxantrone, oxaliplatin, bortezomib, digoxin, digitoxin, hypericin, shikonin, wogonin, sorafenib, everolimus, imatinib, geldanamycin, panobinostat, carmustine, cisplatin, carboplatin, mechlorethamine, melphalan (hydrochloride), chlorambucil, ifosfamide, busulfan, actinomycin, daunorubicin, valrubicin, epirubicin, bleomycin, plicamycin, mitoxantrone, mitomycin, azathioprine, mercaptopurine, fluorouracil, methotrexate, nelarabine, pemetrexed, vincristine, vinblastine, vinorelbine, vindesine, paclitaxel, docetaxel, irinotecan, topotecan, amsacrine, etoposide, etoposide phosphate, teniposide, anastrozole, exemestane, bosutinib, irinotecan, vandetanib, bicalutamide, lomustine, clofarabine, cabozantinib, cytarabine, cytoxan, decitabine, dexamethasone, hydroxyurea, decarbazine, leuprolide, epirubicin, asparaginase, estramustine, vismodegib, amifostine, flutamide, toremifene, fulvestrant, letrozole, degarelix, fludarabine, pralatrexate, floxuridine, gemcitabine, carmustine wafer, eribulin, altretamine, topotecan, axitinib, gefitinib, romidepsin, ixabepilone, ruxolitinib, cabazitaxel, carfilzomib, chlorambucil, sargramostim, cladribine, leuprolide, mitotane, procarbazine, megestrol, mesna, strontium-89 chloride, mitomycin, filgrastim, pegfilgrastim, sorafenib, nilutamide, pentostatin, tamoxifen, pegaspargase, denileukin diftitox, alitretinoin, carboplatin, prednisone, mercaptopurine, zoledronic acid, lenalidomide, octreotide, dasatinib, regorafenib, histrelin, sunitinib, omacetaxine, thioguanine, erlotinib, bexarotene, decarbazine, temozolomide, thiotepa, thalidomide, BCG, temsirolimus, bendamustine hydrochloride, triptorelin, arsenic trioxide, lapatinib, valrubicin intravesical, tretinoin, azacitidine, pazopanib, teniposide, leucovorin, crizotinib, capecitabine, enzalutamide, ziv-aflibercept, streptozocin, vemurafenib, goserelin, vorinostat, zoledronic acid, abiraterone, and combinations thereof.

The term “biological therapy” as used herein is conceived broadly and generally encompasses treatments using biological substances or compositions, such as biomolecules, or biological agents, such as viruses or cells. In certain embodiments, the biological substances or compositions may exert the pharmacological actions or effects underlying the therapeutic benefit. In certain other embodiments, the biological substances or compositions may be used to deliver or target chemotherapeutic agents or radioisotopes to the neoplastic tissues or cells, for example the biological substances or compositions may be conjugated with the chemotherapeutic agents or radioisotopes (by means of an example and without limitation, a conjugate of a cancer-targeting monoclonal antibody and a cytotoxic chemical compound).

In certain embodiments, a biomolecule may be a peptide, polypeptide, protein, nucleic acid, or a small molecule (such as primary metabolite, secondary metabolite, or natural product), or a combination thereof. Examples of suitable biomolecules include without limitation interleukins, cytokines, anti-cytokines, tumor necrosis factor (TNF), cytokine receptors, vaccines, interferons, enzymes, therapeutic antibodies, antibody fragments, antibody-like protein scaffolds, or combinations thereof.

Examples of suitable biomolecules include but are not limited to aldesleukine, alemtuzumab, atezolizumab, bevacizumab, blinatumomab, brentuximab vedotine, catumaxomab, cetuximab, daratumumab, denileukin diftitox, denosumab, dinutuximab, elotuzumab, gemtuzumab ozogamicin, ⁹⁰Y-ibritumomab tiuxetan, idarucizumab, interferon A, ipilimumab, necitumumab, nivolumab, obinutuzumab, ofatumumab, olaratumab, panitumumab, pembrolizumab, ramucirumab, rituximab, tasonermin, ¹³¹I-tositumomab, trastuzumab, Ado-trastuzumab emtansine, fam-trastuzumab deruxtecan-nxki, and combinations thereof.

Examples of suitable oncolytic viruses include but are not limited to talimogene laherparepvec (oncolytic herpes simplex virus).

Further categories of anti-cancer therapy include inter alia hormone therapy (endocrine therapy), immunotherapy, and stem cell therapy, which are commonly considered as subsumed within biological therapies.

Hormone therapy or endocrine therapy encompasses treatments in which hormones or anti-hormone drugs are administered for the treatment of hormone-dependent or hormone-sensitive cancers, such as inter alia hormone-dependent or hormone-sensitive breast cancer, prostate cancer, ovarian cancer, testicular cancer, endometrial cancer, or kidney cancer.

Examples of suitable hormone therapies include but are not limited to tamoxifen; aromatase inhibitors, such as atanastrozole, exemestane, letrozole, and combinations thereof; luteinizing hormone blockers such as goserelin, leuprorelin, triptorelin, and combinations thereof; anti-androgens, such as bicalutamide, cyproterone acetate, flutamide, and combinations thereof; gonadotrophin releasing hormone blockers, such as degarelix; progesterone treatments, such as medroxyprogesterone acetate, megestrol, and combinations thereof; and combinations thereof.

The term “immunotherapy” broadly encompasses any treatment that modulates a subject's immune system. In particular, the term comprises any treatment that modulates an immune response, such as a humoral immune response, a cell-mediated immune response, or both. An immune response may typically involve a response by a cell of the immune system, such as a B cell, cytotoxic T cell (CTL), T helper (Th) cell, regulatory T (Treg) cell, antigen-presenting cell (APC), dendritic cell, monocyte, macrophage, natural killer T (NKT) cell, natural killer (NK) cell, basophil, eosinophil, or neutrophil, to a stimulus. In the context of anti-cancer treatments, immunotherapy may preferably elicit, induce or enhance an immune response, such as in particular an immune response specifically against tumor tissues or cells, such as to achieve tumor cell death. Immunotherapy may modulate, such increase or enhance, the abundance, function, and/or activity of any component of the immune system, such as any immune cell, such as without limitation T cells (e.g., CTLs or Th cells), dendritic cells, and/or NK cells.

Immunotherapy comprises cell-based immunotherapy in which immune cells, such as T cells and/or dendritic cells, are transferred into the patient. The term also comprises an administration of substances or compositions, such as chemical compounds and/or biomolecules (e.g., antibodies, antigens, interleukins, cytokines, or combinations thereof), that modulate a subject's immune system.

Examples of cancer immunotherapy include without limitation treatments employing monoclonal antibodies, for example Fc-engineered monoclonal antibodies against proteins expressed by tumor cells, immune checkpoint inhibitors, prophylactic or therapeutic cancer vaccines, adoptive cell therapy, and combinations thereof.

Immune checkpoints are inhibitory pathways that slow down or stop immune reactions and prevent excessive tissue damage from uncontrolled activity of immune cells. Inhibition of immune checkpoint targets can stimulate immune responses by immune cells, such as CTLs, against tumor cells.

Examples of immune checkpoint targets for inhibition include without limitation PD-1 (examples of PD-1 inhibitors include without limitation pembrolizumab, nivolumab, and combinations thereof), CTLA-4 (examples of CTLA-4 inhibitors include without limitation ipilimumab, tremelimumab, and combinations thereof), PD-L1 (examples of PD-L1 inhibitors include without limitation atezolizumab), LAG3, B7-H3 (CD276), B7-H4, TIM-3, BTLA, A2aR, killer cell immunoglobulin-like receptors (KIRs), IDO, and combinations thereof.

In certain embodiments, the Reoviridae virus is an attenuated live virus. The term “attenuated” is well-known in the field of vaccination and when used in combination with a virus, denotes a virus variant or mutant which exhibits a substantially lower degree of virulence compared to a wild-type virus in an intended recipient, such as a human or a non-human animal, while retaining the ability to stimulate an immune response similar to the wild type virus, preferably a virus variant or mutant exhibiting reduced propagation in the host (i.e., in vivo), e.g., due to slower growth rate and/or a reduced level of replication compared to a wild-type virus. Propagation of an attenuated virus in the host (i.e., in vivo) may be at least about 10 fold, e.g., at least about 25 fold, or at least about 50 fold, or at least about 75 fold, preferably at least about 100 fold, less than that of a wild-type virus. Typically, such attenuated virus will not induce symptoms of viral infection or will induce only mild symptoms upon infecting, preferably through vaccination, a subject, but severe symptoms of viral infection do not typically occur in the infected, preferably vaccinated, subject. Suitable methods for measuring the propagation or virulence of a virus have been described elsewhere in this specification. Standard methods of attenuating viruses are generally known and may include passage of the virus through a foreign host, such as in vitro cultured cells of a foreign host, embryonated eggs, or live non-human animals, or random or directed mutagenesis of the wild-type virus.

The composition or kit-of-parts comprising the Reoviridae virus, such as the attenuated live Reoviridae virus, and the sialic acid and/or the molecule comprising at least one sialic acid moiety as taught herein, is useful for therapy, and particularly useful in immunisation against the Reoviridae virus. Hence, an aspect provides the composition or kit-of-parts comprising the Reoviridae virus, such as the attenuated live Reoviridae virus, and the sialic acid and/or the molecule comprising at least one sialic acid moiety as taught herein, for use in therapy.

A further aspect provides the composition or kit-of-parts comprising the Reoviridae virus, such as particularly the attenuated live Reoviridae virus, and the sialic acid and/or the molecule comprising at least one sialic acid moiety as taught herein, for use in a method of immunisation against the Reoviridae virus. A related aspect provides a method of immunisation against a Reoviridae virus in a subject, comprising administering to the subject a therapeutically or prophylactically effective amount of the Reoviridae virus, such as particularly the attenuated live Reoviridae virus, and the sialic acid and/or the molecule comprising at least one sialic acid moiety as taught herein.

Hence, such aspects and embodiments provide the compositions and kits-of-parts as vaccines against the Reoviridae virus. The term “vaccine” generally refers to a therapeutic or prophylactic pharmaceutical composition for in vivo administration to a subject, comprising a component to which a vaccinated subject is induced to raise an immune response, preferably a protective immune response.

Optionally, the vaccine may further comprise one or more adjuvants for enhancing the immune response. Suitable adjuvants include, for example, but without limitation, saponin, mineral gels such as aluminium hydroxide, surface active substances such as lysolecithin, pluronic polyols, polyanions, peptides, oil or hydrocarbon emulsions, keyhole limpet hemocyanin (KLH), monophosphoryl lipid A (MPL), Corynebacterium parvum, oligodeoxynucleotides containing unmethylated CpG motif, and QS-21. An example is Freund's adjuvant.

Optionally, the vaccine may further comprise one or more immunostimulatory molecules. Non-limiting examples of such molecules include various cytokines, lymphokines and chemokines. By means of example, non-limiting examples of molecules with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc.

Illustrative vaccines against reovirus infection are commercially available, and constitute embodiments useful in practicing the present invention, such as for example Nobilis® REO 1133 from MSD Animal Health for chickens, or the rotavirus vaccines Rotarix (GlaxoSmithKline) or RotaTeq® (Merck Vaccines).

The compositions and kits-of-parts as taught herein may be formulated as pharmaceutical compositions or kits of parts with a pharmaceutically acceptable excipient, i.e., one or more pharmaceutically acceptable carrier substances and/or additives, e.g., buffers, carriers, excipients, stabilisers, etc. The term “pharmaceutically acceptable” as used herein is consistent with the art and means compatible with the other ingredients of the pharmaceutical composition and not deleterious to the recipient thereof. Accordingly, an aspect provides a pharmaceutical composition comprising the Reoviridae virus and the sialic acid and/or the molecule comprising at least one sialic acid moiety as taught herein. A further aspect provides a pharmaceutical kit-of-parts comprising the Reoviridae virus and the sialic acid and/or the molecule comprising at least one sialic acid moiety as taught herein. In certain embodiment, the pharmaceutical composition or kit-of-parts may be a vaccine as described elsewhere in this specification.

The terms “pharmaceutical composition” and “pharmaceutical formulation” may be used interchangeably. The pharmaceutical formulations or kits-of-parts as taught herein may comprise in addition to the herein particularly specified components one or more pharmaceutically acceptable excipients. Suitable pharmaceutical excipients depend on the dosage form and identities of the active ingredients and can be selected by the skilled person (e.g., by reference to the Handbook of Pharmaceutical Excipients 7th Edition 2012, eds. Rowe et al.). As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. Acceptable diluents, carriers and excipients typically do not adversely affect a recipient's homeostasis (e.g., electrolyte balance). The use of such media and agents for pharmaceutical active substances is well known in the art. Such materials should be non-toxic and should not interfere with the activity of the active pharmaceutical ingredient. Acceptable carriers may include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride.

The precise nature of the carrier or other material will depend on the route of administration. For example, the pharmaceutical composition may be in the form of a parenterally acceptable aqueous solution, which is pyrogen-free and has suitable pH, isotonicity and stability.

The pharmaceutical formulations may comprise pharmaceutically acceptable auxiliary substances as required to approximate physiological conditions, such as pH adjusting and buffering agents, preservatives, complexing agents, tonicity adjusting agents, wetting agents and the like, for example, sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, triethanolamine oleate, etc. Preferably, the pH value of the pharmaceutical formulation is in the physiological pH range, such as particularly the pH of the formulation is between about 5 and about 9.5, more preferably between about 6 and about 8.5, even more preferably between about 7 and about 7.5. The preparation of such pharmaceutical formulations is within the ordinary skill of a person skilled in the art.

Administration of the pharmaceutical composition can be systemic or local. Pharmaceutical compositions can be formulated such that they are suitable for parenteral and/or non-parenteral administration. Specific administration modalities include subcutaneous, intravenous, intramuscular, intraperitoneal, transdermal, intrathecal, oral, rectal, buccal, topical, nasal, ophthalmic, intra-articular, intra-arterial, sub-arachnoid, bronchial, lymphatic, vaginal, and intra-uterine administration.

In certain preferred embodiments, the administration may be intravenous (IV), such as IV infusion or IV injection.

In certain preferred embodiments, the administration may be subcutaneous, such as subcutaneous injection.

In certain preferred embodiments, the administration may be or intraperitoneal (IP), such as IP injection.

Administration can be by periodic injections of a bolus of the pharmaceutical composition or can be uninterrupted or continuous by intravenous, subcutaneous or intraperitoneal administration from a reservoir which is external (e.g., an IV bag) or internal (e.g., a bioerodable implant, a bioartificial organ, or a colony of implanted host cells). Administration of a pharmaceutical composition can be achieved using suitable delivery means such as: a pump, microencapsulation, continuous release polymer implants, macroencapsulation, injection, either subcutaneously, intravenously, intra-arterially, intramuscularly, or to other suitable site, or oral administration, in capsule, liquid, tablet, pill, or prolonged release formulation.

Examples of parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aerosolizer, electroporation, and transdermal patch.

Formulations suitable for parenteral administration conveniently contain a sterile aqueous preparation of the active pharmaceutical ingredient, which preferably is isotonic with the blood of the recipient (e.g., physiological saline solution). Formulations can be presented in unit-dose or multi-dose form.

Formulations suitable for oral administration can be presented as discrete units such as capsules, cachets, tablets, or lozenges, each containing a predetermined amount of the active pharmaceutical ingredient, or a suspension in an aqueous liquor or a non-aqueous liquid, such as a syrup, an elixir, an emulsion, or a draught.

Formulations suitable for topical administration can be presented as, e.g., a cream, a spray, a foam, a gel, an ointment, a salve, or a dry rub. A dry rub can be rehydrated at the site of administration. Such formulations can also be infused directly into (e.g., soaked into and dried) a bandage, gauze, or patch, which can then be applied topically. Such formulations can also be maintained in a semi-liquid, gelled, or fully-liquid state in a bandage, gauze, or patch for topical administration.

In certain embodiments, the active pharmaceutical ingredient may be lyophilised. Any of the pharmaceutical compositions described herein can be included in a container, pack, or dispenser together with instructions for administration. In some embodiments, the composition is packaged as a single use vial, such as a single use syringe.

In certain embodiments, the composition or any of the components of the kit-of-parts may be cryopreserved or lyophilised.

One skilled in this art will recognise that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The term “sialic acid” is well-known in the art and by means of further guidance constitutes a generic term for the N- and/or O-substituted derivatives of neuraminic acid (Neu). Neu is a nine-carbon monosaccharide ((4S,5R,6R,7S,8R)-5-amino-4,6,7,8,9-pentahydroxy-2-oxononanoic acid), depicted by the following formula:

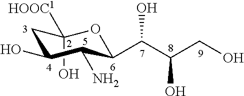

In certain embodiments, the sialic acid is N-substituted neuraminic acid, or the at least one sialic acid moiety is an N-substituted neuraminic acid moiety. In certain embodiments, the sialic acid is O-substituted neuraminic acid, or the at least one sialic acid moiety is an O-substituted neuraminic acid moiety. In certain embodiments, the sialic acid is N-substituted and O-substituted neuraminic acid, or the at least one sialic acid moiety is an N-substituted and O-substituted neuraminic acid moiety. In certain embodiments, the sialic acid is O-substituted neuraminic acid or N- and 0-substituted neuraminic acid, or the at least one sialic acid moiety is an O-substituted neuraminic acid moiety or an N- and O-substituted neuraminic acid moiety, wherein two or more of the hydroxyl groups of the neuraminic acid or the neuraminic acid moiety are substituted, such as two, three, four or five of the hydroxyl groups. In particular, hydroxyl groups are present at C2, C4, C7, C8, and C9.

The nature of the substituents may vary. Typically, the amino group at C5 of Neu may be substituted by an acetyl or a glycolyl group, but other substituents have been described, such as hydroxyl, acetimidoyl, acetyl-O-glycolyl, methyl-O-glycolyl, or N-glycolylneuraminic acid-2-O-5-glycolyl.

In certain preferred embodiments, the neuraminic acid or the neuraminic acid moiety is N-substituted with an acetyl group or a glycolyl group, preferably with an acetyl group, or in other words, the sialic acid or the at least one sialic acid moiety comprises an N-acetyl or N-glycolyl group, preferably N-acetyl group, at C5.

Hence, in certain embodiments, the sialic acid is N-acetylneuraminic acid (Neu5Ac) or N-glycolylneuraminic acid (Neu5Gc). In preferred embodiments, the sialic acid is Neu5Ac. In even more preferred embodiments, the composition or kit-of-parts comprises Neu5Ac. In certain embodiments, the at least one sialic acid moiety is a Neu5Ac moiety or a Neu5Gc moiety. In preferred embodiments, the at least one sialic acid moiety is a Neu5Ac moiety. In certain embodiments, the composition or kit-of-parts comprises a molecule comprising at least one Neu5Ac moiety.

In certain embodiments, the neuraminic acid or the neuraminic acid moiety is N-substituted, but is not O-substituted.

In certain embodiments, the hydrogen in one or more hydroxyl groups of the neuraminic acid or the N-substituted neuraminic acid, or of the neuraminic acid moiety or the N-substituted neuraminic acid moiety, is substituted. Typical O-linked substituents in sialic acid may, each independently, be selected from the group comprising or consisting of acetyl, methyl, lactyl, sulphate, phosphate, D-galactosyl (Gal), D-fucosyl (Fuc), D-glucosyl (Glc), and sialyl.

More typically, O-linked substituents at C4 (if the —OH group at C4 is substituted) may be selected from the group comprising or consisting of acetyl, Fuc, and Gal; O-linked substituent at C7 (if the —OH group at C7 is substituted) may be acetyl; O-linked substituents at C8 (if the —OH group at C8 is substituted) may be selected from the group comprising or consisting of acetyl, methyl, sulphate, Sia, and Glc; and/or O-linked substituents at C9 (if the —OH group at C9 is substituted) may be selected from the group comprising or consisting of acetyl, lactyl, phosphate, sulphate, and Sia. In certain embodiments, anhydro linkages (C—O—C) may be formed between C4 and C8 and/or between C2 and C7.

In certain embodiments, the sialic acid is N-acetylneuraminic acid (Neu5Ac) or N-glycolylneuraminic acid (Neu5Gc), optionally wherein one or more hydroxyl groups of said Neu5Ac or Neu5Gc are each independently substituted, such as with acetyl, methyl, lactyl, sulphate or phosphate; or wherein the at least one sialic acid moiety is a Neu5Ac or Neu5Gc moiety, optionally wherein one or more hydroxyl groups of said Neu5Ac or Neu5Gc moiety are each independently substituted, such as with acetyl, methyl, lactyl, sulphate or phosphate.

The sialic acid or the at least one sialic acid moiety may be in a free acid form (—COOH, or dissociated to —COO⁻ and H⁺), or may be in the form of salts, in particular pharmaceutically acceptable salts, e.g., may be converted into metal or amine addition salt forms by treatment with appropriate organic and inorganic bases. Appropriate base addition salt forms comprise, for example, the ammonium salts, the alkali and earth alkaline metal salts, e.g. the lithium, sodium, potassium, magnesium, calcium salts and the like, aluminum salts, zinc salts, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The nature or structure of the molecule comprising the at least one sialic acid moiety is not limited, insofar the molecule allows for a contact or interaction between the at least one sialic acid moiety and the Reoviridae virus (more particularly with an outer capsid protein of the virus, even more particularly with sigma-1 protein of an Orthoreovirus, such as avian or mammalian reovirus) in accordance with the principles of the invention. By means of further guidance and without limitation, the molecule may be such that the at least one sialic acid moiety is at least partly or fully exposed to the environment or solvent, and that the remainder of the molecule does not sterically or otherwise hinder the contact or interaction of the sialic acid moiety with the virus. Illustrative but non-limiting examples of molecules which may comprise the at least one sialic acid moiety include oligosaccharides, polysaccharides, peptides, polypeptides, proteins, protein domains, protein complexes, dextran, polyethylene glycol, small molecules, or combinations thereof (e.g., an oligosaccharide or polysaccharide bound to a peptide, polypeptide, or protein). Such molecules may be preferably pharmaceutically acceptable.

The at least one sialic acid moiety may be covalently bound to the remainder of the molecule, and may more typically be bound via one of its C atoms containing a hydroxyl group, even more typically via its C2 atom. The linkage may involve a C—O—C bond between the at least one sialic acid moiety and the remainder of the molecule.

In certain embodiments, the molecule comprises or consists of an oligosaccharide comprising the at least one sialic acid moiety. In certain embodiments, the molecule comprises or consists of a polysaccharide comprising the at least one sialic acid moiety.

The term “oligosaccharide” broadly refers to compounds in which 2 to 20 monosaccharide units are joined by glycosidic linkages. According to the number of units, they are called disaccharides, trisaccharides, tetrasaccharides, pentasaccharides etc. For example, an oligosaccharide may comprise or consist of a sialic acid moiety and one or more than one further monosaccharide units, such as 1, 2, 3, 4, 5, 6, 7, 8 or 9 further monosaccharide units. For example, an oligosaccharide may comprise or consist of two sialic acid moieties. For example, an oligosaccharide may comprise or consist of two sialic acid moieties, and one or more than one further monosaccharide units. For example, an oligosaccharide may comprise or consist of three or more sialic acid moieties. For example, an oligosaccharide may comprise or consist of three or more sialic acid moieties, and one or more than one further monosaccharide units. The term “polysaccharide” broadly refers to a polymer or macromolecule consisting of monosaccharide units, such as more than 20 monosaccharide units, joined together by glycosidic bonds. Oligosaccharides or polysaccharides may be linear or branched.

Illustrative, but non-limiting examples of monosaccharide units which may be comprised by oligosaccharides or polysaccharides as intended herein include D-Glucose, D-Galactose, L-Galactose, D-Mannose, D-Allose, L-Altrose, D-Gulose, L-Idose, D-Talose, D-Ribose, D-Arabinose, L-Arabinose, D-Xylose, D-Lyxose, D-Erythrose, D-Threose, L-glycero-D-manno-Heptose, D-glycero-D-manno-Heptose, 6-Deoxy-L-altrose, 6-Deoxy-D-talose, D-Fucose, L-Fucose, D-Rhamnose, L-Rhamnose, D-Quinovose, 2-Deoxyglucose, 2-Deoxyribose, Olivose, Tyvelose, Ascarylose, Abequose, Paratose, Digitoxose, Colitose, D-Glucosamine, D-Galactosamine, D-Mannosamine, D-Allosamine, L-Altrosamine, D-Gulosamine, L-Idosamine, D-Talosamine, N-Acetyl-D-glucosamine, N-Acetyl-D-galactosamine, N-Acetyl-D-mannosamine, N-Acetyl-D-allosamine, N-Acetyl-L-altrosamine, N-Acetyl-D-gulosamine, N-Acetyl-L-idosamine, N-Acetyl-D-talosamine, N-Acetyl-D-fucosamine, N-Acetyl-L-fucosamine, N-Acetyl-L-rhamnosamine, N-Acetyl-D-quinovosamine, D-Glucuronic acid, D-Galacturonic acid, D-Mannuronic acid, D-Alluronic acid, L-Altruronic acid, D-Guluronic acid, L-Guluronic acid, L-Iduronic acid, D-Taluronic acid, and combinations thereof. The term may also encompass sugar alcohols, such as Erythritol, Arabinitol, Xylitol, Ribitol, Glucitol, Galactitol, and/or Mannitol. The term may also encompass ketoses, such as D-Psicose, D-Fructose, L-Sorbose, D-Tagatose, D-Xylulose, and/or D-Sedoheptulose. Any such monosaccharide units, particularly one or more hydroxyl groups thereof, may be substituted by one or more other functional group, such as without limitation acetyl, methyl, lactyl, sulphate, and/or phosphate.

In certain embodiments, the molecule comprises or consists of an oligosaccharide or a polysaccharide, wherein the at least one sialic acid moiety is bound to the underlying monosaccharide unit via the C2 carbon of the sialic acid moiety by a glycosidic bond. In certain preferred embodiments, the molecule comprises or consists of an oligosaccharide or a polysaccharide, wherein the at least one sialic acid moiety is bound to the underlying monosaccharide unit via the C2 carbon of the sialic acid moiety by an alpha glycosidic bond (i.e., α-linked sialic acid moiety). In certain embodiments, the underlying monosaccharide unity is each independently Galactose, N-Acetylgalactosamine, N-Acetylglucosamine, or Sialic acid. In certain embodiments, the sialic acid moiety is, each independently, bound via its C2 carbon by an α-glycosidic bond to C3, C4 or C6 of Galactose, C6 of N-Acetylgalactosamine, C4 or C6 of N-Acetylgalactosamine, or C8 or C9 of sialic acid.

In certain embodiments, the molecule comprises or consists of an oligosaccharide or a polysaccharide comprising the at least one sialic acid moiety as a terminal moiety. Hence, such oligosaccharide or polysaccharide comprises at least one terminal sialic acid moiety, more particularly at least one α-linked terminal sialic acid moiety, more particularly at least one terminal sialic acid moiety bound to the underlying monosaccharide unit via the C2 carbon of the sialic acid moiety by an α-glycosidic bond. Such oligosaccharide or polysaccharide may comprise one or more (e.g., in branched structures) sialic acid moieties which are terminal, and may optionally also comprise one or more sialic acid moieties which are not terminal. A terminal sialic acid moiety will thus form a glycosidic bond (e.g., α-glycosidic bond via its C2) with an underlying monosaccharide unit in the oligosaccharide or polysaccharide, but will not be interposed between the underlying monosaccharide unit and another, ensuing monosaccharide unit. For example, C7, C8 and C9 of the terminal sialic acid moiety will not be involved in a glycosidic bond.

In certain embodiments, the molecule comprising at least one sialic acid moiety is sialyl-lacto-N-tetraose (LSTa). In even more preferred embodiments, the composition or kit-of-parts comprises LSTa.

In certain embodiments, the molecule comprising at least one sialic acid moiety is α-2,3-sialyllactose, α-2,6-sialyllactose, or α-2,8-disiallylactose. In certain embodiments, the composition or kit-of-parts comprises α-2,3-sialyllactose, α-2,6-sialyllactose, or α-2,8-disiallylactose.

In certain embodiments, the sialic acid or the molecule comprising at least one sialic acid moiety may be linked to a macromolecular structure, such as a polymer carrier or bead or support, such as an agarose bead, a latex bead, a cellulose bead, a magnetic bead, a silica bead, a polyacrylamide bead, or a glass bead, optionally via a linker.

Sialic acids and molecules comprising (terminal) sialic acid, such as oligosaccharides and polysaccharides, are found widely distributed in animal tissues, as well as in fungi and yeasts (e.g., in glycans of glycoproteins and gangliosides), and can be isolated therefrom as known in the art. As an example, N-acetyl neuraminic acid can be commercially purchased (e.g., Sigma-Aldrich cat. no. A0812).

In the context of the compositions or kits-of-parts described herein, any quantity of the Reoviridae virus suitable for achieving the desired effect, such as an oncolytic or immunising effect, is envisaged. By means of an example and without limitation, the amount of the Reoviridae virus in a single dose may be between 10² and 10¹⁰ CCID₅₀ (50% cell culture infectious dose), such as between 10³ and 10⁹ CCID₅₀, such as between 10⁴ and 10⁸ CCID₅₀, such as between 10⁵ and 10⁷ CCID₅₀, such as at least about 10⁶ CCID₅₀.

Moreover, any quantity of the sialic acid and/or the molecule comprising at least one sialic acid moiety suitable for enhancing the binding of the Reoviridae virus to the host cells and/or increasing the infectivity of the virus in envisaged. By means of an example and without limitation, the virus may be contacted with a concentration of the sialic acid, such as NeuAc, ranging from 1 μM to 1M, such as from 10 μM to 100 mM, such as from 100 μM to 10 mM, such as about 1 mM, about 2 mM, about 3 mM, about 4 mM, about 5 mM, about 6 mM, about 7 mM, about 8 mM, about 9 mM, or about 10 mM. By means of an example and without limitation, the virus may be contacted with a concentration of the molecule comprising at least one sialic acid moiety, such as LSTa, ranging from 1 μM to 1M, such as from 10 μM to 100 mM, such as from 100 μM to 10 mM, such as about 1 mM, about 2 mM, about 3 mM, about 4 mM, about 5 mM, about 6 mM, about 7 mM, about 8 mM, about 9 mM, or about 10 mM. By means of an example and without limitation, the virus may be contacted with a concentration of the molecule comprising at least one sialic acid moiety, such that the resulting concentration of α-linked terminal sialic acid moieties ranges from 1 μM to 1M, such as from 10 μM to 100 mM, such as from 100 μM to 10 mM, such as about 1 mM, about 2 mM, about 3 mM, about 4 mM, about 5 mM, about 6 mM, about 7 mM, about 8 mM, about 9 mM, or about 10 mM.

The present application also provides aspects and embodiments as set forth in the following Statements:

Statement 1. A composition or a kit-of-parts comprising i) a virus which is a member of the Reoviridae family and ii) sialic acid and/or a molecule comprising at least one sialic acid moiety.

Statement 2. The composition or kit-of-parts according to Statement 1, wherein the Reoviridae virus displays host tropism for at least one vertebrate species.

Statement 3. The composition or kit-of-parts according to Statement 1 or 2, wherein the Reoviridae virus displays host tropism for at least one mammalian species.

Statement 4. The composition or kit-of-parts according to any one of Statements 1 to 3, wherein the Reoviridae virus displays host tropism for humans.

Statement 5. The composition or kit-of-parts according to any one of Statements 1 to 4, wherein the Reoviridae virus is an Orthoreovirus, Orbivirus, or Rotavirus.

Statement 6. The composition or kit-of-parts according to any one of Statements 1 to 5, wherein the Reoviridae virus comprises an outer capsid and an inner core.

Statement 7. The composition or kit-of-parts according to any one of Statements 1 to 6, wherein the Reoviridae virus comprises an outer capsid protein capable of binding to a host cell surface receptor, wherein the sialic acid or the molecule comprising the at least one sialic acid moiety causes said outer capsid protein to adopt a more elongated or extended conformation on the Reoviridae virus compared to the conformation in the absence of the sialic acid or the molecule comprising the at least one sialic acid moiety.

Statement 8. The composition or kit-of-parts according to Statements 7, wherein the outer capsid protein is sigma-1 protein.

Statement 9. The composition or kit-of-parts according to any one of Statements 1 to 8, wherein the sialic acid is N-substituted neuraminic acid, or wherein the at least one sialic acid moiety is an N-substituted neuraminic acid moiety, optionally wherein said N-substituted neuraminic acid or said N-substituted neuraminic acid moiety is further O-substituted.

Statement 10. The composition or kit-of-parts according to any one of Statements 1 to 9, wherein the sialic acid is N-acetylneuraminic acid (Neu5Ac) or N-glycolylneuraminic acid (Neu5Gc), optionally wherein one or more hydroxyl groups of said Neu5Ac or Neu5Gc are each independently substituted, such as with acetyl, methyl, lactyl, sulphate or phosphate; or wherein the at least one sialic acid moiety is a Neu5Ac or Neu5Gc moiety, optionally wherein one or more hydroxyl groups of said Neu5Ac or Neu5Gc moiety are each independently substituted, such as with acetyl, methyl, lactyl, sulphate or phosphate.

Statement 11. The composition or kit-of-parts according to any one of Statements 1 to 10, wherein the sialic acid is Neu5Ac or wherein the at least one sialic acid moiety is a Neu5Ac moiety, preferably wherein the composition or kit-of-parts comprises Neu5Ac.

Statement 12. The composition or kit-of-parts according to any one of Statements 1 to 11, wherein the molecule comprises or consists of an oligosaccharide or a polysaccharide comprising the at least one sialic acid moiety as a terminal moiety.

Statement 13. The composition or kit-of-parts according to any one of Statements 1 to 12, wherein the Reoviridae virus is an oncolytic virus.

Statement 14. The composition or kit-of-parts according to Statement 13, wherein the oncolytic Reoviridae virus is linked to a binding agent, such as an antibody, capable of specifically binding to neoplastic cells, optionally wherein the sialic acid and/or the molecule comprising the at least one sialic acid moiety is also linked to said binding agent.

Statement 15. The composition or kit-of-parts according to any one of Statements 1 to 12, wherein the Reoviridae virus is an attenuated live virus.

Statement 16. The composition or kit-of-parts according to any one of Statements 1 to 15 for use in therapy.

Statement 17. The composition or kit-of-parts according to Statement 13 or 14 for use in a method of treating a neoplastic disease.

Statement 18. The composition or kit-of-parts according to Statement 15 for use in a method of immunisation against the Reoviridae virus.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1—Materials and Methods Used in Examples 2-5

Generation of Reovirus Stocks

T3SA+ and T3SA− reovirus stocks have been previously described (Frierson et al. 2012, supra). T3SA+ corresponds to wild-type strain type 3 Dearing (T3D), and T3SA− corresponds to a T3D derivative carrying a point mutation in the sigma-1 (σ1) protein, namely R202W (i.e., T3D-σ1R202W), whereas other point mutations located in the same region of the σ1 protein, and more particularly mutations at N198, R202, or P204 can also abolish binding of T3 al with sialic acid (see Reiter et al. Crystal Structure of Reovirus Attachment Protein σ1 in Complex with Sialylated Oligosaccharides PLoS Pathog 2011, vol. 7(8), e1002166). T3SA+ and T3SA− reovirus stocks were prepared by plaque purification and passaging the viruses 3-4 times in L929 cells (ATCC, #CCL-1). Purified virions were prepared from infected L929 cell lysates by cesium chloride gradient centrifugation as described (Furlong et al. Sigma 1 protein of mammalian reoviruses extends from the surfaces of viral particles. J. Virol. 1988, vol. 62, 246-256). Briefly, infected cells were lysed by sonication, and virions were extracted from lysates using vertrel-XF (Furlong et al., supra; Mendez et al. A comparative analysis of freon substitutes in the purification of reovirus and calicivirus. J. Virol. Methods 2000, vol. 90, 59-67). The extracted virions were layered onto 1.2 to 1.4 g/cm³ caesium chloride step gradients and centrifuged at 25000 rpm at 4° C. for 18 h. The band corresponding to the density of reovirus particles (˜1.36 g/cm³) was collected and exhaustively dialyzed against virion-storage buffer (150 mM NaCl, 15 mM MgCl2, and 10 mM Tris [pH 7.4]). Particle concentration was determined from optical density at 260 nm (1 OD₂₆₀=2.1×10¹² particles/mL) (Smith et al. Polypeptide components of virions, top component and cores of reovirus type 3. Virology 1969, vol. 39, 791-810). Infectious subvirion particles (ISVPs) were generated by incubation of virions (2×10¹² particles/mL) with 2 mg/mL α-chymotrypsin (Sigma-Aldrich) at 37° C. for 60 min (Baer & Dermody. Mutations in reovirus outer-capsid protein sigma3 selected during persistent infections of L cells confer resistance to protease inhibitor E64. J. Virol. 1997, vol. 71, 4921-4928). The reaction was quenched by incubation on ice and addition of phenylmethylsulfonyl fluoride (Sigma-Aldrich) to a concentration of 2 mM. Reovirus particles, diluted into fresh 50 mM sodium bicarbonate (pH 8.5; 6×10¹² particles/mL) were labeled by incubation with 20 μM succinimidyl ester of Alexa Flour 488 (Invitrogen) at room temperature for 90 min in the dark to generate fluoresceinated virions. Unreacted dye was removed by dialysis against PBS at 4° C. overnight (Mainou & Dermody. Transport to late endosomes is required for efficient reovirus infection. J. Virol. 2012, vol. 86). Fluoresceinated ISVPs were prepared by α-chymotrypsin treatment of fluoresceinated virions. Viral titers were determined by plaque assay using L929 cells (Virgin et al. Antibody protects against lethal infection with the neurally spreading reovirus type 3 (Dearing). J. Virol. 1988, vol. 62, 4594-4604).

Cell Lines

Engineering and characterization of cell lines expressing JAM-A. Monolayers of CHO (ATCC, #CCL-61) and Lec2 (ATCC, #CRL-1736) cells were transduced with lentiviruses encoding a puromycin resistance gene and human JAM-A or a puromycin resistance gene alone. Transduced cells were selected for puromycin resistance by passaging twice in medium containing 20 μg ml⁻¹ puromycin. The concentration of puromycin used was the minimal concentration that yielded complete death of non-transduced CHO and Lec2 cells. Following selection for puromycin resistance, cells were further selected for cell surface expression of JAM-A using fluorescence-activated cell sorting (FACS). Cell-surface expression of JAM-A was detected using the monoclonal antibody, J10.4 (Liu et al. Human junction adhesion molecule regulates tight junction resealing in epithelia. J. Cell Sci. 2000, vol. 113, 2363-2374), and a fraction of cells with high JAM-A expression was collected and propagated using puromycin selection. In the ensuing Examples, cells transduced and selected for puromycin resistance alone will be referred to as CHO and Lec2 and those selected for puromycin resistance and JAM-A expression will be referred to as CHO-JAM-A and Lec2-JAM-A, respectively.

Culture of CHO cell lines. CHO cells (CHO, CHO-JAM-A) were grown in Ham's F12 medium (Sigma-Aldrich) supplemented to contain 10% fetal bovine serum (FBS), penicillin (100 U ml⁻¹), and streptomycin (100 μg ml⁻¹) (Invitrogen) at 37° C. in a humidified atmosphere with 5% CO₂. During alternate passages, 20 μg ml⁻¹ puromycin was added to the medium.

Culture of Lec2 cell lines. Lec2 cells (Lec2, Lec2-JAM-A) were grown in Mem a, nucleosides medium (Gibco) supplemented to contain 10% FBS, penicillin (100 U ml⁻¹), and streptomycin (100 μg ml⁻¹) at 37° C. in a humidified atmosphere with 5% CO₂. During alternate passages, 20 μg ml⁻¹ puromycin was included in the medium.

Transduction of CHO and Lec2 cells. The four cell types used in the ensuing Examples were transduced to express nuclear GFP as well as cytoplasmic mCherry using H2B-eGFP- and actin-mCherry-expressing lentiviruses, respectively, as described (Salmon & Trono. Production and titration of lentiviral vectors. Current protocols in human genetics 2007, vol. 54, 12.10.11-12.10.24). Cells expressing both GFP and mCherry were selected by FACS and propagated using the culture conditions described above. In addition, Lec2-JAM-A expressing mCherry only, used for single-particle tracking experiments, also were selected and propagated as described previously.

FACS of transduced cells. Cells transduced with GFP and actin-mCherry transgenes were trypsinized and collected into PBS with 2 mM EDTA and 1% FBS. The cells were sorted using a BD FACSARIA III cell sorter, with a nozzle of 85 μm, sheath pressure of 45 psi, drop frequency of 47 kHz, and sort precision of 0-32-0. GFP was excited with a 488 nm laser and emission filtered with a 530/30 band pass filter and 505 long-pass mirror. mCherry was excited with a 561 nm (yellow-green) laser and emission-filtered with a 610/20 band-pass filter. Cells expressing both GFP and mCherry were collected and propagated using the culture conditions described above.

Functionalization of AFM tips

NHS-PEG₂₇-acetal linkers were used to functionalize AFM tips as described (Gruber. Crosslinkers and Protocols for AFM Tip Functionalization. https://wwwjku.at/institut-fuer-biophysik/forschung/linked, 2018; Wildling et al. Linking of sensor molecules with amino groups to aminofunctionalized AFM tips. Bioconjug. Chem. 2011, vol. 22, 1239-1248). AFM tips (PFQNM-LC and MSCT probes, Bruker) were immersed in chloroform for 10 min, rinsed with ethanol, dried with a stream of filtered nitrogen, cleaned for 10 min using an ultraviolet radiation and ozone (UV-0) cleaner (Jetlight), and immersed overnight in an ethanolamine solution (3.3 g of ethanolamine hydrochloride in 6.6 mL of DMSO). The cantilevers were washed three times with DMSO, and two times with ethanol and dried with nitrogen. To ensure a low grafting density of the linker on the AFM tip, 1 mg of acetal-PEG₂₇-NHS was diluted in 0.5 mL of chloroform with 30 μL of trimethylamine (Gruber, supra; Wildling et al., supra). Ethanolamine-coated cantilevers were immersed for 2 h in this solution, washed three times with chloroform, and dried with nitrogen. Cantilevers were then immersed for 10 min in 1% citric acid in milliQ water, washed three times with milliQ water, and dried with nitrogen. Virus solution (80 μL at ˜10⁸ to 10⁹ particles mL⁻¹) was pipetted onto the tips placed on Parafilm (Bemis NA) in a small plastic dish stored within an icebox. A freshly prepared solution of NaCNBH₃ (2 μL at ˜6% wt. in 0.1 M NaOH_{(aq)}) was gently mixed into the virus solution, and the cantilever chips were gently positioned with the cantilevers extending into the virus drop. The icebox was incubated at 4° C. for 1 h. Then, 5 μL of 1 M ethanolamine solution (pH 8) was gently mixed into the drop to quench the reaction. The icebox was incubated at 4° C. for an additional 10 min, and the cantilever chips were removed, washed three times in ice-cold PBS, and stored in individual wells of a multiwell dish containing 2 mL of ice-cold virus buffer (150 mM NaCl, 15 mM MgCl₂, 10 mM Tris, pH adjusted to 7.4) per well until used in AFM experiments. During these functionalization steps, the virus-functionalized cantilevers were never allowed to dry. Transfer of the functionalized AFM cantilevers to virus buffer and then to AFM was rapid (≤20 s) and, during transfer, a drop of virus buffer remained on the cantilever and tip. Cantilevers were used in AFM experiments the same day they were functionalized. Control experiments using confocal imaging showed that in most cases no more than one viral particle was present at the apex of the AFM tip, which interacts with a model surface or cell surface during an AFM experiment.

Preparation of α-SA-Coated Model Surfaces

Biotinylated α2,3-linked sialic acid (SA) was immobilized to plates using the biotin-streptavidin system (Lee et al. Sensing discrete streptavidin-biotin interactions with atomic force microscopy. Langmuir 1994, vol. 10, 354-357) as described (Dupres et al. Nanoscale mapping and functional analysis of individual adhesins on living bacteria. Nat. Methods 2005, vol. 2, 515). Gold-coated silicon substrates were incubated at 4° C. overnight in a 25 μg mL⁻¹ solution of biotinylated bovine serum albumin (BBSA, Sigma-Aldrich) in PBS. After rinsing with PBS, the BBSA surfaces were exposed to a 10 μg mL⁻¹ solution of streptavidin (Sigma-Aldrich) in PBS for 2 h, following by rinsing with PBS. The BBSA-streptavidin surfaces were immersed for 2 h in a 10 μg mL⁻¹ solution of biotinylated 3′-sialyl-N-acetyllactosamine (α2,3-linked SA, Dextra) in PBS, followed by rinsing with PBS. The surfaces showed a homogeneous and stable morphology under repeated scanning and displayed a thickness of ˜2 nm. The thickness of the deposited layer was estimated by scanning a small area (0.5×0.5 μm²) of the surface at high forces to remove the attached biomolecules, followed by imaging larger squares of the same region (2.5×2.5 μm²) at lower force.

Preparation of JAM-A-Coated Model Surfaces

His₆-tagged JAM-A (Bio-Connect Life Science) was immobilized using NTA-His₆ binding chemistry, as described (Dupres et al. 2005, supra; Dufrêne. Life at the nanoscale: atomic force microscopy of live cells. Pan Stanford Publishing, 2011). Gold-coated surfaces were rinsed with ethanol, dried with a gentle nitrogen flow, cleaned for 15 min by UV and ozone treatment, and immersed overnight in ethanol containing 0.05 mM of NTA-terminated (10%) and triethylene glycol(EG)-terminated (90%) alkanethiols. After rinsing with ethanol, the samples coated with alkanethiols were immersed in a 40 mM aqueous solution of NiSO₄ (pH 7.2) for 1 h, rinsed with water, incubated with his₆-tagged JAM-A (0.1 mg mL⁻¹) for 2 h, and rinsed with PBS. The functionalized surfaces were kept hydrated and used immediately after preparation. The surfaces showed a homogeneous and stable morphology under repeated scanning and displayed a thickness of ˜3 nm. The thickness was measured as described for sialic-acid-coated model surfaces.

FD-Based AFM on Model Surfaces

AFM Nanoscope Multimode 8 (Bruker) was used (Nanoscope software v9.1) to conduct FD-based AFM. Virus-functionalized MSCT-D probes (with spring constants calculated using thermal tune, ranging from 0.024 to 0.043 N m⁻¹) (Butt & Jaschke. Calculation of thermal noise in atomic-force microscopy. Nanotechnol. 1995, vol. 6, 1-7) were used to record force curves from 5×5 μm arrays in the force-volume (contact) mode. The approach velocity was kept constant at 1 μm s⁻¹, and retraction velocities were varied from 0.1, 0.2, 1, 5, 10 to 20 μm s⁻¹ to ensure that the energy landscape between the virus and its cognate receptor was probed over a wide range of loading rates. The pulling velocity (v) and loading rate (LR) can be related as follows, LR=ΔF/Δt=k_{eff}·v, where ΔF/Δt being the applied force over time, and k_{eff} the effective spring constant of the system. The ramp size was set to 500 nm and the maximum force to 500 pN, with no surface delay. The sample was scanned using a line frequency of 1 Hz, and 32 pixels were scanned per line (32 lines in total with 1024 data points [FD curves] per retraction speed). All FD-based AFM measurements were obtained in virus buffer at ˜25° C. Force curves were analyzed using the Nanoscope analysis software v1.7 (Bruker). To identify peaks corresponding to adhesion events occurring between particles linked to the PEG spacer and the receptor model surface, the retraction curve before bond rupture was fitted with the worm-like chain model for polymer extension (Bustamante et al. Entropic elasticity of lambda-phage DNA. Science 1994, vol. 265, 1599-1600). The latter expresses the force-extension (F-x) relationship for semi-flexible polymers and is described by the following equation, with lₚ the persistence length, L_{c} the contour length, and k_{b}T the thermal energy:

$$F = \frac{{k}_{b} T}{{l}_{p}} ( \frac{1}{4 {( 1 - \frac{x}{{L}_{c}} )}^{2}} + \frac{x}{{L}_{c}} - 0.25 )$$

Origin software (OriginLab) was used to display the results in dynamic force spectroscopy (DFS) plots to fit histograms of rupture force distributions for distinct loading rate ranges and to apply various force spectroscopy models as described. (Alsteens et al. Nanomechanical mapping of first binding steps of a virus to animal cells. Nat Nanotechnol 2017, vol. 12, 177-183; Newton et al. Combining confocal and atomic force microscopy to quantify single-virus binding to mammalian cell surfaces. Nat. Protoc. 2017, vol. 12, 2275; Delguste et al. in Nanoscale Imaging, 483-514, Springer, 2018 (‘Delguste et al. 2018a’); Delguste et al. Multivalent binding of herpesvirus to living cells is tightly regulated during infection. Science advances 2018, vol. 4, eaat1273 (‘Delguste et al. 2018b’)).

FD-Based AFM and Fluorescence Microscopy on Living Cells

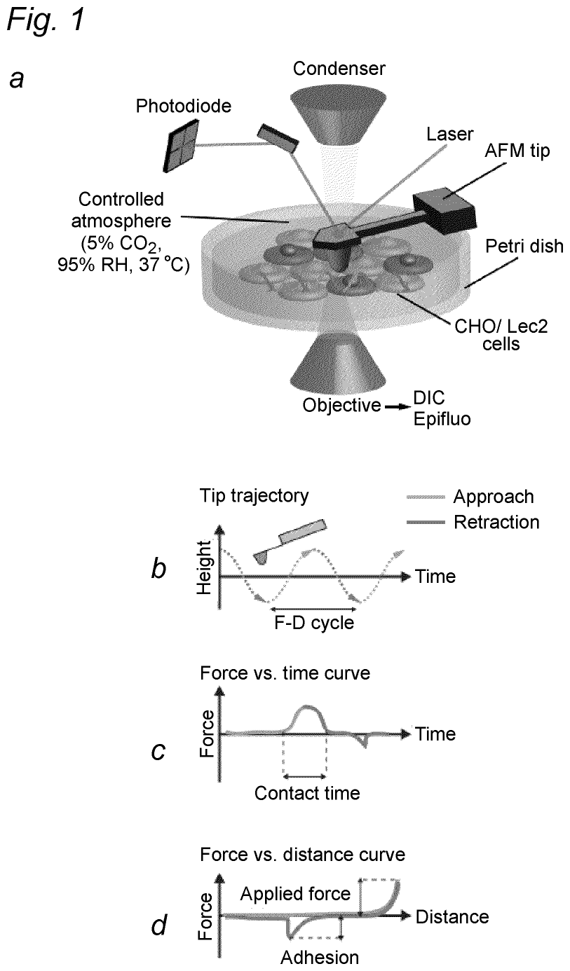

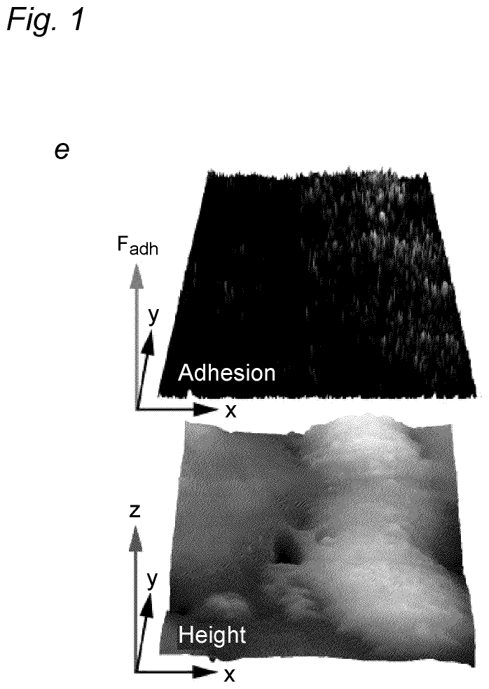

Correlative images were acquired on an AFM (Bioscope Catalyst and Bioscope Resolve, Bruker) operated in the PeakForce QNM mode (Nanoscope software v9.2) to conduct FD-based AFM and coupled to an inverted epifluorescence microscope (Zeiss Observer Z.1) as described (Newton et al. 2017, supra; Knoops et al. Specific Interactions Measured by AFM on Living Cells between Peroxiredoxin-5 and TLR4: Relevance for Mechanisms of Innate Immunity. Cell chemical biology 2018, vol. 25, 550-559, e553). A 40× oil objective (NA=0.95) was used. The AFM was equipped with a 150 μm piezoelectric scanner and a cell culture chamber allowing to control the temperature, the humidity and the CO₂ concentration as described (Alsteens et al. 2017, supra). Overview images of cell surfaces (20-30 μm²) were recorded at imaging forces of ˜500 pN using PFQNM-LC probes (Bruker) having tip lengths of 17 μm, tip radii of 65 nm, and opening angles of 15°. All fluorescence microscopy and FD-based AFM imaging experiments were conducted under cell culture conditions using the combined AFM and fluorescence microscopy chamber (FIG. 1a) at 37° C. in either Mem a, nucleosides or Ham's F12 culture medium, depending on the cell type. A gas mixture of synthetic air with 5% CO₂ at 95% relative humidity using a gas humidifier membrane (PermSelect silicone) was infused at 0.1 L min⁻¹ into the microscopy chamber. The humidity was controlled using a humidity sensor (Sensirion). Cantilevers were first calibrated using the thermal noise method (Huffer & Bechhoefer. Calibration of atomic-force microscope tips. Review of Scientific Instruments 1993, vol. 64, 1868-1873), yielding values ranging from 0.095 to 0.135 N m⁻¹ for PFQNM-LC probes. The AFM tip was oscillated in a sinusoidal fashion at 0.25 kHz with a 750 nm amplitude in the PeakForce Tapping mode. The sample was scanned using a frequency of 0.125 Hz and 256 pixels per line (256 lines). AFM images and FD curves were analyzed using the Nanoscope analysis software (v1.7, Bruker), Origin, and ImageJ (v1.52e). Individual FD curves detecting unbinding events between the virus and cell surface were analyzed using the Nanoscope analysis and Origin software. The baseline of the retraction curve was corrected using a linear fit on the last 30% of the retraction curve. Using the force-time curve, the loading rate (slope) of each rupture event was determined (FIG. 1c). Optical images were analyzed using Zen Blue software (Zeiss) (Alsteens et al. 2017, Newton et al. 2017, Delguste et al. 2018a, Delguste et al. 2018b, all supra).

Monitoring the Effect of SA Addition

The live cell experiments were conducted in the same manner as described above by scanning a suitable field of cells, followed by adding 1 mM of the respective glycan to the culture medium. The same area was scanned again to monitor potential changes after glycan addition. To assess specificity, blocking agents (1 mM Neu5Ac or 10 μg/ml JAM-A Ab [Sigma, #SAB4200468]) were added subsequently.

Monitoring Reovirus Binding to Neuraminidase-Treated Cells

To remove residual cell-surface SA from Lec2 cells, the live cell experiments were conducted in the same manner as described above by scanning a suitable field of cells, followed by treatment with neuraminidase on the microscope stage to allow a second scan of the same field following treatment. The culture medium was removed, and cells were washed with 2 mL PBS (Sigma-Aldrich), treated with Arthrobacter ureafaciens neuraminidase (Sigma-Aldrich) at a final concentration of 40 mUnit/mL in PBS for 1 h, and washed with 2 mL PBS. Experiments were conducted using cell culture medium without any supplements to suppress SA recovery. In addition, 1 mM Neu5Ac was added during a third scan and 10 μg/ml JAM-A Ab during a fourth scan to monitor SA-mediated changes and to assess the specificity of observed interactions, respectively.

Quantification of Reovirus and Lectin Binding

CHO and Lec2 cells (Puro and JAM-A cell lines) were detached from cell-culture dishes using Cellstripper (Cellgro) at 37° C. for 15 min, quenched with the corresponding cell-culture medium, and washed once with PBS. To quantify reovirus binding, cells were adsorbed with 10⁵ fluoresceinated reovirus virions or ISVPs per cell at 4° C. for 1 h. To determine the effect of free SA on reovirus binding, cells were incubated with 1 μM Neu5Ac during virus adsorption. To compare cell-surface SA expression between cell lines, detached cells were adsorbed with fluorescein-labeled wheat germ agglutinin (WGA) at a concentration of 1 μg/mL in PBS containing 5% BSA at 4° C. for 1 h. After respective treatments, cells were washed twice with FACS buffer (PBS containing 2% FBS), stained with LIVE/DEAD Fixable Violet Dead Cell Stain kit (Invitrogen) for 15 min, washed twice again with FACS buffer, and fixed in PBS containing 1% paraformaldehyde. Cells were analyzed using LSRII flow cytometer (BD Bioscience), and reovirus or lectin bound to living cells was quantified using FlowJo software.

Kinetic Analysis of JAM-A-Reovirus Interactions Using BLI

Virus binding to JAM-A was measured on a BLItz® (Pall ForteBio) biolayer interferometer equipped with a Ni2+-NTA biosensor (Pall ForteBio). After loading the chip in a 10 mM NiCl₂ solution for 2 min and running an initial baseline step in milliQ water (1 min), JAM-A (0.2 mg mL⁻¹) was immobilized on the exposed Ni²⁺ ions via its C-terminal His₆ tag for 5 min until the binding signal reached a plateau (complete saturation of the biosensor). Binding of viral particles (T3SA+, T3SA− or ISVP; at 16 nM) in the absence or presence of 1 mM Neu5Ac was measured during a 10 min association step after another baseline step (virus buffer for 1 min). Dissociation was monitored directly after the association step for 10 min during which the virus solution was exchanged with virus buffer. Chip can be regenerated several times by exposing the biosensor to 10 mM Glycine pH 1.7 followed by a neutralization buffer (Kinetics Buffer). The resulting sensorgram (binding over time) was processed and fitted with a nonlinear regression approach using an association and then dissociation fit provided by GraphPad Prism. Virus concentration and time at which dissociation was initiated were constrained to constant values of 16 nM and 17 min, respectively. From that fit, k_{off} and kₒₙ were extracted and KD was calculated.

Single Particle Tracking Using Dynamic Confocal Microscopy Imaging

A coculture of Lec2-JAM-A mCherry and CHO-JAM-A cells was seeded onto a 47-mm glass-bottomed petri dish (WillCo Wells) 1 or 2 d before the experiment to ensure formation of a confluent monolayer on the day of the experiment. Cells were imaged by laser scanning confocal microscopy using a Zeiss LSM 880 microscope with a 561 nm laser for mCherry and a 488 nm laser for Alexa488 and a 40× oil objective (NA=0.95). All experiments were conducted at room temperature with cells maintained in Ham's F12 culture medium and a gas mixture of synthetic air with 5% CO2 at 95% relative humidity that was infused at 0.1 L min⁻¹ into the microscopy chamber using a gas humidifier membrane (PermSelect silicone). The humidity was controlled using a humidity sensor (Sensirion). To ensure virus binding (and not internalization), the cells were placed on ice for 30 min before starting the experiment. After finding an area in which both cell types were adjacent (guided by fluorescence), Alexa488-labelled T3SA+ or T3SA− viruses (10¹² particles/mL) diluted in either F12 Ham's culture medium or 1 mM Neu5Ac solution (on ice) were added to the living cells. The fluorescent signal from both dyes (mCherry and Alexa488) as well as the signal from the PMT channel was recorded for a ˜30 min interval immediately after virus injection at a frame-rate of one image every 13.32 seconds. During recording, the focus was kept constant on the upper surface of cells. Fluorescence images were exported as 12-bit TIFF files, merged into a movie, and further processed using ImageJ (National Institutes of Health, Bethesda). Trajectories were harvested and analyzed using MTrackJ, an ImageJ plugin to track moving viral particles in the movie and obtain track statistics. The latter were further processed using Origin.

Antibody Staining of T3 Reovirus Particles on Cantilever Tips

Individual AFM cantilevers functionalized with virus were placed into wells of a 24-well plate (Corning) and incubated at room temperature for 1 h in 500 μL blocking buffer (PBS with 3% BSA). An antibody against serotype 3 reovirus σ1 protein (9BG5, 0.15 mg mL⁻¹) (Burstin et al. Evidence for functional domains on the reovirus type 3 hemagglutinin. Virology 1982, vol. 117, 146-155) was diluted 1:200 in blocking buffer. Reovirus antibody was prepared by mixing equal volumes of sera from rabbits immunized and boosted with T3D reovirus (Chappell et al. Mutations in type 3 reovirus that determine binding to sialic acid are contained in the fibrous tail domain of viral attachment protein sigmal. J. Virol. 1997, vol. 71, 1834-1841). The mixed serum was pre-adsorbed on a monolayer of CHO cells to deplete nonspecific antibodies. Each cantilever was incubated in 500 μl of the primary antibody solution at room temperature for 1 h. Cantilevers were washed three times with blocking buffer. A secondary antibody solution was prepared by adding a rat anti-mouse IgG2a antibody conjugated to allophycocyanin (APC) fluorophore (Thermo Fisher, catalog #17-4210-82) at 1:400 dilution in blocking buffer. Cantilevers were incubated in 500 μl of the secondary antibody solution at room temperature for 1 h. Finally, cantilevers were washed three times in PBS and stored at 4° C. in the dark until further use. The cantilevers were imaged using the 488 nm laser line of an inverted confocal microscope (Zeiss LSM 880).

AFM Imaging of Reovirus Virions Adsorbed on HOPG Substrate

An 80 μl droplet of virus solution (˜10⁹ particles mL⁻¹) was deposited on a freshly cleaved HOPG (highly oriented pyrolytic graphite, NT-MDT instruments) substrate and incubated at room temperature for 15 min. AFM imaging was conducted in the PeakForce Tapping mode using AC40 Biolever mini AFM tips (nominal spring constant 0.1 Nm⁻¹, Bruker) in PBS buffer. Depending on the desired resolution and scan size, different imaging parameters were used: tip oscillation frequency ranged between 1 and 2 kHz, maximum peak force was 100 pN, scan rates ranged from 0.5 to 2 kHz, peak force amplitudes were between 50 and 100 nm, and resolution was 256 or 512 pixels per line (256 or 512 lines, respectively).

### Example 2—Outer-Capsid Protein Sigma 1 (σ1) Attaches to α-Linked Sialic Acid (α-SA) Glycans Through Multivalent Bonds

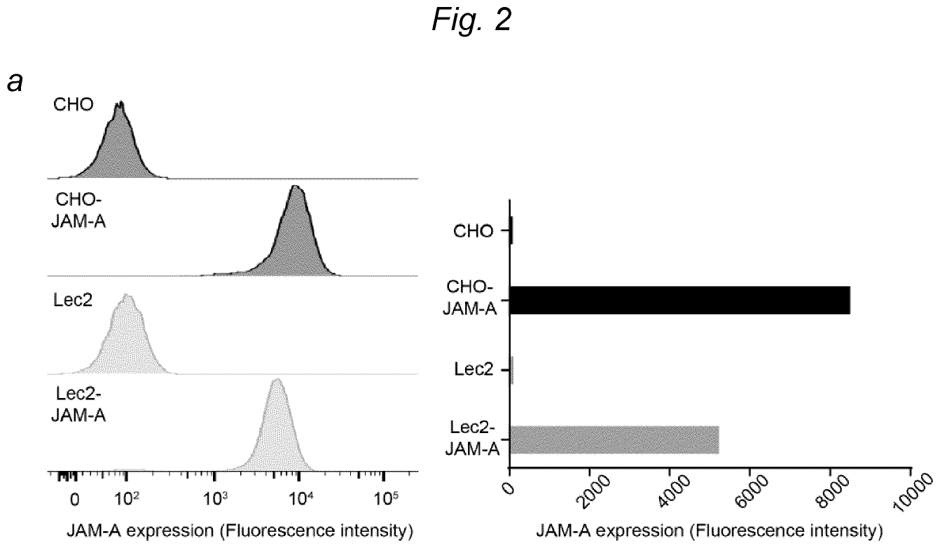

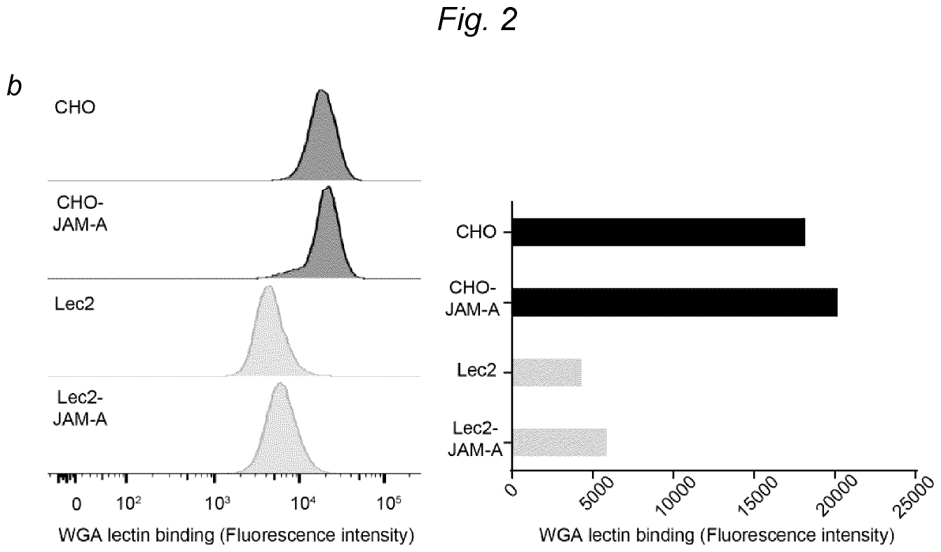

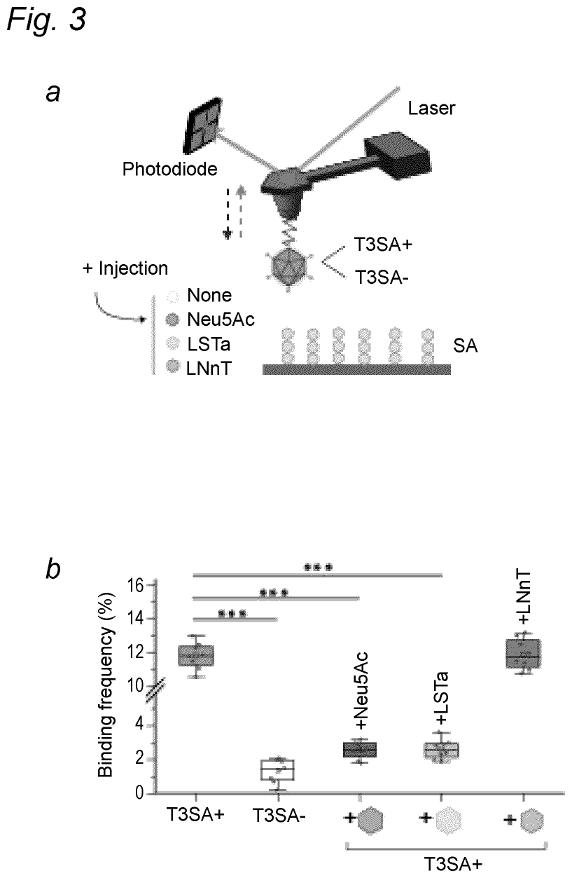

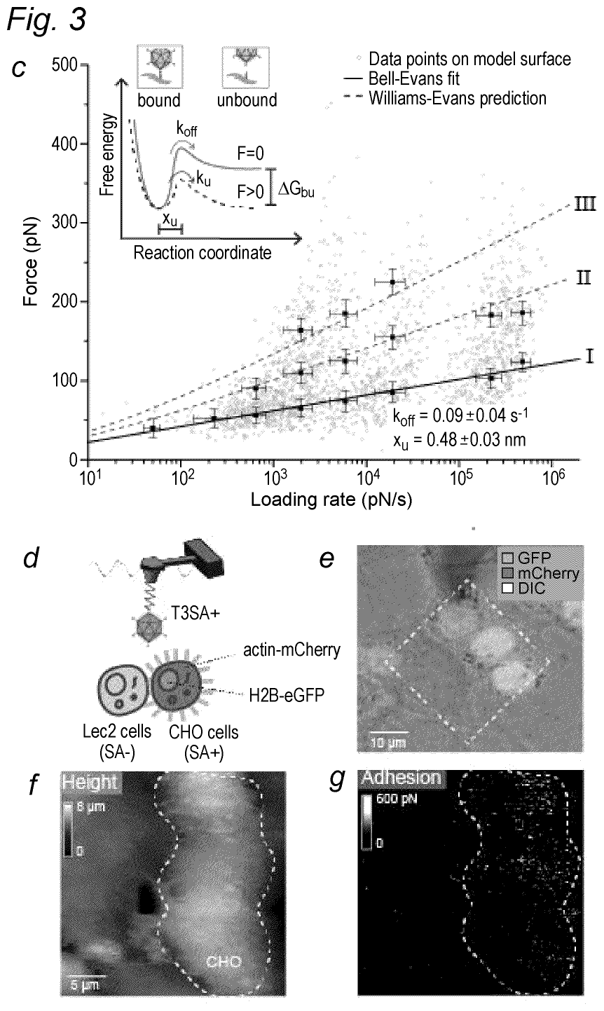

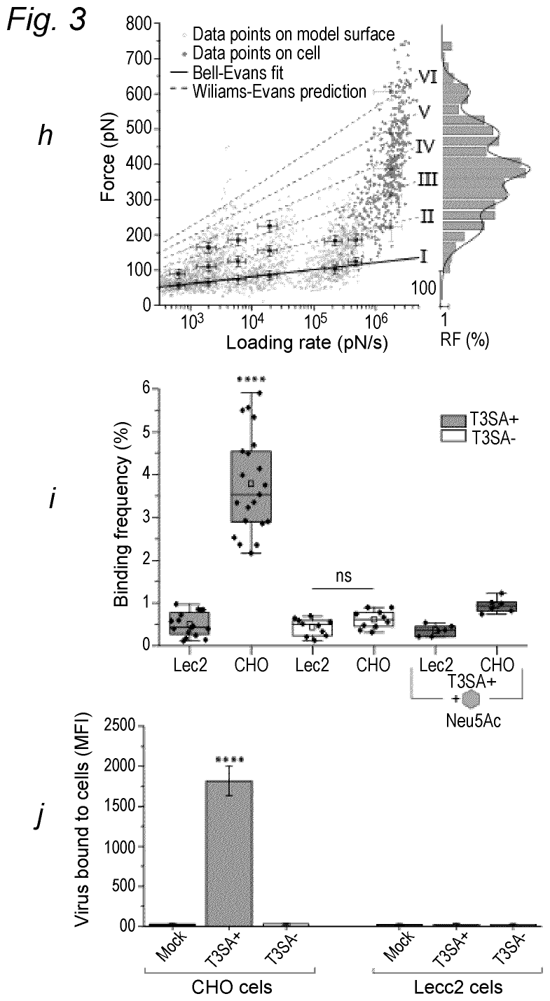

As sigma 1 (σ1) binding to α-linked sialic acid (α-SA) glycans is the first step in reovirus attachment to the cell surface (Barton et al. 2001a, supra), we used atomic force microscopy (AFM) to evaluate the binding strength of reovirus to α-SA using both model surfaces and living cells (FIG. 1 illustrates the principle of force-distance-based AFM; FIG. 12 validates the reovirus virion morphology, tip functionalization, and model surface chemistries; and FIGS. 2 and 13 describe the cell lines used). To mimic cell-surface glycans in vitro, biotinylated-α-SA glycans were immobilized onto streptavidin-coated surfaces to allow virus access to α-SA (Lee et al. Sensing discrete streptavidin-biotin interactions with atomic force microscopy. Langmuir 1994, vol. 10, 354-357, Dupres et al. Nanoscale mapping and functional analysis of individual adhesins on living bacteria. Nat. Methods 2005, vol. 2, 515). Model surfaces were imaged using AFM and validated by scratching the adsorbed layer revealing a deposited layer of ˜1.0±0.3 nm (FIG. 12d). To quantify reovirus binding to α-SA, we covalently attached purified virions of the α-SA-binding reovirus strain T3SA+ (FIG. 12c, showing single virions at the tip apex) to the free end of a long polyethylene glycol (PEG)₂₇ spacer chemically linked to the AFM tip (Alsteens et al. 2017, Newton et al. 2017, Delguste et al. 2018a, all supra). Force-distance curves (FD curves) were recorded to assess the binding strength between T3SA+ virions and α-SA glycans (FIG. 3a-c). Specific adhesion events were observed on 10-15% of retraction FD curves at rupture distances >5 nm, which corresponds to the extension of the PEG linker. To confirm the specificity of these interactions, we conducted additional independent control experiments using (i) an AFM tip attached to a non-SA-binding virus strain, T3SA−, which does not engage α-SA by virtue of a P204L mutation in the α-SA-binding site of σ1 (Reiter et al. Crystal structure of reovirus attachment protein σ1 in complex with sialylated oligosaccharides. PLoS Path. 2011, vol. 7, e1002166); and (ii) competition experiments with soluble α-SA molecules including acetylneuraminic acid (Neu5Ac), sialyl-lacto-N-tetraose (LSTa), or lacto-N-neotetraose (LNnT), a glycan lacking α-SA. As expected, T3SA− did not display significant binding to SA, and the injection of free Neu5Ac and LSTa but not LNnT strongly competed with T3SA+ binding to SA (FIG. 3b). These controls confirm the specificity of interactions and the critical importance of specific residues in σ1 tail region for α-SA binding.

To extract the kinetics of σ1-α-SA interaction, we force-probed the interactions at various force loading rates (Merkel et al. Energy landscapes of receptor-ligand bonds explored with dynamic force spectroscopy. Nature 1999, vol. 397, 50-53) (FIG. 3c and FIG. 1c,d). Using the physiologically relevant direction-of-force application, the σ1-α-SA complex withstood forces in the range of 25 to 400 pN. The force regime is usually associated with the stability of the protein conformation, raising the concern that the reovirus virions linked to the AFM tip could be damaged over time. Because the apices of the cantilevers have radii of ˜40 nm, they only can host a few viral particles, as evidenced by laser-scanning optical microscopy (FIG. 12c). If the reovirus virions at the tip apex were mechanically altered, such alterations would have produced a rapid decrease in the frequency of interactions over time. In contrast, a single cantilever remained active over thousands of interactions and several maps, indicating that tip and surface functionalization sustained the high-forces.

According to the Bell-Evans (BE) model, the σ1-α-SA complex can be described as a simple two-state model, in which the bound state is separated from the unbound state by a single energy barrier located at distance xᵤ=0.48±0.03 nm and crossed with a transition rate k_{off}=0.09±0.04 s⁻¹. We also observed bivalent and trivalent interactions. These multivalent interactions appear as uncorrelated bonds loaded in parallel, as confirmed by the predictive Williams-Evans (WE) model (FIG. 3c, dashed curves II and III). These multivalent interactions are most likely established between σ1 molecules on a single virion attached to the AFM tip and multiple α-SA molecules immobilized on the surface. This hypothesis is supported by the following reasons: (i) σ1 is a trimer with three binding sites; (ii) each virion possess multiple copies (up to 12, corresponding to the virion icosahedral vertices) of the σ1 trimer; (iii) the tip apex bears only one or two virions; and (iv) the unbinding occurs in a single step (a single rupture peak observed in the FD curves). Thus, our in vitro experiments confirm that T3SA+ virions specifically interact with α-SA glycans and that virions rapidly (in the ms range) establish multivalent bonds with α-SA glycans. Buried within the exposed cell-surface glycans, it is conceptually possible that the increasing number of σ1-α-SA complexes provide the virion with the first stable anchorage to the cell surface.

We next confirmed our in vitro results with experiments using living CHO cells that express α-SA (fluorescently labeled with nuclear GFP and actin-mCherry) and Lec2 cells deficient in α-SA expression (˜70-90% deficiency of SA in their glycoproteins and gangliosides) (FIG. 2a, b). Lec2 cells are a mutant clone derived from parental CHO cells that display a substantial reduction in the transport of cytidine-5′-monophosphate-SA into the Golgi (Deutscher et al. Translocation across Golgi vesicle membranes: a CHO glycosylation mutant deficient in CMP-sialic acid transport. Cell 1984, vol. 39, 295-299). Using AFM tips functionalized with T3SA+, we imaged a confluent monolayer of cocultured CHO cells and Lec2 cells using conditions to propagate both cell types (Alsteens et al. 2017, supra) (FIG. 3d-g). Guided by fluorescence, we chose areas in which both cell types were in proximity for use as a direct internal control during AFM imaging (FIG. 3e). AFM height images were recorded together with the corresponding adhesion image, revealing the location of specific adhesion events displayed as bright pixels onto the adhesion map (FIG. 3f, g). Remarkably, CHO cells showed a high density of adhesion events (˜4%, FIG. 3i), whereas Lec2 cells displayed only a sparse distribution of these events (<1%, FIG. 3i), confirming the establishment of specific T3SA+-α-SA bonds on living cells. We also assessed the stability of the virions on the AFM tip apex throughout by recording consecutive maps with the same T3SA+ tip. The presence of virus on the AFM tip during consecutive maps excludes the possibility of virions becoming internalized during our AFM experiments (FIG. 14a-d). In fact, due to the low contact time (in the ˜ms range), the probability of observing internalization events is extremely low. Accordingly, reovirus virions are essentially stationary during the early stage of binding to the cell surface (between 280 and 1500 s). The rupture forces (FIG. 3h, darker grey dots) were in the range of 50-400 pN. Using the WE prediction (established from in vitro data), we deduce that T3SA+ virions establish up to six interactions in parallel with a maximum likelihood of three-to-four interactions (FIG. 3h, dashed lines [II to VI] and histogram). These results suggest that T3SA+ virions, despite a brief contact time of ˜1 ms with the cell surface, are capable of forming multiple interactions in parallel. The σ1 protein forms homotrimers that theoretically could interact simultaneously with up to three α-SA glycans and several σ1 trimers also could interact with the cell surface SA at a given time giving rise to the observed multivalent interactions. These results suggest that the virion uses more than a single σ1 protein for its early attachment to the cell surface. Specificity of the T3SA+-α-SA interaction was validated using three different approaches: (i) probing the same CHO-Lec2 cell mixture first with a T3SA+ tip and then with a T3SA− tip (FIGS. 3i and 14e-h), (ii) blocking specific virus-glycan interactions using 1 mM Neu5Ac (FIGS. 3i and 14i-1) and (iii) flow cytometric analysis of virion binding (FIG. 3j). Observations made using single viral particles were tested at a larger scale by flow cytometry. Cells were incubated with either no virions (Mock) or Alexa Flour 488-labeled T3SA+ or T3SA− virions (10⁵ particles per cell) for 1 h, and the median fluorescence intensity (MFI) of cell-bound virus was determined (FIG. 13). As shown in FIG. 3j, T3SA+ virions mainly bound to CHO cells, whereas almost no binding was detected for Mock or T3SA− virions. Although binding forces appear much larger on CHO cells than on model α-SA surfaces, the specificity of the interaction demonstrated by the controls described above confirms that we are probing the same interactions on both cells and model surfaces. The larger forces are likely the result of a difference in the number of bonds established simultaneously. Together, these results confirm that T3SA+ virions establish multiple, specific interactions with α-SA glycans on living cells.

### Example 3—the σ1 Protein Forms Stable and Multivalent Complexes with JAM-A Receptors

While α-SA engagement can provide the first foothold for reovirus on the cell surface, engaging a specific receptor such as JAM-A facilitates cell entry. To evaluate reovirus binding to JAM-A, we first force-probed T3SA+ or T3SA− virion binding to JAM-A-coated surfaces (FIG. 4a). To mimic physiological conditions, his₆-tagged JAM-A molecules were immobilized in a physiologically oriented manner onto an NTA-Ni²⁺-coated gold surface (Dupres et al. 2005, Dufrêne 2011, all supra) (FIG. 4a), and surface chemistry was validated using AFM scratching experiments (see FIG. 12e). Specific binding forces were observed in the range of 20 to 130 pN and converted into DFS plots for both the JAM-A-T3SA+ (FIG. 4b, upper panel) and JAM-A-T3SA− (FIG. 4b, lower panel) interactions. The JAM-A-reovirus interaction can be defined to have a single energy barrier with xᵤ=0.71±0.05 nm and k_{off}=0.04±0.01 s⁻¹ for the JAM-A-T3SA+ bond and xᵤ=0.48±0.03 nm and k_{off}=0.05±0.03 s⁻¹ for the JAM-A-T3SA− bond. While the off-rates are comparable, the distance to the transition state is smaller for T3SA−, indicating that the energy landscape is described by a narrower energy valley that can accommodate less conformational variability. For T3SA− binding to JAM-A, we frequently observed more larger binding forces, which corresponds to multiple interactions. Together with the narrower energy valley, this observation suggests that the single point mutation in T3SA− σ1 leads to a more rigid and/or compact conformation of the protein. Injection of a JAM-A antibody (AB) reduced the binding frequency, confirming the specificity of virion-JAM-A binding (FIG. 4c).

To define the interaction of reovirus with JAM-A under physiological conditions, we evaluated reovirus binding to JAM-A expressed on living cells. Combined optical and FD-based AFM were conducted using living fluorescently-labeled Lec2 cells (nuclear GFP and actin-mCherry) cocultured with unlabeled Lec2-JAM-A cells (FIG. 4d-g). Mapping of T3SA+-binding using both types of cells highlighted a higher density of adhesion events on Lec2-JAM-A cells (˜3.5%, FIG. 4g,i), with low binding forces (rupture forces) ranging from 50 to 400 pN. Lec2 cells displayed only rare binding events (<0.8%, FIG. 4g,i), confirming specific interactions between cell-surface JAM-A and T3SA+ (see also consecutive mapping in FIG. 15a-d). To eliminate the contribution of the minimal SA expression on Lec2 cells, we also probed the interaction between T3SA− and Lec2-JAM-A cells and observed a similar frequency (˜4.0%, FIG. 4i). In addition, specificity of the interaction was assessed using (i) JAM-A antibody (AB) (FIG. 4i and FIG. 15k-n) and (ii) flow cytometry (FIG. 4j). Similar to results gathered using the in vitro approach, alteration of the SA-binding site does not influence reovirus binding to JAM-A (FIG. 15e-h). Together, these results reveal that T3SA+ establishes stable weak (low multivalency) interactions with JAM-A independent of SA engagement.

To better define the function of JAM-A as a specific receptor on living cells, we analyzed the JAM-A binding forces and overlaid the data onto the DFS plot obtained previously on model surface (FIG. 4h). Compared with the data acquired in vitro, in experiments using living cells we observed establishment of up to four simultaneous uncorrelated virus-receptor bonds (WE-model, dashed curves). Similarly, binding forces measured with T3SA− virions on Lec2-JAM-A cells were extracted and overlaid onto the DFS plot (FIG. 15i, j) providing similar results in terms of binding frequency (FIG. 4i) and number of simultaneous uncorrelated virus-receptor bonds established on living cells (FIG. 15j). These findings suggest that binding to JAM-A is kinetically or sterically less favored than binding to α-SA on cells, for which up to six bonds were observed under similar experimental conditions. However, for both T3SA+ and T3SA−, the majority of adhesion events show rupture forces corresponding to rupture of one or two JAM-A receptor interactions (FIG. 4h, FIG. 15j, histogram), suggesting that binding to JAM-A is kinetically or sterically less favored than binding to α-SA on cells, for which up to six bonds were observed under similar experimental conditions.

### Example 4—Binding to α-Sialylated Glycans Triggers Reovirus Binding to JAM-A

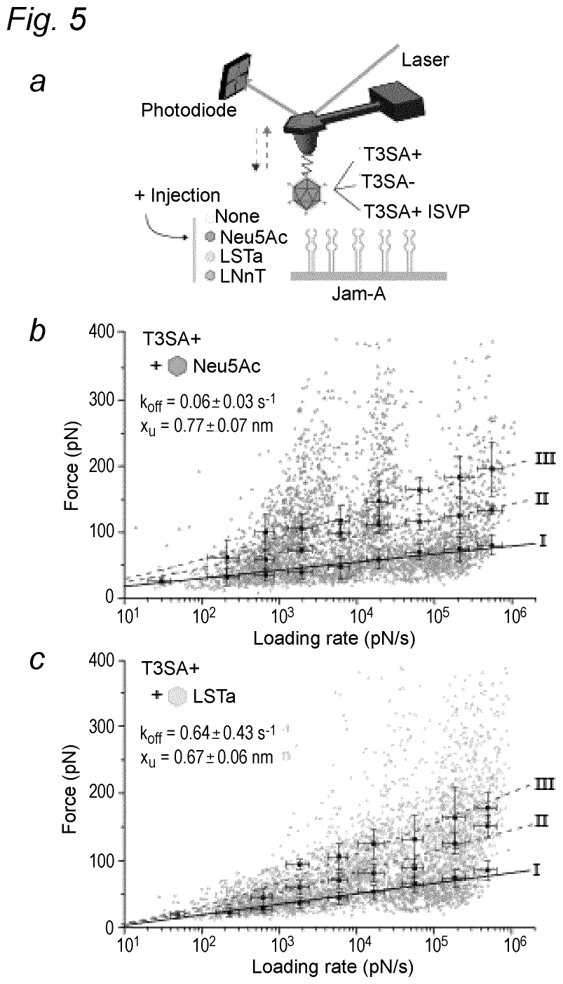

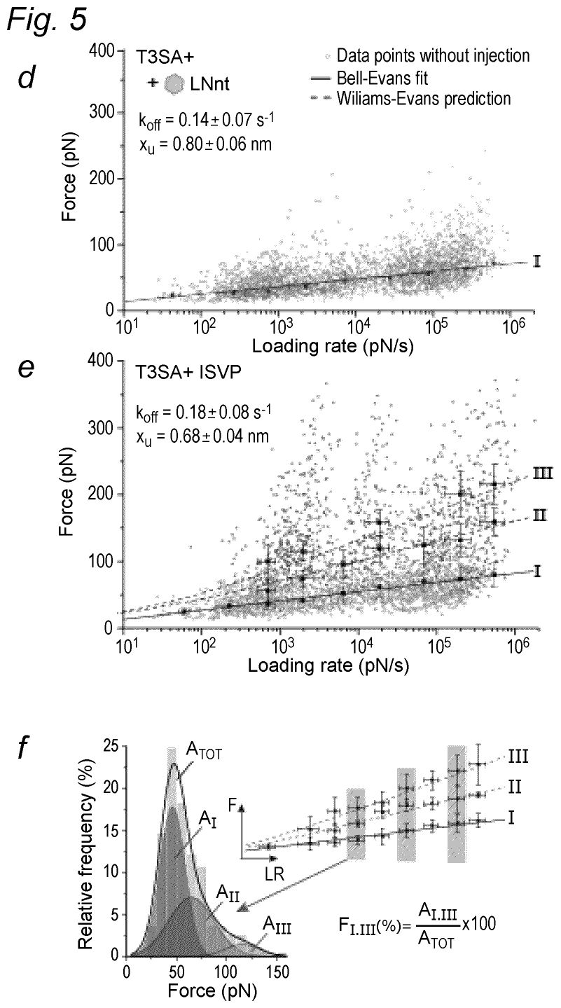

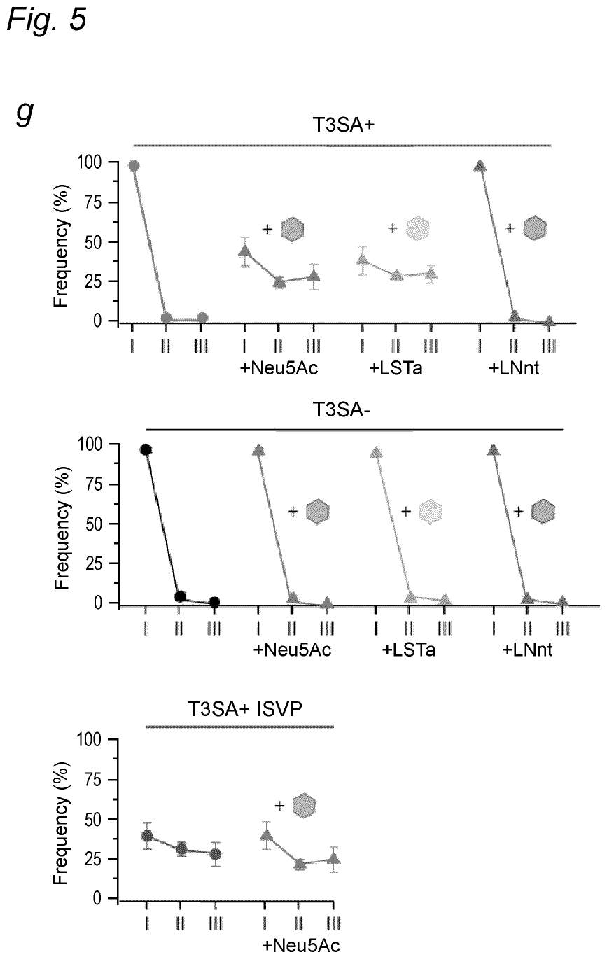

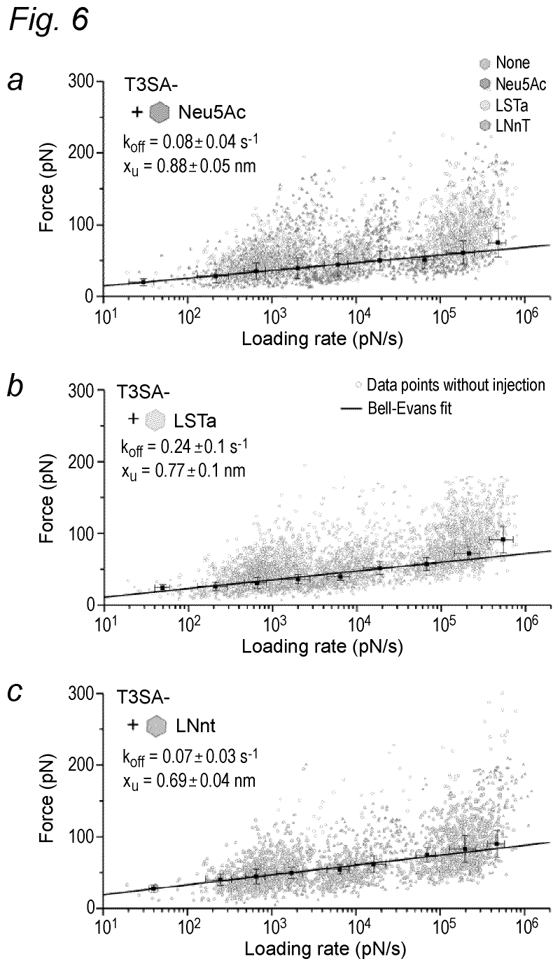

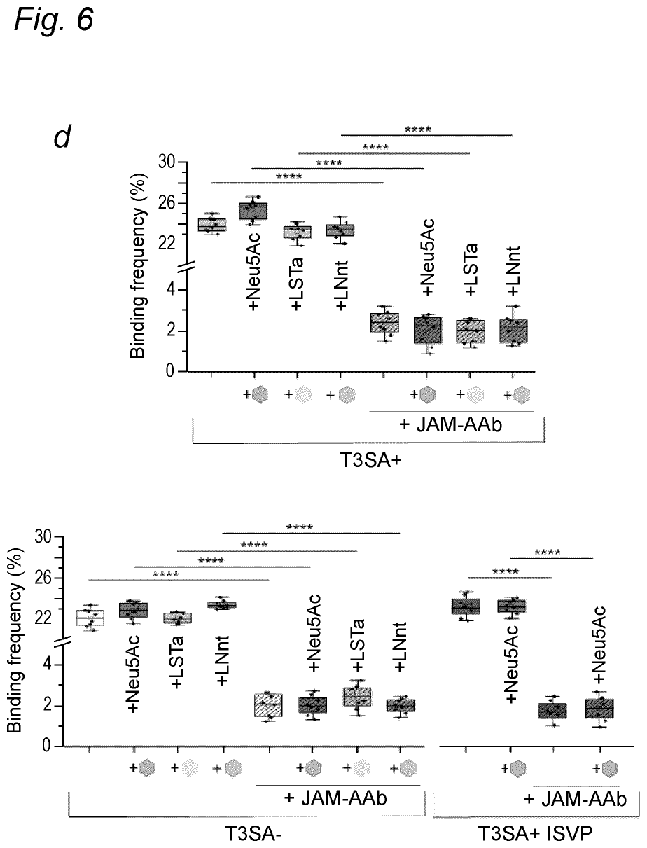

As both α-SA and JAM-A act in concert during reovirus attachment to the cell surface, we assessed reovirus binding to JAM-A in the presence of α-SA first using model surfaces (FIG. 5 and FIG. 6) and then using cells (FIG. 8). While probing T3SA+ binding to JAM-A in vitro, we injected 1 mM glycans with (Neu5Ac and LSTa) and without (LNnT) terminal α-SA. Remarkably, addition of both Neu5Ac and LSTa (FIG. 5b,c) led to a strong effect on the overall binding force (up to 400 pN, compared to 130 pN in absence of α-SA, FIG. 4b), indicating a shift towards multivalent interactions. In the presence of α-SA, three or more simultaneous uncorrelated virus-receptor bonds were observed between T3SA+ and JAM-A (FIG. 5b), giving rise to an increase in the overall avidity. In contrast, incubation with LNnT had no effect on the overall binding of T3SA+ to JAM-A (FIG. 5d), evidencing that the sialyl group is required for the observed behavior. As this behavior is not observed for T3SA− (FIG. 6a-c), the triggering of multivalent interactions by α-SA can be attributed to the formation of a complex between the sialylated glycan and the glycan-binding site in the serotype 3 σ1 tail domain (FIG. 7b).

We hypothesized that the binding of α-SA to σ1 could induce a conformational change in σ1. To investigate this hypothesis, we defined the binding potential of T3SA+ infectious subvirion particles (ISVPs) (FIG. 5e). After proteolytic treatment of virions to generate ISVPs, σ1 appears by cryoEM image reconstructions to assume a more extended state, projecting radially away from the particle surface (Dryden et al. Early steps in reovirus infection are associated with dramatic changes in supramolecular structure and protein conformation: analysis of virions and subviral particles by cryoelectron microscopy and image reconstruction. The Journal of cell biology 1993, vol. 122, 1023-1041) (FIG. 7a). This observation provides a tool to test whether ISVP-JAM-A interactions mimic virion-JAM-A interactions in the presence of α-SA and a potential more extended conformer of σ1. Remarkably, we observed strong interactions between T3SA+ISVPs and JAM-A, which were comparable to the interactions of T3SA+ virions with JAM-A following incubation with α-SA, suggesting that α-SA binding to σ1 induces a conformational change in the protein that enhances its affinity for JAM-A. Moreover, analysis of the number of bonds established between reovirus virions and JAM-A revealed that after binding to α-SA, the reovirus-JAM-A binding potential is increased to an extent similar to that of ISVPs (FIG. 5f,g). We also tested whether binding of ISVPs to JAM-A could be increased by treatment with free α-SA (FIG. 5g, FIG. 16, FIG. 6d). However, there was no observable change in ISVP-JAM-A interactions following α-SA treatment. Therefore, after activation of T3SA+ by α-SA, the σ1 protein appears to undergo a conformational change to a more extended form.

To test whether this activation mechanism occurs in a cellular context, we probed reovirus binding to Lec2-JAM-A cells and monitored the adhesion behavior following injection of Neu5Ac, LSTa, or LNnT (FIG. 8, FIG. 9). As Lec2-JAM-A cells lack sialylated glycans and the binding of reovirus to Lec2 cells is rare (<1%, FIG. 9), we hypothesized that most interactions of reovirus to Lec2-JAM-A cells are established via JAM-A receptors. Therefore, we used this cell line to study the effect of injected glycan derivatives on reovirus-JAM-A interactions and compared the overall binding frequency before and after glycan injection. We observed that T3SA+ binding efficiency increases following injection of only those glycans with terminal α-SA (Neu5Ac and LSTa). T3SA+ virions displayed a significant increase in binding of ˜20-25% following injection of α-SA-glycans (Neu5Ac and LSTa) (FIG. 9; from 3.9 to 4.9% for Neu5Ac and from 3.8 to 4.8% for LSTa). In contrast, we did not observe an increase in binding after injection of LNnT, which lacks α-SA (FIG. 9; from 3.8 to 3.9%). To evaluate whether the residual SA on Lec2 cells could influence our results, we treated Lec2 cells with neuraminidase (40 mUnit/mL for 1 h) to cleave residual cell surface α-SA-glycans (FIG. 8). Similar to the results gathered using untreated cells, we observed an increase in T3SA+ binding of ˜20% after injection of Neu5Ac on neuraminidase-treated Lec2 cells. These results suggest that interaction with SA enhances reovirus binding to JAM-A on the cell surface, and residual SA on the Lec2 cell surface minimally contributes to interaction with reovirus.

To quantify the multivalence of reovirus binding to cell-surface receptors, we analyzed the binding force distribution (FIG. 8p-s). Examination of the adhesion images showing binding events within the high-force range (FIG. 8c,e,h,j,m,o) indicated that the binding frequency of those events significantly increased after incubation with α-SA. This observation is consistent with our in vitro data confirming a change in the virion binding potential triggered by α-SA incubation. Analysis of the number of bonds established between a T3SA+ virion and JAM-A molecules on the cell surface (FIG. 8t) confirmed this enhanced multivalence. Following activation by α-SA, the mean number of bonds increased by a factor of 2.5, from 1.8±0.3 bonds before to 4.5±1.2 bonds after glycan injection. Together, these data evidence that α-SA binding to σ1 enhances the affinity of σ1 for JAM-A, likely (but without being bound to any hypothesis) as a consequence of inducing a conformational change.

### Example 5—Triggering Multivalent Anchorage of Reovirus Alters Virion Binding and Diffusion Potential

To evaluate how α-SA influence the dynamics of reovirus binding to JAM-A, we used optical bio-layer interferometry (BLI) and fluorescence microscopy based single-particle tracking (SPT).

Using BLI, we quantified reovirus binding to Ni²⁺-NTA-biosensors coated with JAM-A and also tested the influence of free Neu5Ac on the overall avidity. The data show that T3SA+ and T3SA− bind to JAM-A with high avidity (KD ˜nM range) (FIG. 10a,b). As expected, free Neu5Ac had no influence on T3SA− binding. In marked contrast, T3SA+ virions incubated with free Neu5Ac and ISVPs have a much higher avidity for JAM-A, reaching a very high affinity (KD ˜pM range) (FIG. 10a). These observations are consistent with our AFM data showing a binding potential of T3SA+ virions incubated with α-SA compounds comparable to that of ISVPs.

Reovirus binding to the surface of living cells also was evaluated dynamically by SPT using high-speed confocal microscopy. Fluorescently labeled T3SA+ virions were incubated with mCherry-labelled Lec2-JAM-A cells cocultured with CHO-JAM-A cells. Time-lapse series of images were recorded in the presence or absence of 1 mM Neu5Ac (FIG. 10c-f). T3SA+ particles diffuse more rapidly on cells lacking SA (Lec2 cells), whereas the particles are more static on cells expressing both SA and JAM-A receptors (CHO-JAM-A). Injection of viral particles together with Neu5Ac leads to a significant decrease of their diffusion potential (FIG. 10f). Analysis of at least 15 particle trajectories on each cell type revealed that virions diffuse over greater distances and with increased speed on cells lacking α-SA glycans (FIG. 10g). Moreover, injection of free SA reduced T3SA+ diffusion on the cell surface, presumably by the capacity to mediate multivalent interactions with JAM-A, and significantly increased the number of particles bound to CHO-JAM-A cells (FIG. 10g). Importantly, this effect was not observed for non-SA-binding strain T3SA− (FIG. 10g). Together, these observations strengthen our conclusion that σ1 binding to α-SA induces a conformational change in σ1 that leads to an increase in the multivalent attachment of the virus to cell-surface receptors.

Conclusions from Examples 2-5

We used AFM in combination with confocal microscopy to force-probe and characterize essential components of the cellular plasma membrane facilitating reovirus entry into cells. The crystal structure of reovirus attachment protein σ1 reveals an elongated fiber with a tail domain formed by α-helical coiled coil and triple-β spiral and a head domain formed by a compact eight-stranded β-barrel. As the principal factor initiating reovirus entry, σ1 of serotype3 reovirus contains receptor-binding regions in both tail and head domains. While the triple-β spiral in the tail domain binds to α-SA, the head domain binds to JAM-A.

Using single-virus force spectroscopy, we investigated reovirus binding to both α-SA and JAM-A by quantifying the binding strength to each receptor, and extracting kinetic parameters of single bonds. AFM experiments conducted using model surfaces and living cells enabled us to determine the multivalency of the interaction in a cellular context. During reovirus binding to the cell surface, we observed that three parallel interactions with α-SA and two to three interactions with JAM-A are favored. This finding suggests that receptor-binding domains on each monomer of the σ1 trimer can independently engage receptors α-SA and JAM-A. These results also establish that the number of established bonds contributes to the overall avidity of virus-receptor binding. Without wishing to be bound by any theory or hypothesis, the affinity for an individual receptor molecule might be very low (in the mM range for single protein-glycan interactions) but can increase to remarkable avidity values (in the nM range) as a consequence of multivalent interactions. In the case of reovirus, after landing on the cell surface and binding to receptors, the virus adheres to a confined location from which it will be endocytosed in a signal-induced manner. Maximizing the number of bonds may help reduce the lateral diffusion of virions. In this context, extraction of the number of virus-receptor bonds at a single-virion level, as conducted herein, allows us to understand the initiation of the infection process.

Attachment factors often mediate weak interactions that lack specificity and serve to tether the virus to the cell surface allowing access to specific entry mediators. We unexpectedly discovered that the binding of the reovirus σ1 tail to α-SA enhances the binding of the σ1 head to JAM-A. Since the α-SA-mediated increased affinity of virions for JAM-A mimics the JAM-A affinity of ISVPs, which contain a more extended conformer of σ1, without wishing to be bound by any hypothesis, our data corroborates that σ1 binding to α-SA triggers a conformational change in the σ1 protein that renders the JAM-A-binding site more accessible. From a molecular point of view, an attractive hypothesis is that α-SA binding to the σ1 tail induces a cis to trans isomerization of the L203-P204 bond resulting in an important conformational change towards a more extended form of the protein (FIG. 10h). Binding to α-SA, which is engaged with lower affinity, serves as the initial attachment event and triggers a conformational change in the σ1 protein that enhances further specific interactions with the high-affinity JAM-A receptor. Our findings provide unique opportunities to manipulate reovirus binding efficiency and infectivity for vaccine and oncolytic applications.

### Example 6—Infection of In Vitro Cultured Cells by Reovirus

T3SA+ and T3SA− reovirus stocks are prepared and purified virions obtained as set forth in Example 1. Lec2, Lec2-JAM-A, CHO and CHO-JAM-A cells are prepared and cultured as set forth in Example 1.

The cells are infected with the reovirus at a suitable multiplicity of infection (MOI) in batch suspensions or in monolayers and infectivity is analysed by an appropriate assay, such as quantification of viral protein expression in infected cells or a plaque assay.

T3SA+ virus infectivity increases as follows Lec2<CHO<<Lec2-JAM-A<CHO-JAM-A.

T3SA− virus infectivity increases as follows Lec2 CHO<<Lec2-JAM-A˜CHO-JAM-A.

Anti-JAM-A antibody neutralizes T3SA+ or T3SA− virus infectivity.

Further experiments are done with Lec2-JAM-A and CHO-JAM-A cells.

Sialylated glycans Neu5Ac or LSTa are co-administered with the virus (admixed with the virus, or added to the cells from a separate vial) and significantly increase infectivity of T3SA+ towards Lec2-JAM-A and CHO-JAM-A, compared to T3SA+ without Neu5Ac or LSTa.

Sialylated glycans Neu5Ac or LSTa do not affect infectivity of T3SA− towards Lec2-JAM-A or CHO-JAM-A.

Non-sialylated glycan LNnT does not affect infectivity of T3SA+ towards Lec2-JAM-A and CHO-JAM-A, compared to T3SA+ without LNnT.

### Example 7—Infection of Mice by Reovirus

T3SA+ and T3SA− reovirus stocks are prepared and purified virions obtained as set forth in Example 1.

Mice are inoculated perorally with 10⁷ or 10¹⁰ plaque forming units (PFU) of the virus and real time PCR is used to quantify viral titres in the small intestine 4-7 days post infection.

Illustrative ways of detecting the virus infection include PCR, fluorescence imaging or ELISA (see for example Bouziat et al. Reovirus infection triggers inflammatory responses to dietary antigens and development of celiac disease. Science 2017, vol. 356, pp. 44-50; Montufar-Solis and Klein. Experimental Intestinal Reovirus Infection of Mice: What We Know, What We Need to Know, Immunol Res. 2005, vol. 33, 257-265) or bioluminescence assay (see for example Pan et al. Visualizing influenza virus infection in living mice. Nature Communications 2013, vol. 4, 2369).

Alternatively, the respiratory tract of mice is infected and infectivity determined essentially as described in Flano et al. (Methods used to study respiratory virus infection. Curr Protoc Cell Biol. 2009, CHAPTER: Unit-26.3). Standard procedures are used including (i) basic techniques for mouse infection, tissue sampling and preservation, (ii) determination of viral titers, isolation and analysis of lymphocytes and dendritic cells using flow-cytometry, and (iii) lung histology, immunohistochemistry and in situ hybridization.

In these models, T3SA+ is more infective compared to T3SA−. Sialylated glycans Neu5Ac or LSTa are co-administered with the virus (admixed with the virus, or added to the mice from a separate vial) and significantly increase infectivity of T3SA+ compared to T3SA+ without Neu5Ac or LSTa. Sialylated glycans Neu5Ac or LSTa do not affect infectivity of T3SA−. Non-sialylated glycan LNnT does not affect infectivity of T3SA+.

### Example 8—Vaccines

The following compositions illustrate vaccines embodying the principles of the present invention.

Avian reovirus vaccine A. A vaccine composition comprises living attenuated reovirus, strain S 1133 (available as Nobilis® REO 1133 from MSD Animal Health), at least 10³ CCID₅₀ per dose of 0.2 ml, supplied with 1 mM N-acetylneuraminic acid (Neu5Ac).

Avian reovirus vaccine B. A vaccine composition comprises living attenuated reovirus, strain S 1133, at least 10³ CCID₅₀ per dose of 0.2 ml, supplied with 10 mM N-acetylneuraminic acid.

Avian reovirus vaccine C. A vaccine composition comprises living attenuated reovirus, strain S 1133, at least 10³ CCID₅₀ per dose of 0.2 ml, supplied with 1 mM sialyl-lacto-N-tetraose a (LSTa).

Avian reovirus vaccine D. A vaccine composition comprises living attenuated reovirus, strain S 1133, at least 10³ CCID₅₀ per dose of 0.2 ml, supplied with 10 mM LSTa.

Human reovirus vaccine A. Live attenuated human reovirus Type 1 strain Lang, at least 10⁶ CCID₅₀ per oral dose of 1.5 ml, supplied with 1 mM Neu5Ac, or 1 mM LSTa, or 10 mM Neu5Ac, or 10 mM LSTa. The suspension contains sucrose as stabiliser.

Human reovirus vaccine B. Live attenuated human reovirus Type 2 strain Jones, at least 10⁶ CCID₅₀ per oral dose of 1.5 ml, supplied with 1 mM Neu5Ac, or 1 mM LSTa, or 10 mM Neu5Ac, or 10 mM LSTa. The suspension contains sucrose as stabiliser.

Human rotavirus vaccine A. Live attenuated human rotavirus strain RIX4414 (available as Rotarix® from GlaxoSmithKline), at least 10⁶ CCID₅₀ per oral dose of 1.5 ml, supplied with 1 mM Neu5Ac, or 1 mM LSTa, or 10 mM Neu5Ac, or 10 mM LSTa. The suspension contains sucrose as stabiliser.

Human rotavirus vaccine A. Live attenuated human-bovine rotavirus reassortants type G1 (not less than 2.2×10⁶ infectious units), type G2 (not less than 2.8×10⁶ IU), type G3 (not less than 2.2×10⁶ IU), type G4 (not less than 2.0×10⁶ IU), and type P1A[8] (not less than 2.3×10⁶ IU) (available as part of RotaTeq® from Merck Vaccines), per oral dose of 2.0 ml, supplied with 1 mM Neu5Ac, or 1 mM LSTa, or 10 mM Neu5Ac, or 10 mM LSTa. The suspension contains sucrose as stabiliser.

### Example 9—Oncolytic Preparations

The following compositions illustrate oncolytic preparations embodying the principles of the present invention.

Oncolytic preparation A. A non-pathogenic, oncolytic human wild-type reovirus type 3 strain Dearing, 3×10¹⁰ CCID₅₀ per dose, configured for intravenous administration, supplied with 1 mM Neu5Ac, or 1 mM LSTa, or 10 mM Neu5Ac, or 10 mM LSTa.

Oncolytic preparation B. A non-pathogenic, oncolytic human wild-type reovirus type 3 strain Dearing, covalently coupled to a single domain VHH antibody against a tumor specific antigen. The antibody contains Neu5Ac or LSTa covalently coupled thereto. 3×10¹⁰ CCID₅₀ per dose, configured for intravenous administration.

1. A composition or a kit-of-parts comprising: i) a virus which is a member of the Reoviridae family and ii) sialic acid, a molecule comprising at least one sialic acid moiety, or a combination thereof.

2. The composition or kit-of-parts according to claim 1, wherein the Reoviridae virus displays host tropism for at least one vertebrate species.

3. The composition or kit-of-parts according to claim 1, wherein the Reoviridae virus displays host tropism for at least one mammalian species.

4. The composition or kit-of-parts according to claim 1, wherein the Reoviridae virus displays host tropism for humans.

5. The composition or kit-of-parts according to claim 1, wherein the Reoviridae virus is an Orthoreovirus, Orbivirus, or Rotavirus.

6. The composition or kit-of-parts according to claim 1, wherein the Reoviridae virus comprises an outer capsid and an inner core.

7. The composition or kit-of-parts according to claim 1, wherein the Reoviridae virus comprises an outer capsid protein capable of binding to a host cell surface receptor, wherein the sialic acid or the molecule comprising the at least one sialic acid moiety causes said outer capsid protein to adopt a more elongated or extended conformation on the Reoviridae virus compared to the conformation in the absence of the sialic acid or the molecule comprising the at least one sialic acid moiety.

8. The composition or kit-of-parts according to claim 7, wherein the outer capsid protein is sigma-1 protein.

9. The composition or kit-of-parts according to claim 1, wherein the sialic acid is N-substituted neuraminic acid, or wherein the at least one sialic acid moiety is an N-substituted neuraminic acid moiety.

10. The composition or kit-of-parts according to claim 1, wherein the sialic acid is N-acetylneuraminic acid (Neu5Ac) or N-glycolylneuraminic acid (Neu5Gc); or wherein the at least one sialic acid moiety is a Neu5Ac or Neu5Gc moiety.

11. The composition or kit-of-parts according to claim 1, wherein the sialic acid is Neu5Ac or wherein the at least one sialic acid moiety is a Neu5Ac moiety.

12. The composition or kit-of-parts according to claim 1, wherein the molecule comprises or consists of an oligosaccharide or a polysaccharide comprising the at least one sialic acid moiety as a terminal moiety.

13. The composition or kit-of-parts according to claim 1, wherein the Reoviridae virus is an oncolytic virus.

14. The composition or kit-of-parts according to claim 13, wherein the oncolytic Reoviridae virus is linked to a binding agent capable of specifically binding to neoplastic cells.

15. The composition or kit-of-parts according to claim 1, wherein the Reoviridae virus is an attenuated live virus.

16. (canceled)

17. A method of treating a neoplastic disease in a subject in need thereof, comprising administering to the subject i) an oncolytic virus which is a member of the Reoviridae family and ii) sialic acid, a molecule comprising at least one sialic acid moiety, or a combination thereof.

18. A method of immunizing a subject against Reoviridae virus, comprising administering to the subject i) attenuated live Reoviridae virus and ii) sialic acid, a molecule comprising at least one sialic acid moiety, or a combination thereof.

19. The composition or kit-of-parts according to claim 9, wherein said N-substituted neuraminic acid or said N-substituted neuraminic acid moiety is further O-substituted.

20. The composition or kit-of-parts according to claim 10, wherein one or more hydroxyl groups of said Neu5Ac or Neu5Gc, or wherein one or more hydroxyl groups of said Neu5Ac or Neu5Gc moiety, are each independently substituted.

21. The composition or kit-of-parts according to claim 20, wherein one or more hydroxyl groups of said Neu5Ac or Neu5Gc, or wherein one or more hydroxyl groups of said Neu5Ac or Neu5Gc moiety, are each independently substituted with acetyl, methyl, lactyl, sulphate or phosphate.

22. The composition or kit-of-parts according to claim 11, wherein the composition or kit-of-parts comprises Neu5Ac.

23. The composition or kit-of-parts according to claim 14, wherein the binding agent is an antibody.

24. The composition or kit-of-parts according to claim 14, wherein the sialic acid, the molecule comprising the at least one sialic acid moiety, or both, is also linked to the binding agent.


SEQUENCE LISTING (2 sequences)

SEQ ID NO: 1 - protein, 299 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV

SEQ ID NO: 2 - protein, 250 aa, Homo sapiens
MGTKAQVERKLLCLFILAILLCSLALGSVTVHSSEPEVRIPENNPVKLSCAYSGFSSPRVEWKFDQGDTTRLVCYNNKITVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV